SEQUENCE LISTING (44 sequences)

SEQ ID NO: 1 - DNA, 19 bp, Artificial Sequence
  tetO-sequence
tccctatcagtgatagaga

SEQ ID NO: 2 - DNA, 28 bp, Artificial Sequence
  thermosensitive mutant sequence
ctgtgcttctagaattatgtggggggaa

SEQ ID NO: 3 - DNA, 32 bp, Artificial Sequence
  thermosensitive mutant sequence
agctttccccccacataattctagaagcacag

SEQ ID NO: 4 - DNA, 53 bp, Artificial Sequence
  primer
aggtttaaactacgggatccgtgcaccatggataaagttttaaacagagagga

SEQ ID NO: 5 - DNA, 28 bp, Artificial Sequence
  primer
ccgaattctttatgtttcaggttcaggg

SEQ ID NO: 6 - protein, 6 aa, Artificial Sequence
  basis of LTR from shuttle plasmid pLV.MHCK7.eGFP
WHVPRE

SEQ ID NO: 7 - DNA, 22 bp, Artificial Sequence
  primer
ggctatgaggaatggctattga

SEQ ID NO: 8 - DNA, 19 bp, Artificial Sequence
  primer
aagtagggctcgaacttcc

SEQ ID NO: 9 - DNA, 20 bp, Artificial Sequence
  primer
tgacccacgagctgttaatc

SEQ ID NO: 10 - DNA, 20 bp, Artificial Sequence
  primer
ggtgttctctcctgttctgt

SEQ ID NO: 11 - DNA, 21 bp, Artificial Sequence
  primer
caaggtggtacacgaagctca

SEQ ID NO: 12 - DNA, 20 bp, Artificial Sequence
  primer
acagtgctgcccctggagta

SEQ ID NO: 13 - DNA, 18 bp, Artificial Sequence
  primer
ggctctctgggaaactgg

SEQ ID NO: 14 - DNA, 18 bp, Artificial Sequence
  primer
gaggtgcctagtccttgc

SEQ ID NO: 15 - DNA, 23 bp, Artificial Sequence
  primer
caactttagcaggatctaccatc

SEQ ID NO: 16 - DNA, 21 bp, Artificial Sequence
  primer
agagcttcattgaggagtcca

SEQ ID NO: 17 - DNA, 18 bp, Artificial Sequence
  primer
tagcctcctcaacatccc

SEQ ID NO: 18 - DNA, 18 bp, Artificial Sequence
  primer
ccatgtctgcacttagcc

SEQ ID NO: 19 - DNA, 23 bp, Artificial Sequence
  primer
ggatcttacatgcttctgtaacc

SEQ ID NO: 20 - DNA, 20 bp, Artificial Sequence
  primer
cagagaacttgtcctgttgc

SEQ ID NO: 21 - DNA, 17 bp, Artificial Sequence
  primer
acagccacatggtctcc

SEQ ID NO: 22 - DNA, 20 bp, Artificial Sequence
  primer
ccagttcaagttggtcaagg

SEQ ID NO: 23 - DNA, 22 bp, Artificial Sequence
  primer
ggttgtggtcatactagaaggg

SEQ ID NO: 24 - DNA, 20 bp, Artificial Sequence
  primer
gcagtcatatctgccttggg

SEQ ID NO: 25 - DNA, 21 bp, Artificial Sequence
  primer
ggaagacccggaggtaataag

SEQ ID NO: 26 - DNA, 21 bp, Artificial Sequence
  primer
ctccactcagctatccttcac

SEQ ID NO: 27 - DNA, 23 bp, Artificial Sequence
  primer
gattcagatcacgaggattatgg

SEQ ID NO: 28 - DNA, 21 bp, Artificial Sequence
  primer
gtgctgttgaagatgatcagg

SEQ ID NO: 29 - DNA, 21 bp, Artificial Sequence
  primer
ctaccagatcctgtccaacaa

SEQ ID NO: 30 - DNA, 19 bp, Artificial Sequence
  primer
gcacatcaaaggcgctatc

SEQ ID NO: 31 - DNA, 21 bp, Artificial Sequence
  primer
caccaagagggtcatccaata

SEQ ID NO: 32 - DNA, 21 bp, Artificial Sequence
  primer
ccaaatcgggaggagttatca

SEQ ID NO: 33 - DNA, 18 bp, Artificial Sequence
  primer
tcaatgttcgagcaagcc

SEQ ID NO: 34 - DNA, 20 bp, Artificial Sequence
  primer
ctgacttgcagatgattccg

SEQ ID NO: 35 - DNA, 20 bp, Artificial Sequence
  primer
ctcccacctttaggagaagg

SEQ ID NO: 36 - DNA, 19 bp, Artificial Sequence
  primer
cgaggcatcaggttaggtc

SEQ ID NO: 37 - DNA, 20 bp, Artificial Sequence
  primer
ccaagggcttcttcctcttc

SEQ ID NO: 38 - DNA, 22 bp, Artificial Sequence
  primer
ctcatcttctaccggcatcttc

SEQ ID NO: 39 - DNA, 20 bp, Artificial Sequence
  primer
gtaacccgttgaaccccatt

SEQ ID NO: 40 - DNA, 20 bp, Artificial Sequence
  primer
ccatccaatcggtagtagcg

SEQ ID NO: 41 - DNA, 20 bp, Artificial Sequence
  primer
aactggcctttgatgttggc

SEQ ID NO: 42 - DNA, 22 bp, Artificial Sequence
  primer
aatatgaggtcgtctccaaccc

SEQ ID NO: 43 - DNA, 23 bp, Artificial Sequence
  primer
ccattgtgccctgacgaacctta

SEQ ID NO: 44 - DNA, 21 bp, Artificial Sequence
  primer
gctgcctccagcttccacaca

(19) United States
(12) Patent Application Publication
(10) Pub. No.: US 2020/0032213 A1
(43) Pub. Date: Jan. 30, 2020

(54) CONDITIONALLY IMMORTALIZED CELLS AND METHODS FOR THEIR PREPARATION

(71) Applicant: Academisch Ziekenhuis Leiden h.o.d.n. LUMC, Leiden (NL)

(72) Inventors: Antonius Andrianus Franciscus de Vries, Leiden (NL); Daniel Antonie Pijnappels, Leiden (NL); Martin Jan Schaly, Leiden (NL)

(21) Appl. No.: 16/480,280

(22) PCT Filed: Jan. 23, 2018
(86) PCT No.: PCT/NL2018/050046

§ 371 Date: Jul. 23, 2019

(30) Foreign Application Priority Data
Jan. 23, 2017 (EP) 17152653.6
Oct. 10, 2017 (EP) 17195811.9

(51) Int. Cl. C12N 5/077

(52) U.S. Cl. CPC C12N 5/0657; C12N 2510/04; C12N 2840/002; C12N 2830/006; C12N 2830/003

(57) ABSTRACT

The present disclosure relates to genetically modified conditionally immortalized cells and cell lines, provided with a vector comprising a promoter sequence, at least one immortalization gene operably linked to the promoter sequence, a gene coding for an inducible regulator operably linked to the promoter sequence and a response element for the inducible regulator. The present disclosure further relates to methods for their preparation, and to immortalization and differentiation of such cells.

### CROSS-REFERENCE TO RELATED APPLICATIONS

This application is a national phase entry under 35 U.S.C. § 371 of International Patent Application PCT/NL2018/050046, filed Jan. 23, 2018, designating the United States of America and published as International Patent Publication WO 2018/135948 A1 on Jul. 26, 2018, which claims the benefit under Article 8 of the Patent Cooperation Treaty to European Patent Application Serial No. 17152653.6, filed Jan. 23, 2017 and European Patent Application Serial No. 17195811.9, filed Oct. 10, 2017.

### STATEMENT ACCORDING TO 37 C.F.R. § 1.821(c) or (e)—SEQUENCE LISTING SUBMITTED AS ASCII TEXT FILE

Pursuant to 37 C.F.R. § 1.821(c) or (e), a file containing an ASCII text version of the Sequence Listing has been submitted concomitant with this application, the contents of which are hereby incorporated by reference.

### TECHNICAL FIELD

The application relates to methods for reversible immortalization of cells, cells and cell lines obtained with such methods and vectors and kits of parts used in such methods.

### BACKGROUND

There is a large interest in using cultures of mammalian and avian cells for mechanistic studies, inter alia to establish in vitro models of disease and to test new treatment modalities, for screening of drugs and toxins and as cell sources for tissue engineering and cell therapy. This especially holds true for parenchymal/fully differentiated cells of human origin. Proliferation and (terminal) differentiation are mutually exclusive processes in most cell lineages. Accordingly, differentiated mammalian cells usually cannot be expanded ex vivo, which severely limits in vitro studies of especially (parenchymal) cells derived from hard-to-obtain (healthy) tissues (e.g., human brain, lung, heart, kidney, liver).

One way to obtain large numbers of cells from small tissue samples is by immortalizing the cells directly after their isolation from tissue samples followed by their ex vivo expansion in a dedifferentiated state and their redifferentiation using specific medium formulations. This, however, rarely yields cells in an advanced state of differentiation due to the continued presence of proliferation stimuli. For instance, due to the paucity of human heart tissue and the extremely low mitotic activity of postnatal human cardiomyocytes, cardiac research has strongly relied on experiments in animals or with primary cardiomyocytes of animal origin. Recently, cardiomyocytes derived from pluripotent stem cells (PSC-CMCs) have emerged as new models for studying different aspects of cardiac disease. Despite the great potential of PSC-CMCs as cardiac model systems, they also have some serious drawbacks that need to be overcome before becoming a practical alternative to the use of laboratory animals. Both the generation of PSCs and the production of more or less homogeneous populations of cardiomyocytes from these cells is a laborious, time-consuming and costly endeavor that requires properly timed treatment of the starting material with various cocktails of expensive growth factors and small molecules. Moreover, PSCs greatly differ in their ability to differentiate into cardiomyocytes and typically yield phenotypically heterogeneous populations of immature cardiac muscle cells. The latter feature is limiting the use of PSC-CMCs in studying cardiac physiology and pathology and has, for instance, made the establishment of homogeneous (confluent) monolayers of these cells for cardiac arrhythmia research by high-resolution optical mapping a challenging endeavor.

Immortalization of mammalian primary cells is typically accomplished by endowing them with viral or non-viral genes encoding proteins that trigger cell division and telomere synthesis (also referred to as immortalization genes). In some instances, these immortalization genes have been flanked with recombinase recognition sites to allow their excision following cell amplification thereby facilitating the subsequent redifferentiation of the cells. However, since the recombinase-mediated excision process is never complete, there will always be some cells that have retained the immortalization gene(s) present in the resulting cell population. These cells will gradually “overgrow” the de-immortalized cells. Cardiomyocyte lines have been generated using cardiac muscle cells from rodents and humans as starting material and the simian virus 40 (SV40) large T (LT) antigen to induce cell proliferation (Steinhelper et al., 1990; Jahn et al., 1996; Claycomb et al., 1998; Rybkin et al., 2003; Davidson et al., 2005; Goldman et al., 2006; Zhang et al., 2009). However, as mentioned before, for most parenchymal cell types including cardiomyocytes, proliferation and (terminal) differentiation are mutually exclusive processes. Permanent LT expression, as occurs in the AT-1 (Steinhelper et al., 1990), HL-1 (Claycomb et al., 1998) and AC (Davidson et al., 2005) lines therefore does not allow the generation of homogeneous populations of differentiated cells. For HL-1 cells this problem was partially overcome by culturing them in a highly specialized medium promoting their cardiomyogenic differentiation (White et al., 2004). In order to control LT activity, Jahn et al. (1996) and Goldman et al. (2006) relied on a temperature-sensitive version of the LT antigen (i.e., LT mutant tsA58) to immortalize neonatal rat and human fetal cardiomyocytes, respectively. Culturing of the neonatal rat cardiomyocytes at the non-permissive temperature of 39° C. promoted their cardiomyogenic differentiation but still did not yield excitable cells with well-developed sarcomeres whereas the human fetal cardiomyocytes failed to express cardiac differentiation markers at both the restrictive and permissive temperature. As an alternative method to control LT activity, Rybkin et al. (2003) generated transgenic mice containing a LT expression cassette under the control of cardiac-specific transcriptional regulatory elements of the murine Nkx2.5 gene from which the LT-coding sequence could be excised using Cre recombinase. Cells isolated from the subendocardial tumor-like structures in the hearts of these animals could be serially passaged >50 times without showing obvious signs of senescence. Following removal of the LT-coding sequence by transduction with an adenovirus vector expressing Cre recombinase, the cells developed sarcomeres and some of them produced weak and slow Ca²⁺ transients after electrical stimulation. This, however, only occurred in low-serum medium containing insulin, transferrin and selenium or after ectopic expression of cardiomyogenic factors and did not result in the generation of contractile cells. Zhang et al. (2009) also relied on the Cre-LoxP site-specific recombination system to generate lines of conditionally immortalized neonatal rat ventricular myocytes. Cre-mediated removal of the LT expression module and exposure to cardiomyogenic differentiation conditions did, however, not give rise to excitable or contractile cells.

The aforementioned immortalization strategy in which the immortalization gene is flanked with recombinase recognition sites to allow its excision following cell amplification thereby facilitating re-differentiation of the cells has some inherent disadvantages. Firstly, immortalization and subsequent de-immortalization of the cells requires two consecutive genetic manipulations. Secondly, due to the specific nature of the recombinase-mediated excision reaction, repeated switching between proliferative and differentiated cell states is impractical making de-immortalization essentially an irreversible process. Thirdly, recombinase-mediated excision reactions are never complete. Consequently, after recombinase treatment of a cell population, there will always be a fraction of cells that has retained the immortalization gene. As these cells will have a proliferative advantage, they will gradually “overgrow” the de-immortalized cells in the culture. Although, in theory this problem could be overcome by linking the expression of the immortalization gene to that of a gene allowing negative selection of the uncombined cells, this is not very practical and may last too long to keep the deimmortalized cells healthy and/or differentiated.

### BRIEF SUMMARY

The present disclosure aims to overcome the drawbacks of currently existing immortalization methods and/or the poor differentiation capacity of deimmortalized cell lines generated by currently existing methods for immortalization and redifferentiation of cells or cell lines.

In a first aspect the provide is a vector comprising:
a promoter sequence;
at least one immortalization gene operably linked to the promoter sequence;
a gene coding for an inducible regulator operably linked to the promoter sequence; and
a response element for the inducible regulator. The promoter is preferably a cell type- or tissue-specific promoter and/or a promoter that is active in a differentiated form of the cell, more preferably a cell type-specific promoter.

Further provided is a genetically modified eukaryotic cell provided with a vector, the vector comprising:
a promoter sequence;
at least one immortalization gene operably linked to the promoter sequence;
a gene coding for an inducible regulator operably linked to the promoter sequence; and
a response element for the inducible regulator.

In a further aspect, the disclosure provides a cell line comprising a plurality of cells according to the disclosure.

In a further aspect, the disclosure provides a kit of parts comprising a cell or cell line according to the disclosure and an agent capable of activating or repressing the inducible promoter.

In a further aspect, the disclosure provides a method for generating one or more conditionally immortalized cells, the method comprising introducing one or more vectors according to the disclosure into one or more eukaryotic cells resulting in one or more genetically modified eukaryotic cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. General overview of a method for reversible immortalization of cells in accordance with the disclosure.

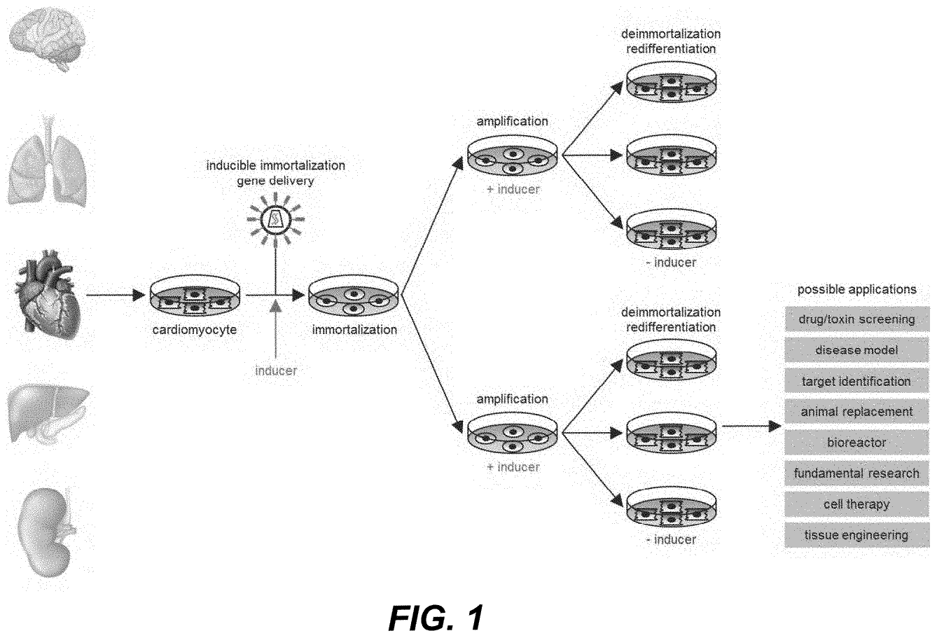

FIGS. 2A and 2B. Example of a vector system used for the reversible cell immortalization in accordance with the disclosure. (FIG. 2A) In the absence of Tc/Dox, immortalization gene expression is repressed facilitating cell differentiation. (FIG. 2B) In the presence of Tc/Dox, immortalization gene expression is induced stimulating cell proliferation. LTR, HIV1 long terminal repeat. Ψ, HIV1 packaging signal. RRE, HIV1 Rev-responsive element. cPPT, HIV1 central polypurine tract and termination site. TRS, transcriptional regulator sequence (i.e., promoter), IRES, encephalomyocarditis virus internal ribosome entry site. tTR, coding sequence of the hybrid Tc-controlled transcriptional repressor TetR-KRAB (Deuschle et al., 1995). TRE, Tc-responsive promoter element consisting of 7 repeats of a 19-nucleotide tetO sequence.

FIG. 3. Conditional immortalization of rat neonatal atrial cardiomyocytes (naCMCs). (Panel A) Scheme of the procedure used for the reversible immortalization, amplification and subsequent redifferentiation of naCMCs. A, atrium. Ori, bacterial origin of replication. Amp^{R}, Escherichia coli ß-lactamase gene. MHCK7, chimeric striated muscle-specific promoter (Salva et al., 2007). LT, coding sequence of the temperature-sensitive mutant LT protein tsA58 (Loeber et al., 1989). WHVPRE, woodchuck hepatitis virus posttranscriptional regulatory element. For an explanation of the other abbreviations, see the legend of FIGS. 2A and 2B. (Panel B) Bright-field images of a single cell (day 6) in a naCMC culture showing Dox-dependent clonal expansion (days 7, 9 and 10). (Panels C-E) Analysis by Western blotting (Panels C, D) and immunocytology (Panel E) of SV40 LT protein expression in conditionally immortalized naCMC (iaCMC) cultures before (day 0) and 3, 6, 9 and 12 days after Dox removal.

FIG. 4. Morphology, proliferative activity and apoptosis analysis of iaCMCs. (Panel A) Bright-field images of proliferating iaCMCs (iaCMC-d0), iaCMCs at differentiation day 9 (iaCMC-d9), naCMCs at culture day 1 (naCMC-d1), and naCMCs at culture day 9 (naCMC-d9). (Panel B) Fluorescence images of proliferating iaCMCs and of iaCMCs at different days after Dox removal immunostained for the proliferation marker Ki-67. (Panel C) Graph showing the percentage of Ki-67⁺ cells in proliferating iaCMC cultures and the percentage of Ki-67⁺ cardiomyocytes in 3-day-old naCMC (naCMC-d3) cultures that was not subjected to mitomycin C treatment. Data are presented as meanistandard deviation (SD), N=3. (Panel D) Quantification of cell numbers in iaCMC cultures with or without Dox. (Panel E) Fluoromicrograph of an naCMC-d3 culture that was not subjected to mitomycin C treatment following double immunostaining for Ki-67 and sarcomeric α-actinin. The Ki-67⁺/α-actinin-cells observed in this culture mainly result from uninhibited proliferation of cardiac fibroblasts (data not shown). (Panel F) Fluorescence images of proliferating iaCMCs and of iaCMCs at different days after Dox removal labeled with Alexa Fluor-568-conjugated annexin V to assess externalization of phosphatidylserine as indicator of apoptosis. (Panel G) Fluorescence images of a 9-day-old, mitomycin C-treated naCMC (naCMC-d9) culture labeled with Alexa Fluor-568-conjugated annexin V.

FIGS. 5A-5D. Cardiomyogenic differentiation potential of iaCMCs. (FIGS. 5A, 5C) Analysis by reverse transcription-quantitative polymerase chain reaction (RT-qPCR) of the expression of the cardiac transcription factor genes Nkx2.5, Gata4 and Mef2c, the cardiac sarcomeric protein genes Actn2 and Myh6 (FIG. 5A), the “atrial” genes Nppa and Myl7 and the “ventricular” gene Myh7 (FIG. 5C) in naCMCs and rat neonatal ventricular cardiomyocytes (nvCMCs) at day 9 of culture and in iaCMCs on the indicated days of differentiation. mRNA levels are expressed relative to those in 9-day-old naCMC (naCMC-d9) cultures that were set at 1. Data are presented as mean±SD, N=3. The low amounts of Myh7 transcripts detected in naCMC samples are most likely due to the presence of contaminating nvCMCs in these samples. Alternatively, naCMCs display low Myh7 gene expression. (FIGS. 5B, 5D) Fluorescence images of proliferating iaCMCs and of iaCMCs at different days after Dox removal immunostained for sarcomeric α-actinin (red, FIG. 5B) or the atrial regulatory myosin light chain MLC2a (green, FIG. 5D). naCMCs and nvCMCs at culture day 9 served as positive and negative control for the MLC2a immunostaining, respectively. Cell nuclei have been visualized by staining with Hoechst 33342 (blue).

FIG. 6. Analysis by RT-qPCR of the expression of the cardiac ion channel genes Scn5a (Panel A), Cacna1c (Panel B), Kcnj3 (Panel C), Kcnj5 (Panel D), Kcnj11 (Panel E), Kcnd3 (Panel F), Kcnh2 (Panel G) and Kcnj2 (Panel H), and the cardiac Ca²⁺-handling protein genes Ryr2 (Panel I) and Atp2a2 (Panel J) in naCMCs and nvCMCs at day 9 of culture and in iaCMCs on the indicated days of differentiation. mRNA levels are expressed relative to those in 9-day-old naCMC (naCMC-d9) cultures that were set at 1. Data are presented as mean±SD, N=3.

FIGS. 7A-7F. Electrophysiological properties of iaCMCs. (FIGS. 7A-7E) Single-cell patch-clamp analysis of iaCMCs and naCMCs. Typical total membrane current traces (a) and action potentials (APs; b) of naCMCs (FIG. 7A), excitable iaCMCs (FIG. 7B) and inexcitable iaCMCs (FIG. 7C). The small inward current observed in (FIG. 7C) corresponds to the input stimulus. (FIG. 7D) Graph showing the percentage of excitable iaCMCs at 3, 6, 9 and 12 days of differentiation. N=9, 9, 7 and 8 for iaCMC-d3, iaCMC-d6, iaCMC-d9 and iaCMC-d12, respectively. (FIG. 7E) Graph showing the cell capacitance of iaCMCs at 3, 6, 9 and 12 days of differentiation in comparison to that of naCMCs at culture day 2-4. Data are presented as mean±SD, N=10 for the naCMCs and N=9, 9, 7 and 8 for iaCMC-d3, iaCMC-d6, iaCMC-d9 and iaCMC-d12, respectively. (FIG. 7F) Fluorescence images of proliferating iaCMCs and of iaCMCs at different days after Dox removal immunostained for Cx43 (green) showing its differentiation-dependent accumulation at the sarcolemma in gap junctional plaques. As positive controls for the Cx43 immunostaining, 9-day-old naCMC and nvCMC cultures were included. Hoechst 33342 (blue) staining shows cell nuclei. (FIGS. 7G-7I) Optical voltage mapping of confluent iaCMC and 9-day-old naCMC (naCMC-d9) monolayers following 1-Hz electrical point stimulation. (FIGS. 7G, 7H) Typical activation maps (FIG. 7G; isochrones are separated by 6 ms) and optical signal traces (FIG. 7H) of iaCMC cultures at different days after Dox removal showing an increase in conduction velocity (CV) and a decrease in AP duration (APD) with advancing iaCMC differentiation. Quantification of CV (FIG. 7I) and APD at 30 and 80% repolarization (APD₃₀ FIG. 7J and APD₈₀ [FIG. 7K], respectively) in naCMC-d9 cultures and in iaCMC cultures at 3, 6, 9 and 12 days of differentiation. Data are presented as mean±SD, N=14.

FIG. 8. Characterization by optical voltage mapping and 1-Hz electrical point stimulation of confluent monolayers of iaCMC clones #1 (Panels A-C) and #6 (Panels D-F). Typical activation maps (Panels A, D; isochrones are separated by 6 ms) and optical signal traces (Panels B, E) acquired a different days after Dox removal showing an increase in CV and a decrease in APD with advancing iaCMC differentiation. (Panels C, F) Quantification of CV, APD₃₀ and APD₈₀ at 3, 6 and 9 days of iaCMC differentiation. Data are presented as mean±SD, N=12.

FIG. 9. Comparison by optical voltage mapping and 1-Hz electrical point stimulation of the electrophysiological properties of confluent monolayers of iaCMCs at day 9 of differentiation (iaCMC-d9) with those of HL-1 cells. Typical activation maps (Panel A; 6-ms isochrone spacing) and optical signal traces (Panel C) showing a much slower CV (Panel B) and APD (Panel C) for the HL-1 cells as compared to the iaCMC-d9. Data are presented as mean±SD, N=5.

FIG. 10. Suitability of iaCMCs as in vitro atrial fibrillation (AF) model assessed by optical voltage mapping. (Panels A, F) Typical optical signal traces of mock- (Panel A) and tertiapin (Panel F)-treated iaCMCs at day 9 of differentiation (iaCMC-d9) after high-frequency (10-50 Hz) electrical point stimulation. (Panel B) Activation maps (6-ms isochrone spacing) of iaCMC-d9 cultures showing 1 (left) or 2 (right) reentrant circuits. (Panels C-E) Inducibility of reentry (Panel C), arrhythmia complexity (Panel D) and quantification of rotor frequency (Panel E), in iaCMC cultures at 3, 6, 9 and 12 days of differentiation. (Panel G) Typical optical AP records of mock- (red) and tertiapin (blue)-treated iaCMC-d9 following 1-Hz pacing. (Panel H) Quantification of APD₃₀ and APD₈₀ of mock- and tertiapin-treated iaCMC-d9. Data are presented as mean±SD, N=10.

FIG. 11. Suitability of iaCMCs as in vitro model to assess the I_{Kr}-blocking potential of drugs by optical voltage mapping. (Panel A) Immunofluorescence analysis of Kᵥ11.1 protein expression in iaCMC-d6 cultures and in 9-day-old naCMC cultures. (Panels B-E) Optical signal traces (Panel B), quantitative analyses of APD₄₀ and APD₉₀ (Panel C), activation maps (Panel D) and CV (Panel E) following 1-Hz pacing of iaCMC-d6 cultures exposed for 30 minutes to 100 nM astemizole or vehicle (control). Data are presented as mean±SD, N=6.

FIG. 12. Comparison by optical voltage mapping and 1-Hz electrical point stimulation of the electrophysiological properties of confluent 9-day-old cultures of naCMCs, Dox-deprived eGFP-labeled iaCMCs (eGFP-iaCMCs) and 50% naCMCs-50% eGFP-iaCMCs. (Panel A) Bright-field images (upper panel) and fluorescence images (lower panel) of each culture type. (Panel B) Fluorescence image of 9-day-old naCMC/eGFP-iaCMC co-culture immunostained for Cx43 (red) showing its presence at interfaces between iaCMCs (green) and naCMCs. Cell nuclei have been visualized by staining with Hoechst 33342 (blue). (Panels C and D) Activation maps (Panel C; isochrones are separated by 6 ms) and CV quantification (Panel D) reveal no significant different in the speed of AP propagation between the 3 culture types. Data are presented as mean±SD, N=6.

FIG. 13. Structural integration of iaCMCs into neonatal rat atrium. (Panel A) Experimental design to assess the engraftment and cardiomyogenic differentiation potential of iaCMCs following their injection into neonatal rat atrium. A, atrium. (Panel B) Analysis by confocal laser scanning fluorescence microscopy of the presence and phenotype of eGFP-labeled iaCMCs following double immunostaining for eGFP (green) and sarcomeric α-actinin (red) and labeling of cell nuclei with Hoechst 33342 (blue). The 2 small panels in the middle row represent higher magnifications of the upper 2 eGFP⁺ cells in the adjacent larger panel. A series of YZ reconstructions of the eGFP⁺ cell-rich zone in this panel (area demarcated by white lines) is shown in the bottom panel. (Panel C) Sarcomere lengths of endogenous naCMCs and of iaCMCs that underwent cardiomyogenic differentiation following intra-atrial injection. Data are presented as mean±SD, N=14.

FIG. 14. Transient transfection of iaCMCs with plasmid DNA. (Panel A) Direct fluorescent images of iaCMCs immediately prior to lipofection with the eGFP-encoding mammalian expression plasmid pU.CAG.eGFP (left panel) and following maintenance for 2 or 9 days in culture medium without Dox. (Panel B) Quantification, by flow cytometry, of the percentage of eGFP⁺ cells in the cultures shown in (Panel A). (Panel C) Fluoromicrograph of an iaCMC culture that was transfected with pU.CAG.eGFP, subsequently maintained for 9 days in Dox-free culture medium and finally immunostained for sarcomeric α-actinin. (Panel D) Activation maps (6-ms isochrone spacing) of untransfected (−pU.CAG.eGFP) and pU.CAG.eGFP-transfected (+pU.CAG.eGFP) iaCMC cultures kept for 9 days in Dox-free culture medium.

FIG. 15. Activity of MHCK7 promoter in proliferating iaCMCs and in iaCMCs at different days after initiation of cardiomyogenic differentiation. (Panel A) Map of lentiviral vector shuttle plasmid pLV.MHCK7.eGFP eGFP, eGFP-coding sequence. WHVoPRE, optimized version of the WHVPRE (Schambach et al., 2006), SIN-LTR, 3′ long terminal repeat containing a 400-bp deletion in unique region 3 rendering the corresponding lentiviral vector self-inactivating (Zufferey et al., 1998). For an explanation of the other abbreviations, see the legend of FIGS. 2A and 2B. (Panel B) Fluoromicrographs of LV.MHCK7.eGFP-transduced iaCMCs showing an increase in MHCK7 promoter activity as evinced by an increase in eGFP fluorescence with advancing iaCMC differentiation.

FIG. 16. Comparison by optical voltage mapping and 1-Hz electrical point stimulation of the electrophysiological properties of “different-aged” iaCMCs at day 9 of differentiation (iaCMC-d9). After being cryopreserved, the cells went through 25, 40 or 55 passage doublings (PD) prior to Dox removal. CV, APD₃₀ and APD₈₀ did not significantly differ between iaCMC-d9 of different PD. Data are presented as mean±SD, N=4.

FIG. 17. Comparison by optical voltage mapping and 1-Hz electrical point stimulation of APD₃₀ and APD₈₀ of naCMCs and nvCMCs in 9-day-old cultures with those of iaCMCs at day 9 of differentiation showing that the nvCMCs have much longer APDs than the atrial cardiomyocytes. Data are presented as mean±SD, N=12.

FIG. 18. Experimental setup of the transcriptome analysis of conditionally immortalized neonatal rat atrial myocytes (iAM-1 cells) undergoing one cycle of cardiomyogenic differentiation and dedifferentiation. Proliferating iAM-1 cells (day 0) were first cultured for 9 days in the absence of Dox to allow them to differentiate into excitable and contractile cells and subsequently for 9 days in the presence of 100 ng/ml Dox causing the cells to dedifferentiate and to regain a proliferative phenotype. At the indicated stages, total RNA from 4 independent samples was extracted for whole transcriptome shotgun sequencing (RNA-Seq; ≥2×10⁷ 150-bp paired-end reads/sample).

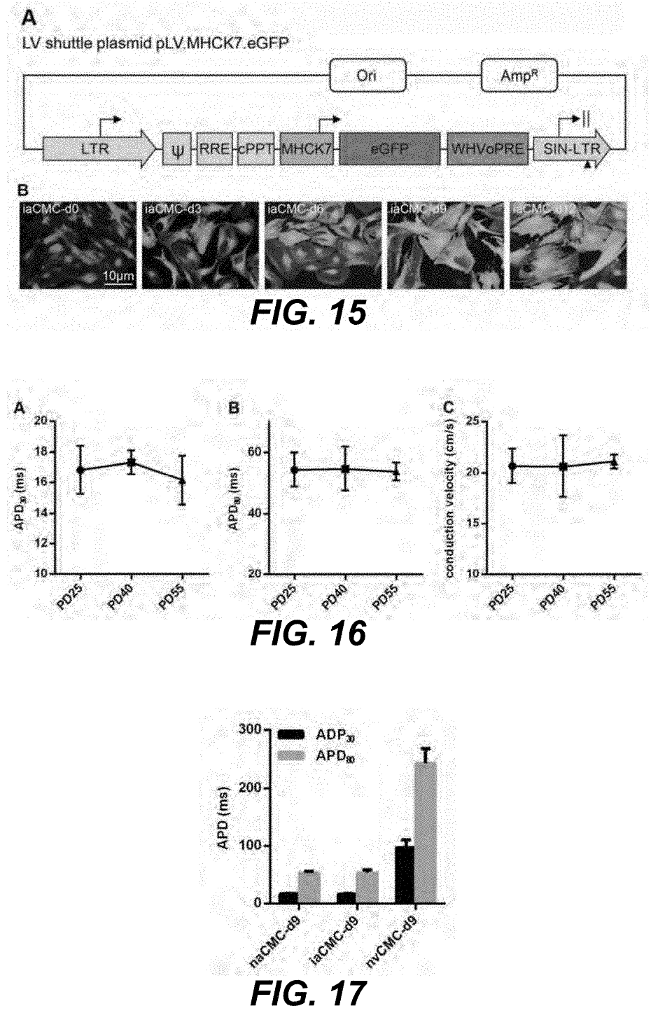

FIG. 19. Relative contribution of the 7 most abundant transcripts to the entire transcriptome of iAM-1 cells at different stages of cardiomyogenic differentiation and dedifferentiation. Cardiomyogenic differentiation causes increases in the expression of the sarcomeric protein-encoding genes Actc1, Tpm1, Myh6 and Tnnt2, of the gene for sarcoplasmic/endoplasmic reticulum Ca²⁺ transporting ATPase 2 (Atp2a2) and of the gene encoding atrial natriuretic peptide (Nppa). Subsequent induction of proliferation leads to a drop in the expression of these genes.

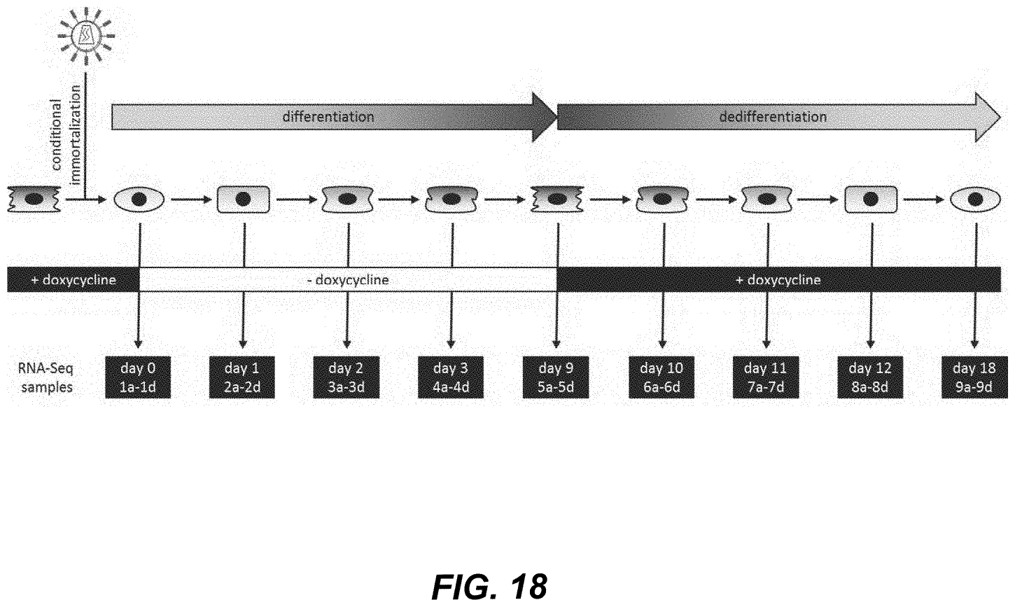

FIG. 20. Evaluation of the similarity between the RNA samples used for the RNA-Seq study of iAM-1 cells undergoing one cycle of cardiomyogenic differentiation and dedifferentiation by principal component analysis. PC1 and PC2 represents 60% and 20% of the observed variation in transcript counts, respectively. Different RNA samples from the same stage have very similar transcriptomes and iAM-1 cells that underwent one cycle of differentiation and dedifferentiation (samples 9a-9d) strongly resemble the starting material (samples 1a-1d).

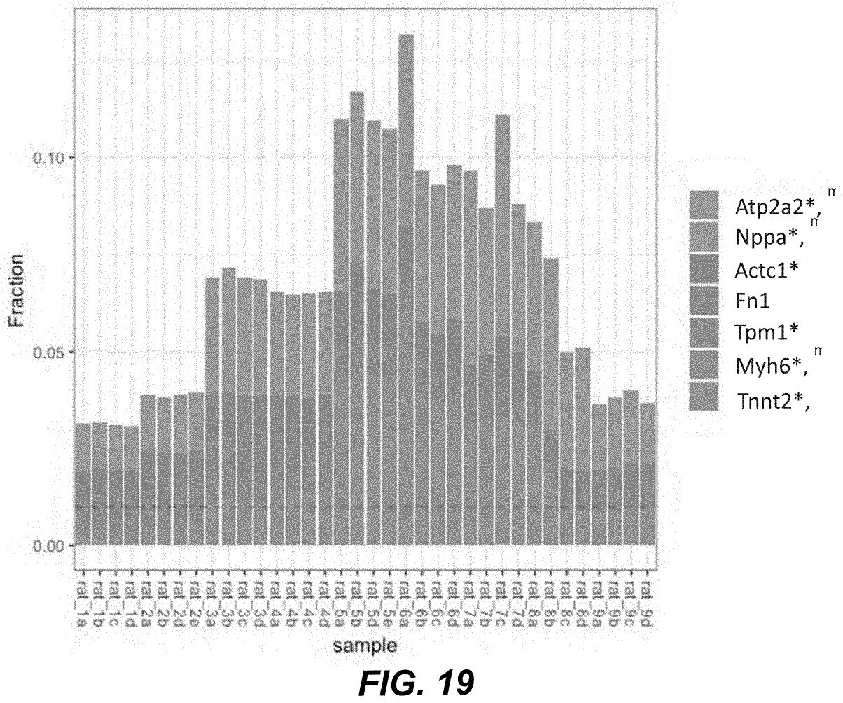

FIG. 21. Clonal expansion during a period of ±2 weeks of right atrial cells from a 21-week-old human fetus following transduction with LV.iMHCK7.LT-WT and maintenance in the presence of 100 ng/ml doxycycline (Dox).

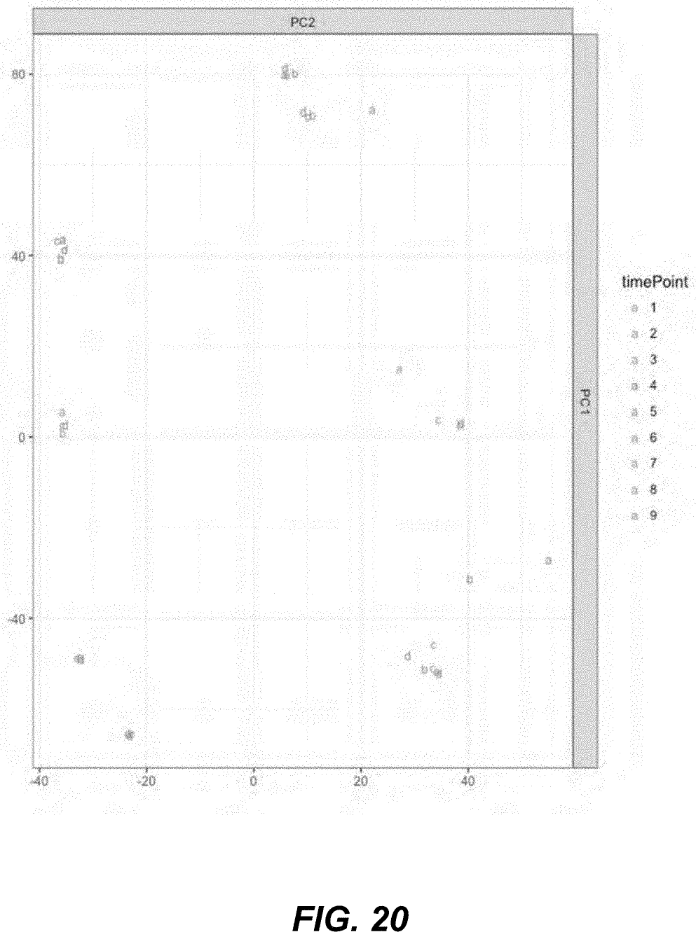

FIG. 22. Phase contrast images of conditionally immortalized human fetal atrial cardiomyocyte clone 7L#6 during proliferation (left image, +Dox) and following cardiomyogenic differentiation (i.e., at 9 days after Dox removal; right image, −Dox). The starting material was derived from the left atrium of an 18-week-old human fetus.

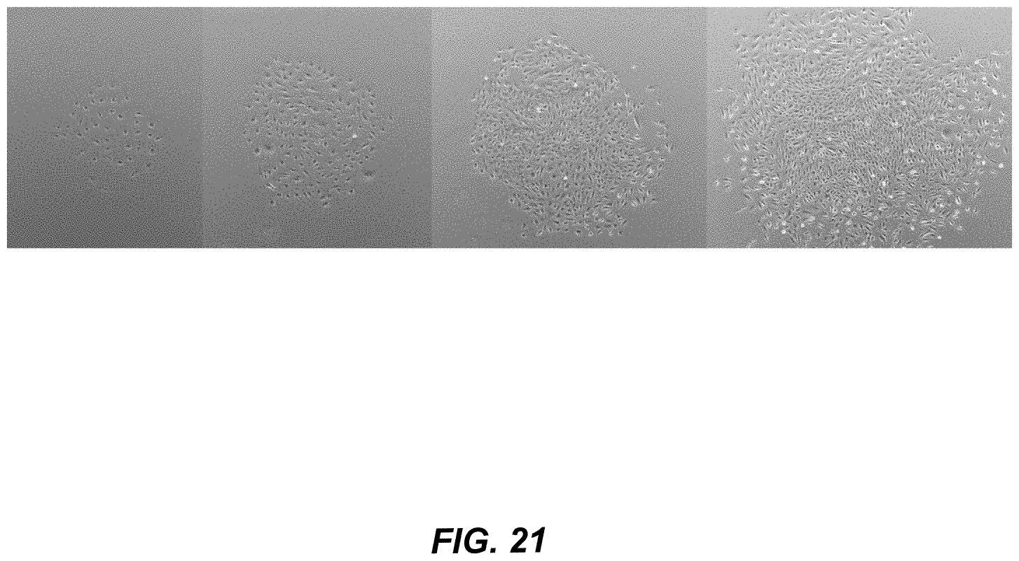

FIG. 23. Fluoromicrographs of conditionally immortalized human fetal atrial cardiomyocyte clone 7L#4 during proliferation (upper panel, +Dox) and following cardiomyogenic differentiation (i.e., at 9 days after Dox removal; lower panel, −Dox). The starting material was derived from the left atrium of an 18-week-old human fetus. The cells were stained with the DNA-binding dye Hoechst 33324 (left column), with antibodies directed against the proliferation marker Ki-67 (middle column) or with antibodies recognizing the large T (LT) oncoprotein of simian virus 40.

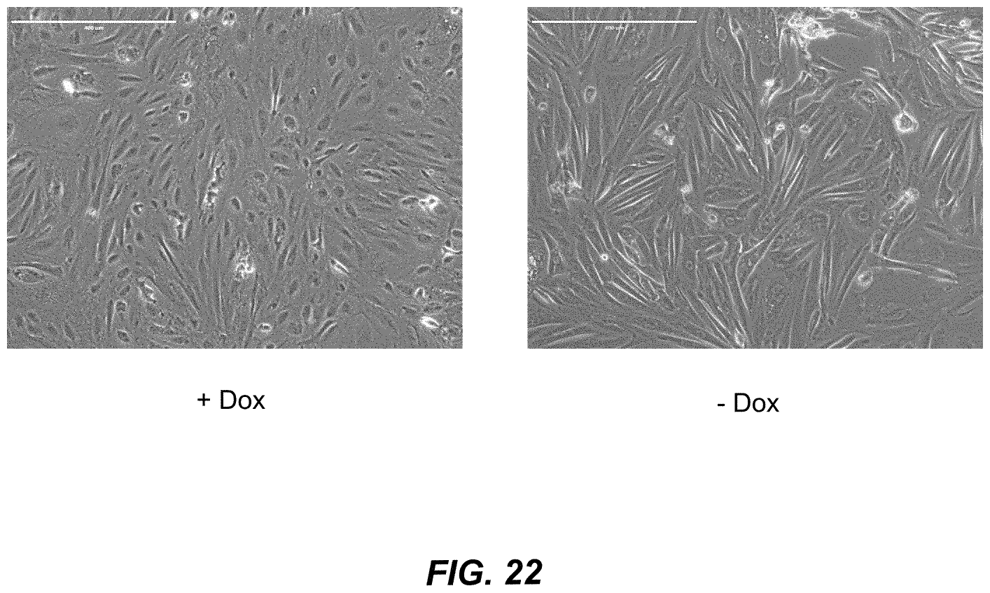

FIG. 24. Fluoromicrographs of a primary human fetal left atrial cardiomyocyte (pAM; left image) and of clone 7L#4 during proliferation (middle image, +Dox) and following cardiomyogenic differentiation (i.e., at 9 days after Dox removal; right image −Dox). The starting material was derived from the left atrium of an 18-week-old human fetus. The cells were stained with the DNA-binding dye Hoechst 33324 (blue) and with antibodies directed against the sarcomeric protein cardiac troponin T.

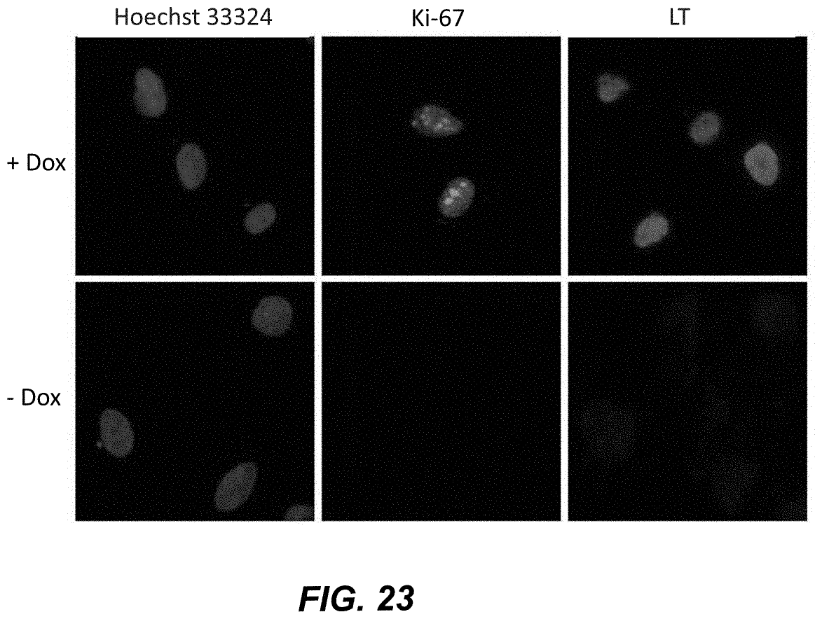

FIG. 25. Assessment, by optical voltage mapping, of the electrophysiological properties of conditionally immortalized human fetal atrial cardiomyocyte clones 7L#12 and 7L#4 following cardiomyogenic differentiation (i.e., at 12 days after Dox removal). The starting material was derived from the left atrium of an 18-week-old human fetus. (Panels A, B) Typical optical voltage trace (Panel A) and activation map (Panel B) of clone 7L#12 monolayers following 1-Hz electrical point stimulation. The spacing between isochrones is 12 ms. (Panel C) Action potential duration at 80% repolarization (APD80) and conduction velocity (CV) of monolayer cultures of clones 7L#12 (n=3) and 7L#4 (n=6) analyzed by optical voltage mapping (Panel D) Effect of the K_{ATP} channel opener P1075 on the APD80 and CV of an electrically paced clone 7L#12 monolayer.

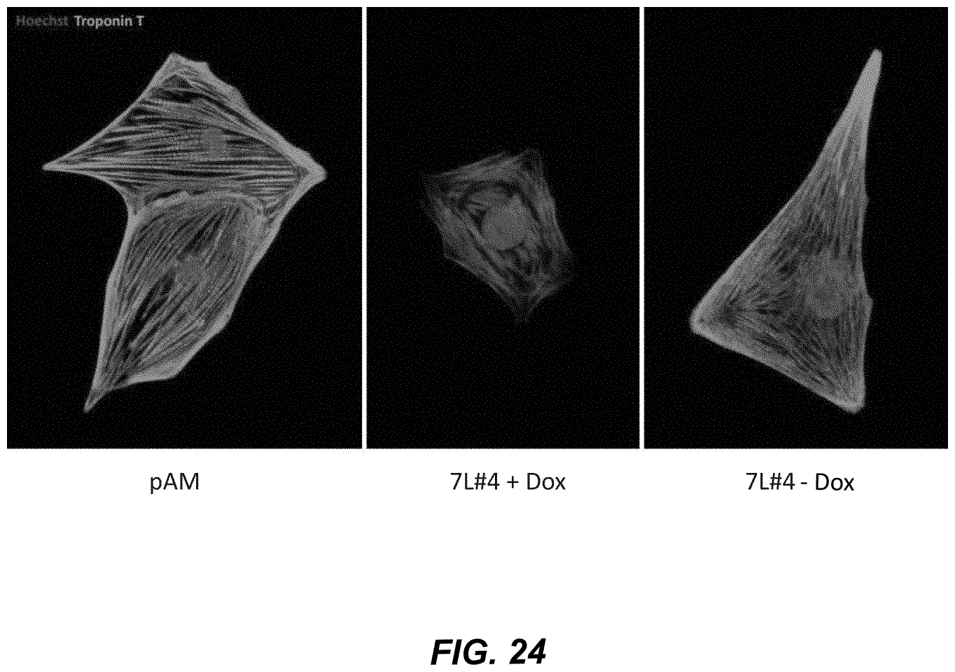

FIG. 26. Current clamp record of a spontaneously active clone 7L#4 cell cultured for 16 days in Dox-free medium.

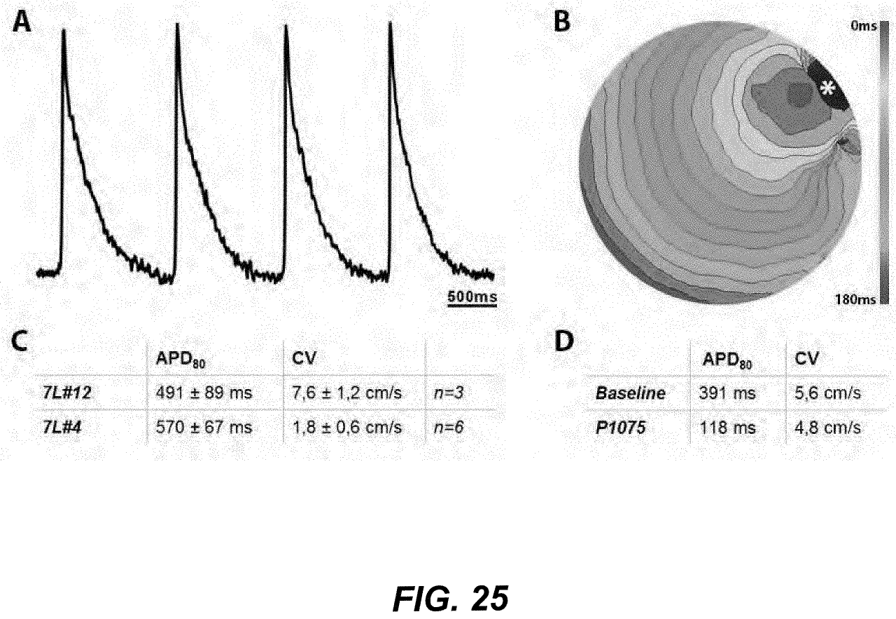

FIGS. 27A-27E. (FIG. 27A) Overview of the method to generate conditionally immortalized epicardial mesothelial cells (iEPDCs). (FIG. 27B) Microscopic images of mock-(left) and LV.ihEEFlal.LT-tsA58 (right)-transduced EPDC cultures initially containing equal numbers of cells. (FIGS. 27C, 27D) Immunocytological (FIG. 27C) and Western blot (FIG. 27D) analysis of LT expression in cultures of iEPDCs kept in the presence of Dox (+dox) or for the indicated days in its absence (−dox). (FIG. 27E) Proliferation of iEPDCs cultured in regular growth medium supplemented with vehicle (PBS), transforming growth factor β1 (TGFβ1), the TGFβ type I receptor inhibitor SB431542 (SB), Dox (dox), dox+TGFβ1 or dox+SB. LV.ihEEFlal.LT-tsA58 is identical to LV.iMHCK7.LT-tsA58 except for the presence of the human EEF1A1 promoter instead of the MHCK7 promoter to drive LT-tsA58 expression. hEEF1α1, human EEF1A1 promoter. tTS, coding sequence of the hybrid tetracycline-controlled transcriptional repressor TetR-KRAB (previously abbreviated tTR). For an explanation of the other abbreviations, see the legends of FIGS. 2A, 2B and 3.

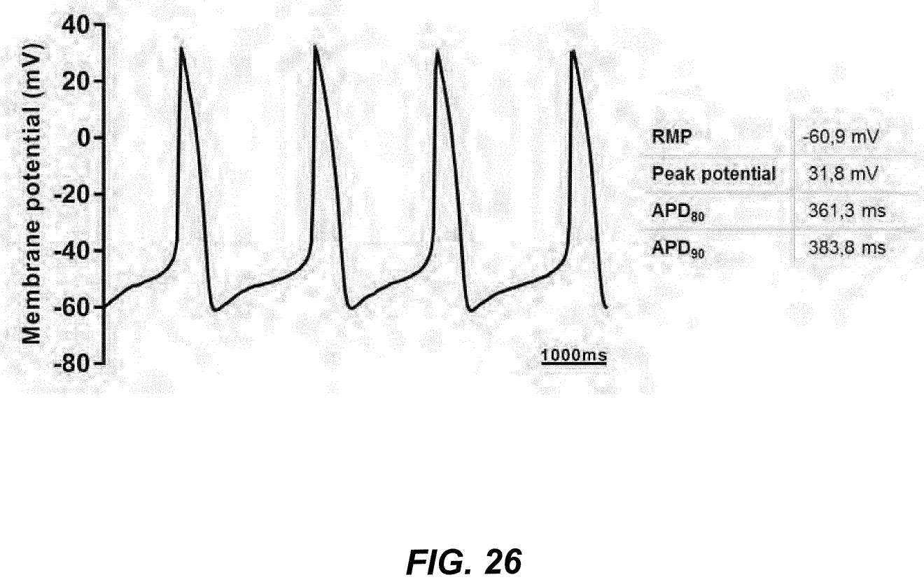

FIG. 28. (Panels A-F) Phase contrast microscopy of iEPDC cultures treated according to the added scheme showing evidence of epithelial-mesenchymal and mesenchymal-epithelial transition.

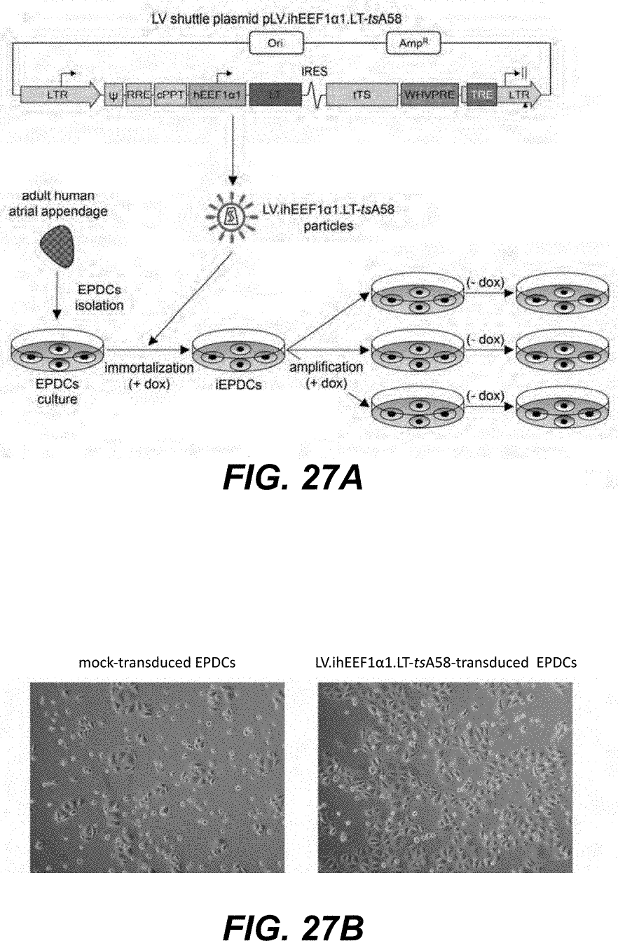

FIG. 29. Conditional immortalization of brown preadipocytes (BPAs). (Panel A) Scheme of the procedure used for the reversible immortalization, amplification and subsequent redifferentiation of BPAs. For an explanation of the abbreviations see the legend of FIGS. 2A and 2B, 3, and 27A-27E. (Panel B) Growth curves of primary BPAs (nBPAs) and of LT-transduced/expressing BPAs (tBPAs). While nBPAs stopped dividing/became senescent around passage doubling (PD) 18, tBPAs continued to proliferate unabated for at least 60 PDs. (Panel C) Comparison, by bright-field microscopy, of 4-day-old cultures of tBPAs maintained in the absence or presence of 100 ng/ml Dox (dox) and of PD18 BPAs. (Panels D, E) Analysis by Western blotting (Panel D) and immunocytology (Panel E) of LT protein expression in nBPAs and in tBPAs cultured in medium containing 100 ng/ml Dox. GAPDH, glyceraldehyde 3-phosphate dehydrogenase. The blue fluorescence corresponds to cell nuclei stained with Hoechst 33342.

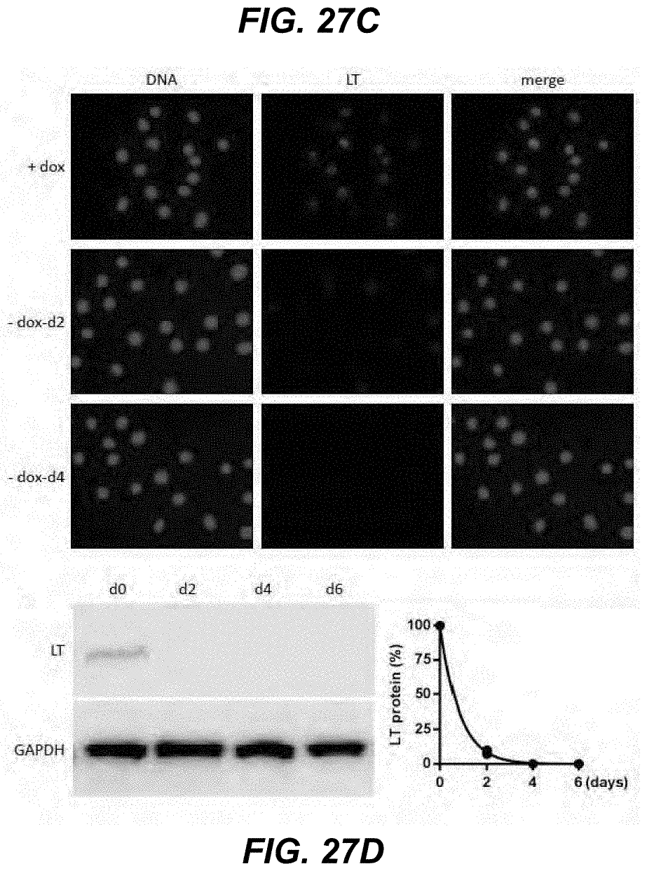

FIG. 30. Dox-dependent LT expression and proliferation ability of murine tBPA clone 6 at 40 and 100 PDs. (Panels A-C) Analysis by immunocytology (Panel A) and Western blotting (Panels B, C) of LT protein expression in PB40 and PB 100 clone 6 tBPAs maintained for 0, 2, 4, 6 and 8 days in Dox-free medium. (Panel D) Proliferation rate of PB40 and PB 100 clone 6 tBPAs cultured in the absence or presence of 100 ng/ml Dox.

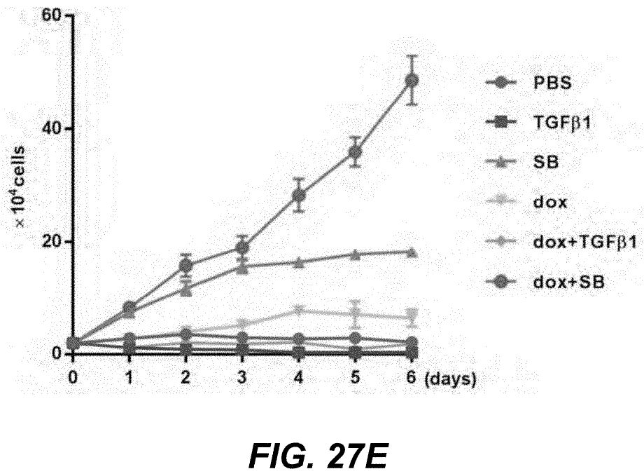

FIG. 31. Bright field images of murine clone 6 and 7 tBPAs cultured for the indicated days in Dox-free adipogenic differentiation medium.

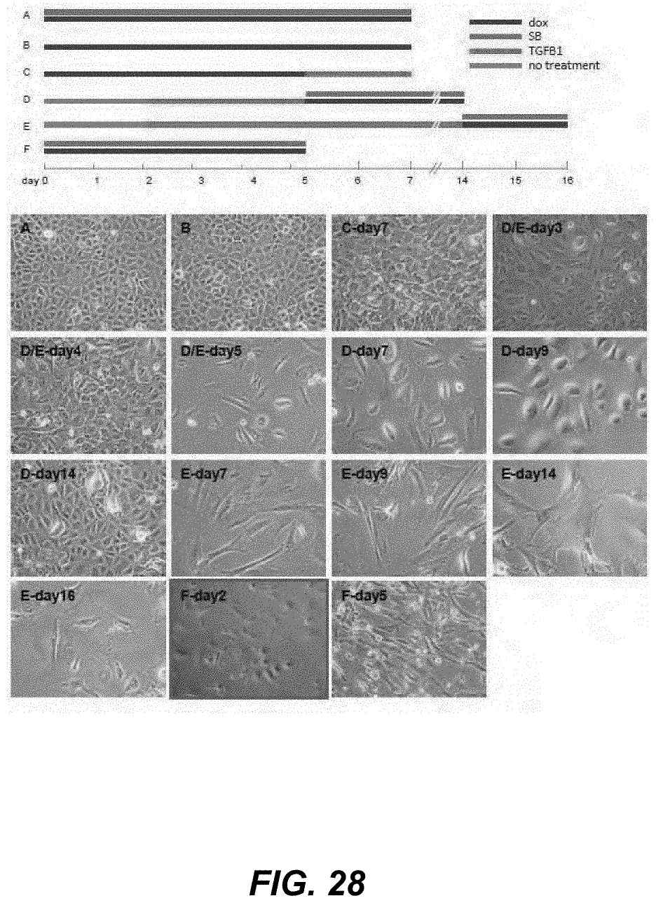

FIG. 32. Analysis by RT-qPCR of the expression of brown fat cell markers by murine clone 6 and 7 tBPAs cultured for the indicated days in Dox-free adipogenic differentiation medium.

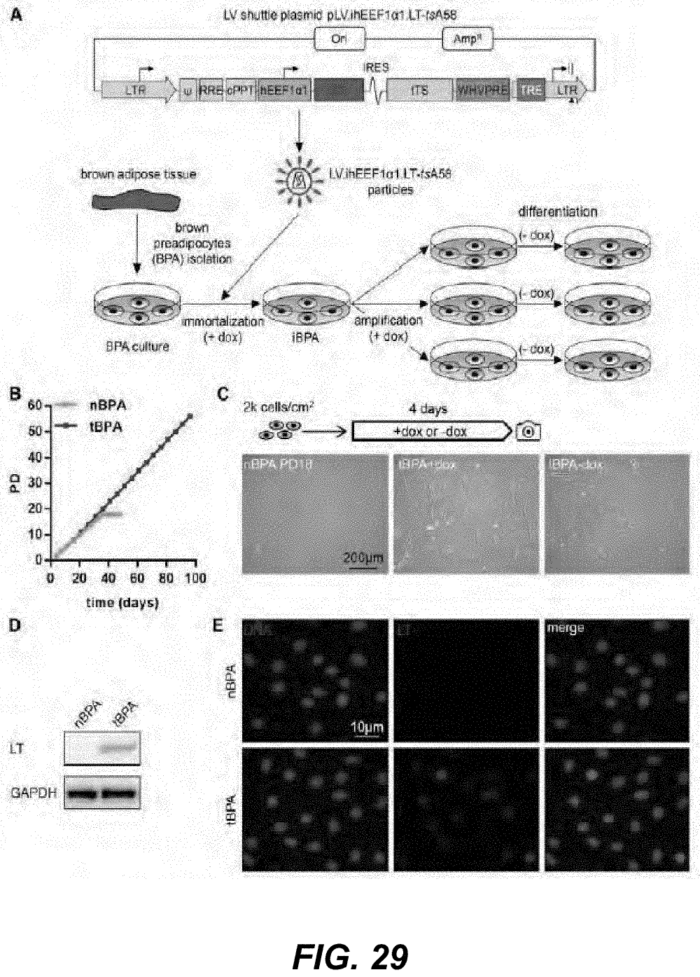

FIG. 33. Adipogenically differentiated murine clone 6 and 7 tBPAs show increased glycerol production and Ucp1 transcript levels following treatment with the broad spectrum adrenoreceptor agonist noradrenaline (NA) or with the 33 adrenoreceptor agonist CL316243.

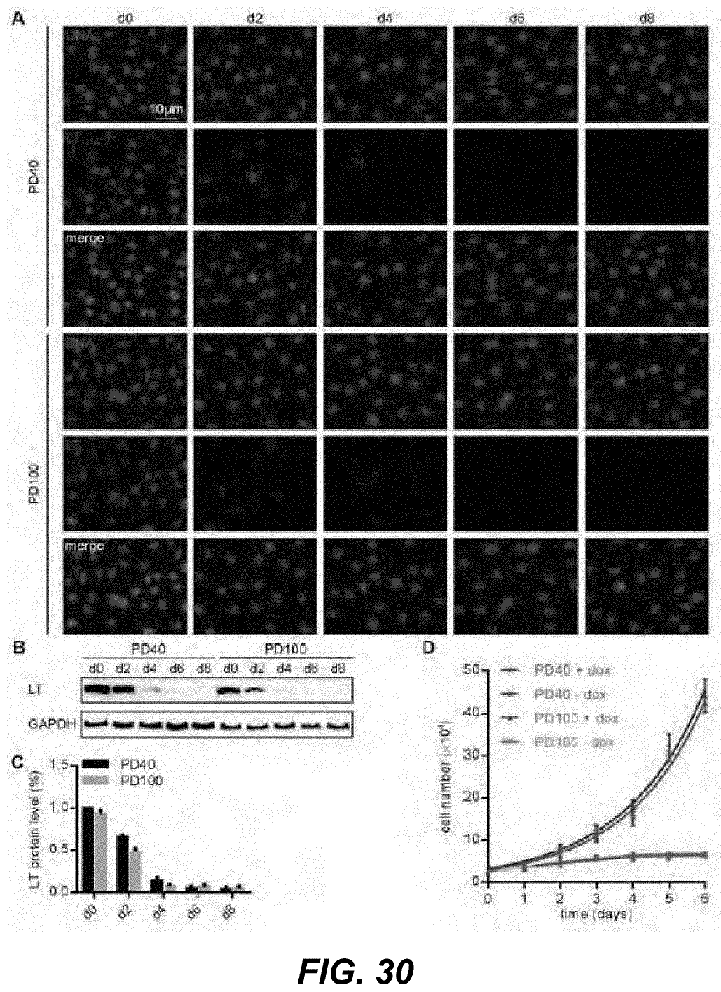

FIG. 34. Oxygen consumption of adipogenically differentiated murine clone 6 and 7 tBPAs to consecutive treatments with the 33 adrenoreceptor agonist CL316243 or vehicle, the ATP synthase inhibitor oligomycin, the mitochondrial oxidative phosphorylation uncoupler/protonophore carbonyl cyanide-4-(trifluoromethoxy)phenylhydrazone (FCCP), and a mixture of the mitochondrial complex I inhibitor rotenone and the ubiquinol:cytochrome c oxidoreductase inhibitor antimycin A.

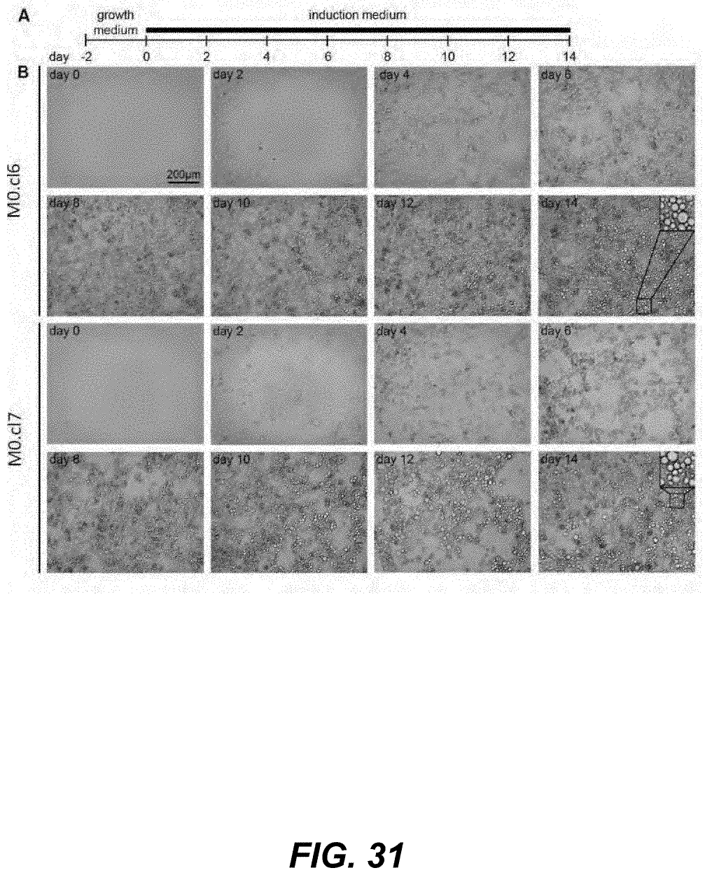

FIG. 35. Bright-field images of human clone 2 and 3 tBPAs at 16 days after the exposure to adipogenic differentiation medium without (regimen 1 and 2) or with (regimen 3) Dox.

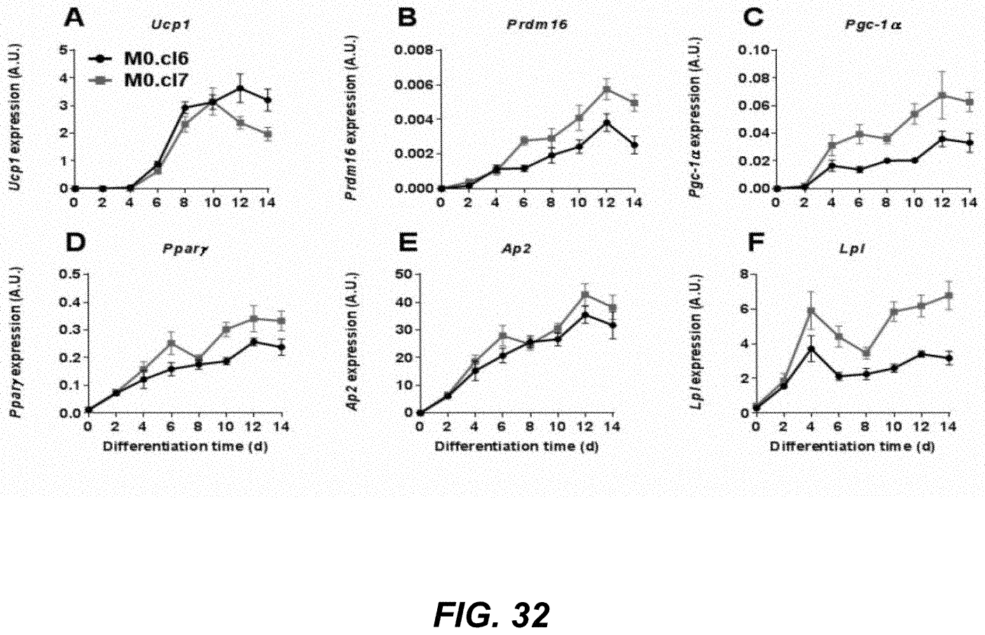

FIG. 36. Bright-field images of human clone 2 and 3 tBPAs cultured for the indicated days in Dox-free regimen 2 adipogenic differentiation medium.

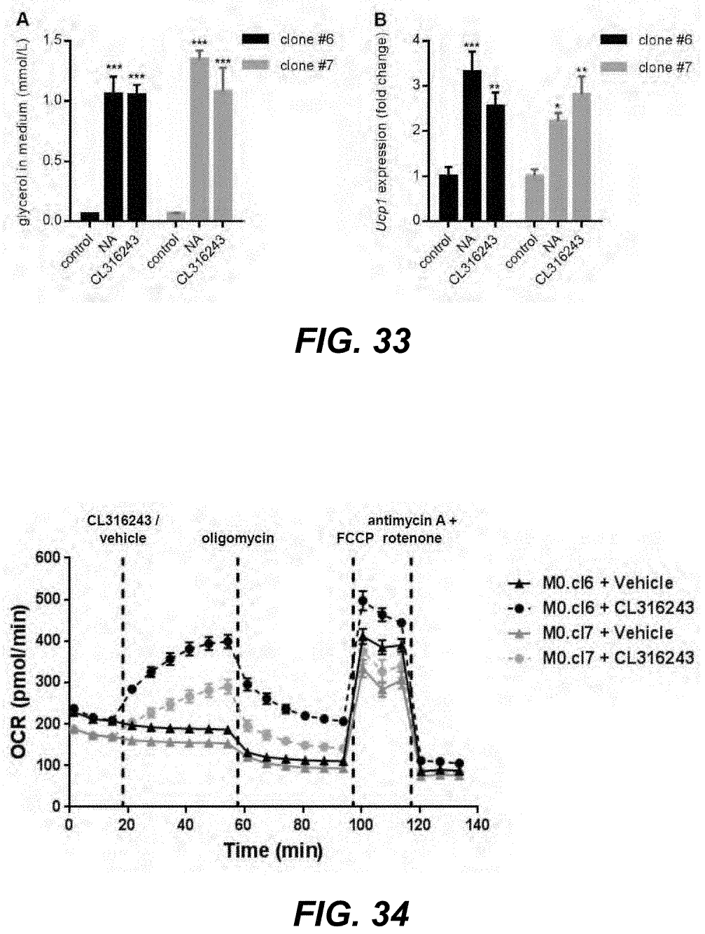

FIG. 37. Analysis by RT-qPCR of the expression of brown fat cell markers UCP1, PRDM16 and PPARGC1A (also known as PGC-1α) by human clone 2 and 3 tBPAs subjected to adipogenic differentiation regimens 1 (−dox before diff, green line), 2 (−dox from diff, blue line) or 3 (+dox, red line).

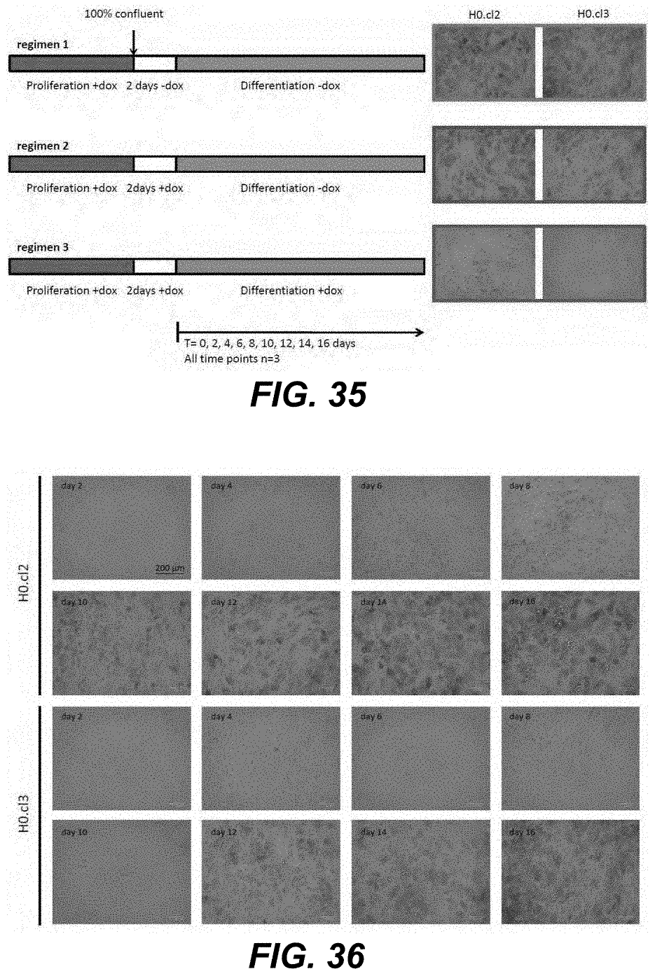

FIG. 38. Analysis by RT-qPCR of the expression of brown fat cell markers PPARG and FABP4 (also known as AP2) by human clone 2 and 3 tBPAs subjected to adipogenic differentiation regimens 1 (green line), 2 (blue line) or 3 (red line).

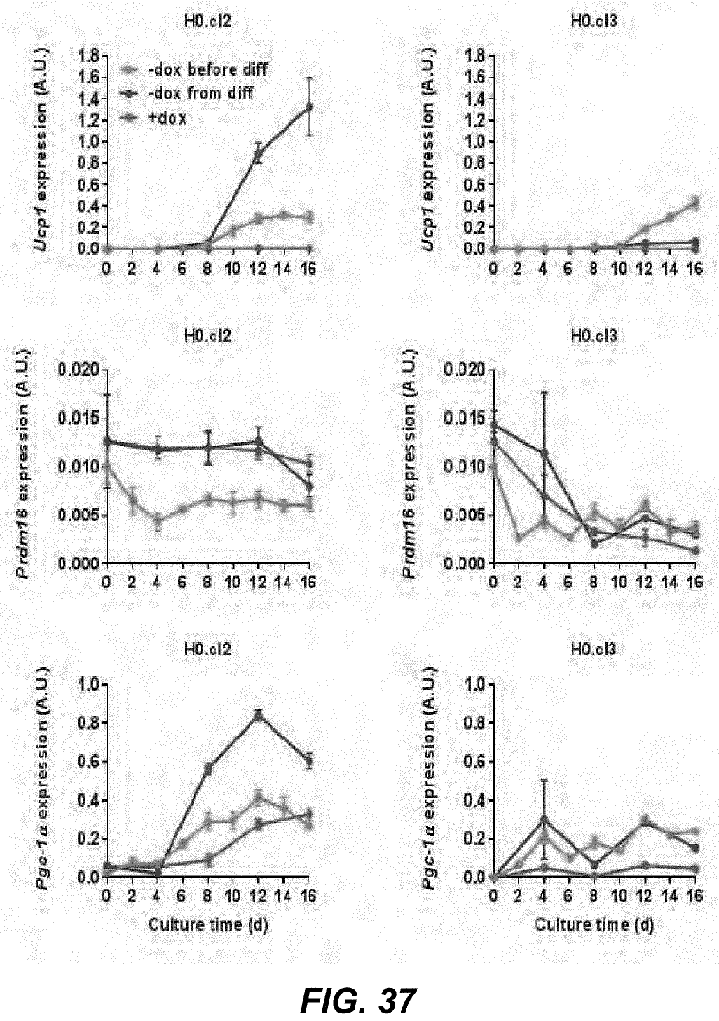

FIG. 39. Analysis by RT-qPCR of the expression of brown fat cell markers by human clone 2 and 3 tBPAs subjected to regime 2 adipogenic differentiation.

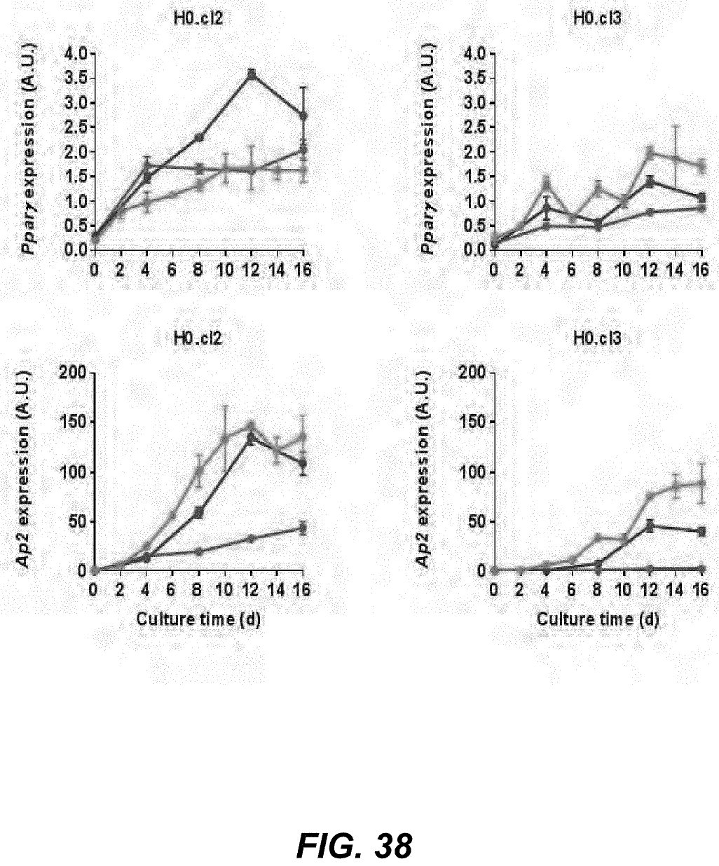

FIG. 40. (Panel A) Glycerol release by adrenergically stimulated human clone 2 tBPAs measured at day 15 of adipogenic differentiation (regimen 2 without or with dexamethasone [Dex]). (Panel B) Glycerol release by adrenergically stimulated human clone 3 tBPAs measured at the indicated days of regimen 2 adipogenic differentiation in the presence of Dex.

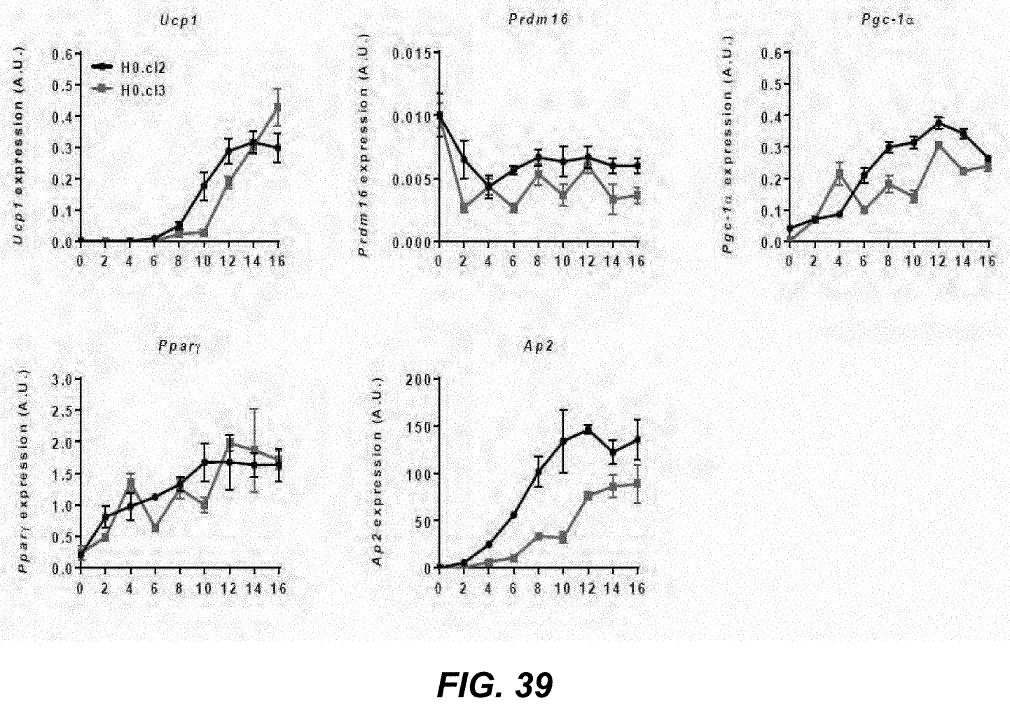

FIG. 41. Glycerol release by adrenergically stimulated tBPA clones derived from 3 different individuals at day 16 of adipogenic differentiation (regimen 1 without Dox).

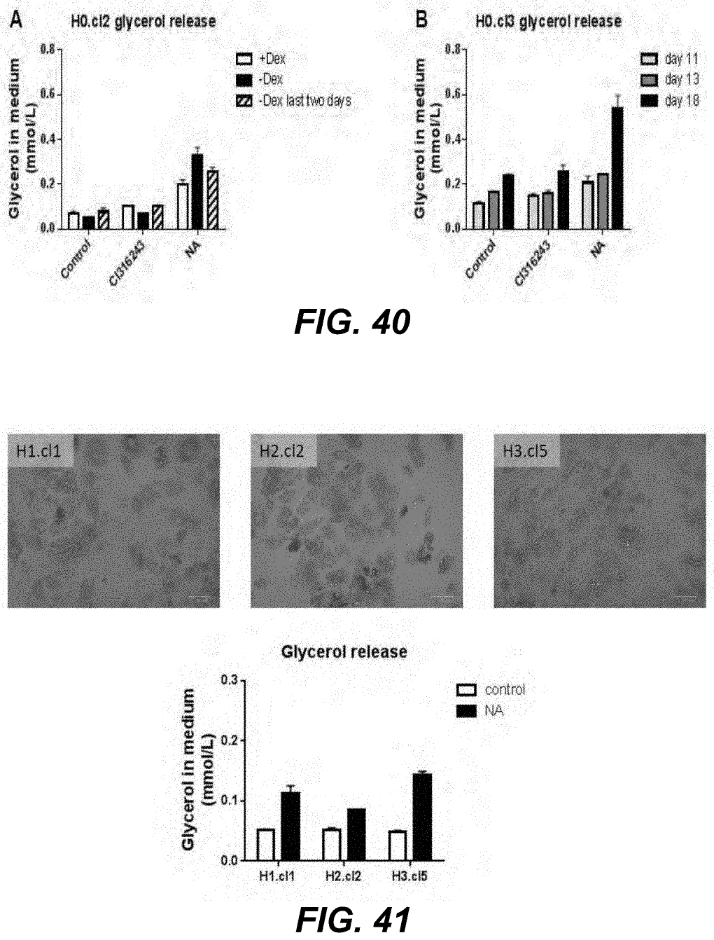

### DETAILED DESCRIPTION

The present inventors have developed a novel method for conditionally and reversibly immortalizing and subsequently re-differentiating cells. It was surprisingly found that with this method conditionally immortalized cardiomyocytes spontaneously and synchronously underwent full cardiomyogenic differentiation following shutdown of immortalization factor production giving rise to cells that very closely resembled the primary cardiomyocytes from which they were derived without the use of complex or expensive cocktails of differentiation factors. Both the extent, i.e., the percentage and the quality, i.e., the functionality/maturity of the cardiomyogenically differentiated cells is without precedent. The reversibly immortalized cells of the disclosure have great potential as models for both fundamental and applied cardiac research and provide easy-to-use and cost-effective alternatives for PSC-derived differentiated cells. In particular, the obtained cells outperform all existing lines of cardiac muscle cells in terms of cardiomyogenic differentiation ability.

In a first aspect the disclosure therefore provides a vector comprising:
a promoter sequence;
at least one immortalization gene operably linked to the promoter sequence;
a gene coding for an inducible regulator operably linked to the promoter sequence; and
a response element for the inducible regulator. Such vector is herein also referred to as a vector according to the disclosure.

Also provided is a genetically modified eukaryotic cell provided with such vector according to the disclosure.

As used herein, the term “vector” refers to a nucleic acid molecule used as a vehicle to introduce nucleic acid sequences into a eukaryotic cell. The nucleic can be DNA or RNA. In a preferred embodiment, the vector according to the disclosure is a DNA molecule. Introduction of a vector into a eukaryotic cell yields a genetically modified cell.

Vectors known to the art that can be used in the disclosure include viral vectors and free nucleic acids, such as plasmids or nucleic acid fragments. In a preferred embodiment, the vector according to the disclosure is a viral vector or a plasmid, more preferably a viral vector. Viral vectors useful in this disclosure include those based on Retroviridae including alpharetro-, betaretro-, deltaretro-, epsilonretro-, gammaretro-, lenti- and spumaviruses, Parvoviridae, Hepadnaviridae, Herpesviridae (e.g., Epstein-Barr virus and human herpesvirus 6), Papillomaviridae, Polyomaviridae (e.g., SV40 and Merkel cell polyomavirus), Adenoviridae, Baculoviridae, Circoviridae and Poxviridae) as well as chimeric viral vectors containing elements of two or more different viruses. The viral vector is preferably a replication-defective viral vector such as an integration-competent lentiviral vector. Suitable viral vectors can be obtained from, amongst others, human immunodeficiency virus (HIV), simian immunodeficiency virus (SIV), equine infectious anemia virus (EIAV), bovine immune deficiency virus (BIV), caprine arthritis encephalitis virus (CAEV), visna virus, Jembrana disease virus (JDV) and feline immunodeficiency virus (FIV). In another embodiment, the vector is a non-viral integration-competent vector based on transposases, integrases or nucleases together with an appropriate template for transposition, integration and homologous recombination, respectively. Reference is made to Woodard and Calos, 2015, which is incorporated herein by reference, for an overview of suitable vector systems.

A vector according to the disclosure, preferably a lentiviral or other retroviral vector, may further comprise other elements such as long terminal repeats (LTRs), trans-activation response (TAR) elements, primer activation signals (PASs), primer binding sites (PBSs), packaging signals, Rev-responsive elements (RREs), constitutive transport elements (CTEs), RNA transport elements (RTEs), central termination sequences (CTSs), central polypurine tracts (cPPTs), polypurine tracts (PPTs), posttranscriptional regulatory elements of e.g., human hepatitis B virus (HVBPRE) or woodchuck hepatitis virus (WHVPRE), internal ribosome entry sites (IRESs) or 2A peptide(-like) sequences, and recognition and coding sequences for small drug-regulated repressor proteins like the tetracycline (Tc)-controlled hybrid protein rtTR-KRAB and the CymR repressor.

The LTRs contain repetitive sequences important for the transcription (e.g., TAR elements), reverse transcription (e.g., PASs) and host chromosomal DNA integration of retroviral genomes. Reverse transcription of retrovirus genomes in general further depends on PBSs and PPTs, while the reverse transcription of lentivirus and lentiviral vector genomes also requires CTSs and cPPTs. The CTSs and cPPTs also play a role in the nuclear import of reverse transcribed lentiviral genomes.

Packaging signal direct the incorporation of retrovirus genomes into viral particles.

The RREs are important for the efficient nucleocytoplasmic export of lentivirus and lentiviral vector-encoded unspliced and incompletely spliced RNAs including the lentiviral genome that is essential to generate viral progeny as retroviral packaging occurs in the cytoplasm. CTEs and RTEs have similar effects.

The expression of genes delivered by retroviral vectors can be inefficient. The posttranscriptional regulatory elements of Hepadnaviridae and the RREs, CTEs and RTEs of Retroviridae might help resolve this problem. Insertion of such elements in the untranslated regions (UTRs) of the coding sequences of a lentiviral or other retroviral vector may increase their expression level.

An internal ribosome entry site (IRES) is a nucleotide sequence that allows for 5′-end/cap-independent initiation of translation and thereby raises the possibility to express 2 proteins from a single messenger RNA (mRNA) molecule. IRESs are commonly located in the 5′ UTR of positive-stranded RNA viruses with uncapped genomes. Another means to express 2 proteins from a single mRNA molecule is by insertion of a 2A peptide(-like) sequence in between their coding sequence. 2A peptide(-like) sequences mediate self-processing of primary translation products by a process variously referred to as “ribosome skipping,” “stop-go” translation and “stop carry-on” translation. 2A peptide(-like) sequences are present in various groups of positive- and double-stranded RNA viruses including Picornaviridae, Iflaviridae, Tetraviridae, Dicistroviridae, Reoviridae and Totiviridae.

rtTR-KRAB binds to Tet operator (tetO) sequences in the absence, but not in the presence, of a Tc class antibiotic. Binding of rtTR-KRAB to tetO sequences in the vicinity of promoters inhibits transcription. After binding to a Tc class antibiotic, rtTtR-KRAB undergoes a conformational change strongly weakening its interaction with −tetO sequences thereby allowing transcriptional activation of a nearby promoter. CymR binds to the cumate operator site (CuO), which, when inserted immediately downstream of a promoter blocks transcription originating from this promoter. Addition of cumate, a small non-toxic molecule, relieves the repression.

As used herein, the term “promoter sequence” refers to a region of a nucleic acid sequence that is essential for the initiation of transcription of a nucleic acid sequence of interest. Transcription regulatory factors can bind to a promoter sequence.

In a preferred embodiment, the promoter is a cell type- or tissue-specific promoter and/or a promoter that is active in a differentiated form of the eukaryotic cell. Any promoter that is active in the differentiated form of the type of cell that is to be provided with the vector can be used. Hence, in a preferred embodiment, the promoter is a promoter that is active in the differentiated form of the eukaryotic cell. The activity of a promoter that is active in a differentiated form of the eukaryotic cell in the context of the disclosure is preferably not significantly less active and preferably not active in a less differentiated progenitor of the eukaryotic cell. Significantly less active is preferably a level of activity of the promoter that is at most 50% of the level, more preferably at most 30% of the level and more preferably at most 10% of the level in the differentiated cell. The level of activity is preferably measured by measuring the level of mRNA produced from the promoter in the untransformed cell and comparing the level in a differentiated form of the cell with the level in an undifferentiated form of the same cell, or a different but otherwise comparable untransformed cell.

As used herein, a “differentiated form” of a cell or a differentiated cell, e.g., of a cardiomyocyte, refers to a cell that is more specialized in its fate or function than at a previous stage in the cell's development. During development, a cell progresses from an uncommitted cell type, such as a PSC, typically via lineage-restricted precursor cells, to a specific differentiated cell type. The term includes both cells that are terminally differentiated and cells that are not (yet) terminally differentiated but are more specialized than at a previous stage in their development. A differentiated form of a cell is thus a cell with a mature phenotype specific for a particular tissue or organ system. A “proliferative form” of a cell refers to a cell undergoing mitosis. Typically, in a normal tissue proliferative forms of a cell are not yet terminally differentiated cells. In the methods of the disclosure, a proliferative phenotype is induced by expressing one or more immortalization genes in a cell that can be a differentiated cell isolated from a tissue of interest. In the methods of the disclosure, immortalization, and thus the establishment of a proliferative phenotype, is reversible. Following transcriptional repression of immortalization gene and/or protein expression, the cells again acquired a more differentiated phenotype. The use of a promoter for initiating expression of an immortalization gene, resulting in dedifferentiation and proliferation of a cell, that is active in a differentiated form of the cell is counterintuitive because it is to be expected that especially a cell type-specific promoter will become less active once the cell progresses into a more dedifferentiated and proliferative phenotype. In addition, once it is desired that immortalization is reversed and re-differentiation of the cell is induced a promoter that is active in a differentiated form of the cell would be intrinsically more active following cell differentiation than before, whereas in the methods of the disclosure transcription of the gene that is controlled by the promoter should be inhibited. However, the present inventors found that the use of a promoter that is active in a differentiated form of the eukaryotic cell actually leads to a better result, i.e., the advantages of the disclosure include the production of cells that very closely resemble the primary differentiated cells from which they were derived prior to immortalization, the synchronicity of the differentiation process and/or the high percentage of differentiated cells that are obtained.

A “tissue-specific promoter” as used herein refers to a promoter that initiates expression of the gene of interest essentially only in certain tissues. For example, a cardiac tissue-specific promoter initiates expression essentially only in cardiac tissue. A “cell type-specific promoter” as used herein refers to a promoter that initiates expression of the gene of interest essentially only in a specific cell type or only in a specific cell type in a particular tissue, although it may also initiate expression of the gene in another cell type in another tissue. For example, a cardiomyocyte-specific promoter initiates expression essentially only in cardiomyocytes or only in cardiomyocytes as the only cell type in cardiac tissue. For instance, the MHCK7 promoter (Salva et al., 2007) initiates expression both in cardiomyocytes and in skeletal muscle cells. But, since skeletal muscle cells are not present in cardiac tissue, such cells cannot be immortalized in a cardiac tissue sample and the MHCK7 is suitable for use in the disclosure. If the promoter is a tissue-specific promoter, it is preferred that the eukaryotic cell is a cell derived from the same tissue. Preferably, the tissue is cardiac tissue, more preferably, the eukaryotic cell is a cardiac cell, such as a cardiac fibroblast, a cardiomyocyte, an epicardial mesothelial cell or any other cardiac cell type like a nodal cell or a cardiac Purkinje cell, and the promoter is a cardiac tissue-specific promoter. A “cardiac tissue-specific promoter” refers to a promoter that initiates substantially higher levels of transcription in cardiac cells, such as cardiac fibroblasts, cardiomyocytes and epicardial mesothelial cells than in non-cardiac tissue cells. If the promoter is a cell type-specific promoter, it is preferred that the eukaryotic cell is a cell of the same type for which the promoter is specific. Preferably, the cell for which the promoter is specific and the eukaryotic cell are a cardiac cell, such as a cardiac fibroblast, a cardiomyocyte, an epicardial mesothelial cell or any other cardiac cell type like a nodal cell or a cardiac Purkinje cell. In a particularly preferred embodiment, the promoter is a cell type-specific promoter. Preferably, a promoter is cardiac cell type- or tissue-specific if the transcription levels induced by the promoter in cardiac cells are at least 2-fold, 3-fold, 4-fold, 5-fold, 6-fold, 7-fold, 8-fold, 9-fold, 10-fold, 50-fold, 100-fold, 500-fold or 1000-fold higher or more as compared to the transcription levels initiated by the promoter in other tissues.

In a particularly preferred embodiment, the promoter is a tissue-specific promoter, the eukaryotic cell is a primary cell derived from the same tissue and the promoter is active in a differentiated form of the cell. In another particularly preferred embodiment, the promoter is a cardiac cell-specific promoter, such as a cardiac fibroblast-specific promoter, a cardiomyocyte-specific promoter, an epicardial mesothelial cell-specific promoter, a nodal cell-specific promoter or a cardiac Purkinje cell-specific promoter, the eukaryotic cell is a primary cardiac cell of the same cell type and the promoter is active in a differentiated form of the cell.

In a further preferred embodiment, the promoter is a differentiated cardiac cell-specific promoter and the eukaryotic cell is a cardiac cell, preferably a primary cardiac cell. A particularly preferred promoter is the MHCK7 promoter if the eukaryotic cell is a cardiomyocyte. A further preferred promoter is the human eukaryotic elongation factor 1A1 (EEF1A1) gene promoter if the eukaryotic cell is a cardiac fibroblast, a preadipocyte or an epicardial mesothelial cell.

In one embodiment the promoter for the conditional immortalization of parenchymal cells is a cell type-specific promoter whose expression is relatively high and constant during phenotypic switching, i.e., is not much different between cells in the proliferation and differentiation stage. The RNA-Seq analyses has yielded several cardiomyocyte-specific genes whose expression changes little during phenotypic switching, including the cardiac tropin T (TNNT2) gene. In one embodiment the promoter of this gene is used to generate conditionally immortalized cardiomyocytes. Further provided is thus a vector as described herein comprising an immortalizing gene under transcriptional control (in operative linkage) with the TNNT2 gene promoter.

Further examples of promoters that are active in differentiated forms of cells include the ectoderm-specific Nes promoter, endoderm-specific Afp promoter, mesoderm-specific Mesp1 and T promoter, the epithelial Amy1c, Aqp5, Cftr, F5tg83, Klk1 and Krt18 promoter, the muscle-specific C5-12, CK5/6/7/8/9, Ckm, Des, dMCK, Hrc, Mb TMCK and Unc45b promoter, the cardiomyocyte-specific cardiac Actc1, Atp2a2, Cacna1c, Gja5 (atrial myocytes), Hcn4 (nodal cells), Irx4 (ventricular myocytes), Myh6/7, Myl2/3 (ventricular myocytes), Myl4/7 (atrial myocytes), Ncx1, Nppa (atrial myocytes), Sln (atrial myocytes) Tnni3 and Tnnt2 promoter, and, the cardiac progenitor cell- and cardiomyocyte-specific Nkx-2.5 promoter, the smooth muscle cell-specific Acgt2, Myh11, Mylk and Tagln promoter, the endothelial cell-specific Cdh5, Edn1, Flt1, Icam2, Kdr, Nos3, Sele, Smtn, Tie1/2, Serpine1 and Vwf promoter, the hepatocyte-specific Alb, Ambp, Apoa1/2/3/4/5, Apob, Apoc1/2/3/4, Apod, Apoe, Fabp1, Lcat, Otc, Pck1, Serpina1a/7 and Ttr promoter, the hematopoietic cell-specific Was promoter, the macrophage/microglial cell/Kupffer cell/osteoclast/histiocyte-specific CD68 promoter, the antigen-presenting cell-specific Clec7a, Dc-stamp, Fscn1 and Hla-Dra promoter, the pancreocyte-specific Amy2a1/2/3/4/5, Ins1/2 and Pdx1 promoter, the adipocyte-specific Adipoq, Fabp4 and Lep promoter, the podocyte-specific Npsh1/2 promoter, the keratinocyte-specific HPV LCR, EBV ED-L2 and Krt1/3/5/14 promoter, the pituitary gland cell-specific Cga, Gh1/2 and Prl promoter, the pneumocyte-specific Sftpb/c promoter, the renal tubular cell-specific Ggt1 and Sglt2 promoter, the neuron-specific Aldhl1l, Camk2a, Dbh, Dlx5/6, Eno2, Gad1, Gria1, Kcnc1, Nfh, Pdgfb, Penk, pUNISHER, Slc17a6/7/8, Stmn2, Syn1, Tac1, Th and Tuba1a promoter, the neuron and astrocyte-specific Slc1a2 promoter, the neuron- and tubular renal cell-specific Gls promoter, the glial cell-specific Gfap promoter, the oligodendrocyte-specific Mag and Mbp promoter, the photoreceptor cell-specific Crx, Grk1, Nrl, Opnl1/m/sw and Opn2 promoter as well as synthetic and chimeric cell type-specific promoters composed of (multiple) cellular and/or viral transcription regulatory elements including core promoters, proximal promoter elements, enhancers, silencers, insulators and locus control regions. The promoter is preferably not a (r)tTA-dependent transactivation promoter. The promoter that is operably linked to the immortalization gene is preferably not a promoter that has tetO sequence for (r)tTA-dependent transactivation. The vector preferably does not encode a tetracycline-regulated transactivator.

As used herein, the term “immortalization gene” refers to a gene encoding a protein that triggers cell division, inhibits (programmed) cell death, and/or promotes telomere synthesis. A vector according to the disclosure comprises at least one immortalization gene. Hence, more than one, such as two, immortalization genes may be present in the vector. In a preferred embodiment, a vector according to the disclosure comprises one or more immortalization genes, more preferably one. Any immortalization gene known in the art can be used in the vector according to the disclosure. Examples of suitable immortalization genes include genes encoding the polyomavirus T antigens, the adenovirus E1A and E1B gene products, the papillomavirus E6 and E7 proteins, the hepadnavirus X protein, the Epstein-Barr virus EBNAs, LMPs, BALF1 protein, BARF1 protein, BCRF1 protein and BART gene products, the Kaposi's sarcoma virus-associated herpesvirus K1 protein, K2 protein, K15 protein, ORF72 product, ORF74 product, LANA and kaposin, the herpesvirus saimiri STP, TIP, ORF13, ORF72, ORF73 and ORF74 products, the human T-cell leukemia virus type 1 Tax and HBZ proteins, the hepatitis C virus core, NS3 and NS5A proteins, dominant-negative versions of Cip/Kip, Ink4, p53 and Rb family members, constitutively active versions of cyclins, CDKs and Mdm2, Polycomb group proteins (e.g., Bmi1, Cbx7 and Cbx8), anti-apoptotic/autophagic/necroptotic/necrotic proteins (e.g., apollon, cIAP1, cIAP2, ILP2, livin/MLIAP, NIAP, survivin, A1, Bcl-2, Bcl2-L-10, Bcl-B, Bcl-W, Bcl-XL, Bfl-1, BOO/DIVA, Mcl-1, NR-13, vBcl-2, vIAP, C-FLIP, V-FLIP, adenovirus E3-6.7k protein, RIDα, RIDβ, E3-14.7k protein, African swine fever virus A179L [5-HL] protein, baculovirus p35, cytomegalovirus J32.7, pUL37x1 [vMIA], UL36 [vICA], vIRA, Epstein-Barr virus BHRF1 protein, fowlpox virus FPV039 protein, γ herpesvirus 68 M8 [vMAP] protein, M11 protein, hepatitis C virus E2, NS2, NS5A, herpes simplex virus ICP34.5 protein, US3 protein, US5 protein, herpesvirus pan and papio hpnBHRF 1, herpesvirus saimiri ORF 16 product, influenzavirus M2 Kaposi's sarcoma-associated herpesvirus, K7 protein, K13 protein, KSBcl-2, myxomavirus M11L, parapoxvirus ORFV125, vaccinia virus CrmaA/SPI-2, F1L, N1L, SPI-1), retroviral oncoproteins (e.g., v-Abl, v-Akt, v-Crk, v-Cyclin, v-ErbA, v-ErbB, v-Fos, v-Ha-ras, v-Jun, v-Ki-ras, v-Mos, v-Myc, v-P3K, v-Sis and v-Src) and telomerase reverse transcriptase as well as carcinogenic mutants of cellular oncogenes and tumor suppressor genes. In a preferred embodiment the immortalization gene encodes SV40 LT or a variant thereof, preferably a temperature-sensitive variant, preferably temperature-sensitive mutant tsA58.

As used herein, the term “inducible regulator” refers to a regulator activity that can be induced by an external factor, such as a ligand that can be added to or removed from a composition to induce or repress the activity of the regulator. Suitable regulators for use in the disclosure are described herein elsewhere. Further reference is made to Weber and Fussenegger 2006 that is incorporated by reference herein, for a description of inducible systems and regulators that can be used in accordance with the disclosure.

As used herein, the term “operably linked” refers to the association of nucleic acid sequences on the vector so that the function of one is affected by the other. An inducible regulator operably linked to the promoter sequence means that the activity of the promoter sequence is regulated by the inducible regulator. At least one immortalization gene operably linked to the promoter sequence means that the promoter initiates transcription of the at least one immortalization gene.

The inducible regulator can either be a repressor or an activator, i.e., repress or activate the activity of the promoter. In a preferred embodiment, the inducible regulator is a repressor.

As used herein, the term “response element” refers to a nucleic acid or protein sequence, which, when bound by a ligand or ligand-inducible regulator, causes a decrease in transgene expression by affecting mRNA biosynthesis, RNA transport, mRNA stability, protein biosynthesis, protein stability, protein transport or protein activity. In a particular embodiment, when an inducible promoter system as described below, such as a Tet system, is used, a response element refers to a sequence that effects an increase or decrease in gene expression when bound by a nucleic acid-binding (e.g., DNA-binding) protein. The “response element” is preferably a nucleic acid sequence.

Inducible promoter systems are well known in the art. Examples of inducible promoter systems that can be used in a vector according to the disclosure include promoter systems regulated by antibiotics (e.g., tetracyclines, streptogramins, rapamycin, rapalogs, macrolides), cytokines (e.g., interferons, interleukins, tumor necrosis factor), steroid hormones (e.g., dexamethasone, ecdysone, [4-hydroxy]tamoxifen), acetaldehyde, ethanol, forskolin, glucose, insulin, metals, mifepristone, nitric oxide, genotoxic, metabolic or oxidative stress, hypoxia, light or temperature.

A preferred inducible promoter system is a Tet-inducible promoter system. The Tet expression system can be a Tet-On or a Tet-Off system. Briefly, in the Tet-On system, transcription is active in the presence of Tc or a Tc derivative like doxycycline (Dox). In the Tet-Off system, transcription is inactive in the presence of Tc or Dox.

In the Tet-Off expression system, the Tet repressor DNA binding protein (TetR) from the Tc resistance operon of Escherichia coli transposon Tn10 is fused to the strong transactivating domain of VP 16 from Herpes simplex virus yielding a Tc-controlled transactivator protein (tTA). This fusion protein regulates expression of the target gene that is under transcriptional control of a Tc-response element (TRE), as part of the promoter sequence in a vector according to the present disclosure if a Tet-Off system is used. A TRE typically consists of multiple repeats of a 19-nucleotide tetO sequence (TCCCTATCAGTGATAGAGA) separated by spacer sequences, and is recognized by TetR. In the absence of Tc or Dox, tTA binds to the TRE and activates transcription of the target gene. In the presence of Tc or Dox, tTA can no longer bind to the TRE, and target gene expression is lost. In an alternative design of the Tet-Off expression system, a mutant version of TetR (see below) is fused to a Kruppel-associated box or KRAB domain with long-range epigenetic silencing activity yielding a reverse Tc-controlled repressor protein (rtTR, also referred to as tTS). Binding of rtTR to a TRE located in the vicinity of a promoter inhibits its activity. In the absence of Tc or Dox, rtTR cannot bind to the TRE and repress the nearby promoter resulting in transcription of the target gene. In the presence of Tc or Dox, rtRT binds to the TRE and represses target gene expression.

The Tet-On system is based on a reverse Tc-controlled transactivator designated rtTA. Like tTA, rtTA is a fusion protein comprised of the TetR and the VP16 transactivation domain. However, due to a four amino acid change in the TetR DNA binding moiety the binding characteristics of rtTA are altered. Contrary to tTA, rtTA recognizes the tetO sequences in the TRE of the target transgene in the presence of Tc or Dox. Thus, in the Tet-On system, transcription of the TRE-regulated target gene is stimulated by rtTA only in the presence of Tc or Dox. In an alternative design of the Tet-On expression system, TetR is fused to a KRAB domain yielding a Tc-controlled repressor protein (tTR). Binding of tTR to a TRE located in the vicinity of a promoter inhibits its activity. In the absence of Tc or Dox, tTR binds to the TRE and represses target gene expression. In the presence of Tc or Dox, tTR can no longer bind to the TRE, and target gene expression is induced. Such Tet-On system is a preferred system for use in accordance with the disclosure.

Another example of a suitable inducible promoter system is the Lac repressor (LacR) system from Escherichia coli. The LacR system functions by regulating transcription of a target gene with a promoter that contains lac operator (lacO) sequences that LacR can bind to. Expression of the target gene is induced by isopropyl-β-D-thiogalactopyranoside (IPTG). After binding to IPTG, LacR undergoes a conformational change strongly weakening its interaction with lacO sequences thereby allowing transcriptional activation of the promoter.

In a preferred embodiment, the inducible regulator is a Tc-responsive transcriptional repressor, and the response element is a TRE.

Another example of a suitable inducible and reversible expression system for use in accordance with the disclosure is a ligand-inducible degron. A suitable, but non limiting, example is an auxin-inducible degron. By coupling of the protein-coding domain of the immortalization gene to a ligand-inducible degron, its stability can be regulated by addition or retention of the ligand. Depending on the selected system, the presence of the ligand can either stabilize or destabilize the immortalization factor. Further, in ligand-inducible degron systems the response element is the degron itself and increase or decrease of expression of the immortalization gene results from binding of the ligand to the degron. Suitable ligand-inducible degrons that can be used in accordance with the disclosure are, for instance, described in Bonger et al. (2011), Holland et al. (2012) and Navarro et al. (2016), which are incorporated herein by reference. Expression of the immortalization factor fused to a degron can be regulated using any one the promoters described herein above.

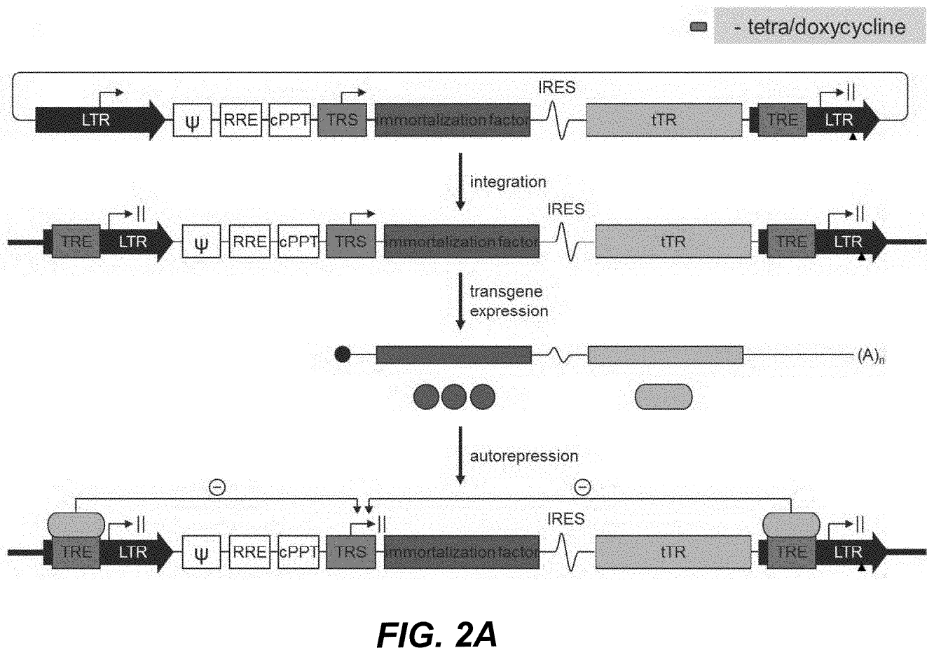

A particularly preferred vector is a vector as shown in FIGS. 2A and 2B.

In a further preferred example, the vector system used for the reversible immortalization in accordance with the disclosure is based on an inducible (i.e., ligand-controlled) HIV type 1 (HIV1) vector system developed by Szulc and co-workers. The starting construct (pLVET; https://www.addgene.org/11644/) was genetically modified to allow rapid insertion, in between the HIV1 CTS and the encephalomyocarditis virus IRES, of transcriptional regulatory sequences (TRSs; e.g., promoter sequences) followed by the coding sequences of immortalization genes (FIG. 2A). In the absence of ligand (e.g., Tc or Dox in FIG. 2A) immortalization gene expression is blocked by autorepression (FIG. 2A). In the presence of ligand (e.g., Tc or Dox), tTR undergoes a conformational change that no longer allows it to bind to its target sequence and to inhibit immortalization gene expression (FIG. 2B). Accordingly, in culture medium with ligand, cells undergo proliferation due to the presence of a high (enough) amount of immortalization factor while in culture medium without ligand the cells stop dividing and can acquire their original differentiated phenotype due to the repression of immortalization gene expression.

As is shown in the Examples, the ligand-dependent (e.g., Tc- or Dox-dependent) expression of the immortalization gene(s) typically changes the properties of (differentiated) cells causing them to adopt a more undifferentiated phenotype. However, surprisingly, the (immortalized) cells (largely) maintain their epigenetic memory allowing them to (spontaneously) re-differentiate following shutdown of immortalization gene expression (i.e., Tc or Dox removal). For example, prior to immortalization, atrial cardiomyocytes showed well-developed sarcomeres (i.e., contractile elements), were excitable (i.e., could generate a cardiac action potential), did contract and rapidly conducted cardiac action potentials (at ±20 cm/sec). All these properties were lost in the proliferative state (i.e., in the presence of Tc or Dox) but reacquired after ligand removal without the need to use complex cocktails of specific differentiation factors.

Molecular cloning procedures using the starting construct (pLVET-tTR-KRAB) described by Szulc et al. (2006) were not straightforward due to: (1) the lack of convenient restriction enzyme/recombinase recognition sequences at strategic positions in this construct and (2) the specific arrangement of certain genetic elements. Therefore, a genetically modified version of pLVET-tTR-KRAB was generated by: (1) removing the promoter driving transgene expression (i.e., the human EEF1A1 gene promoter), (2) insertion of a multiple cloning site downstream of the HIV1 CTS and (3) removing the coding sequence for the enhanced green fluorescent protein (eGFP). Through these genetic modifications, insertion into the plasmid of all kinds of transgenes (with different promoters driving the expression of the desired RNA/protein-coding sequences) has become very easy.

Further provided herein is a genetically modified eukaryotic cell provided with a vector according to the disclosure. Hence, the cell comprises at least one immortalization gene operably linked to a promoter sequence, preferably a cell type- or tissue-specific promoter and/or a promoter that is active in a differentiated form of the cell that is under the control of an inducible regulator.

As used herein a “genetically modified eukaryotic cell” refers to a eukaryotic cell that contains a nucleotide sequence and/or protein or is transformed or genetically modified with a nucleotide sequence that does not naturally occur in the cell, i.e., a eukaryotic cell into which an exogenous nucleotide sequence has been introduced. In the cell of the disclosure, this nucleotide sequence is at least the at least one immortalization gene.

It is preferred that the cell is transiently or stably transfected or transduced with one or more nucleic acid molecules. In a preferred embodiment, the cell according to the present disclosure comprises the inducible promoter sequence, the regulator sequence operably linked with the promoter sequence and the at least one immortalization gene operably linked with the inducible promoter sequence in its genome. This can, for instance, be achieved if the cell is transduced with a viral vector according to the present disclosure and the indicated nucleotide sequences have been introduced in the cell's chromosomal DNA.

In another embodiment, the eukaryotic cell is provided with a vector according to the disclosure using persisting episomes. Reference is made to Lufino et al. (2008), which is incorporated by reference herein, for suitable methods. In yet another embodiment, the eukaryotic cell is provided with a vector according to the disclosure using non-cytopathogenic RNA virus replicons. Reference is made to Schott et al. (2016), which is incorporated by reference herein, for suitable methods. By using such approaches, the immortalization gene is not incorporated into the cell's genome.

The genetically modified eukaryotic cell is preferably a primary cell or progeny thereof. As used herein a “primary cell” refers to a cell that is cultured directly from a subject. As used herein a “progeny” of a primary cell refers to a cell of any subsequent generation that has been derived from a primary cell through cell division.

The genetically modified eukaryotic cell is preferably a human cell, a rat cell, a mouse cell, a canine cell or a porcine cell. The cell can be a heart cell, a blood vessel cell, a cell of the gastrointestinal, respiratory or urogenital tract (e.g., a gut cell, a kidney cell, a liver cell, a lung cell, a pancreatic cell, a stomach cell and a prostate cell), a brain cell, a nerve cell, a skin cell, a glandular cell, a skeletal or smooth muscle cell, a bone cell, a cartilage cell, a connective tissue cell, a fat cell, a hematopoietic cell, a sensory organ cell or any other cell type of the body. The cell can be a differentiated cell of the specified type. The differentiated cell can be isolated from embryonic, fetal, neonatal, juvenile or adult tissue.

In one aspect a differentiated cell is provided with a vector of the disclosure and triggered to dedifferentiate at least in part, such that the cell proliferates or keeps proliferating. The dedifferentiation that allows the multiplication of the cells is not to be confused with the pluripotency-inducing transformation underlying the generation of induced PSC.

The genetically modified eukaryotic cell is preferably a cardiac cell. It is preferably an atrial cardiac cell, a cardiomyocyte, a cardiac fibroblast, an epicardial mesothelial cell, a cardiac nodal cell or a cardiac Purkinje cell. The cell is preferably a primary cardiac cell, preferably a human, rat, mouse, canine or porcine cardiac cell.

Also provided is a cell line comprising a plurality of cells according to the disclosure. A “cell line” as used herein refers to a propagated culture of cells. The cell line preferably comprises a plurality of primary cells or progeny thereof. As used herein a “plurality of cells” refers to at least 2 cells. However, a cell line preferably comprises multiple cells. Hence, a plurality preferably refers to at least 3, 4, 5, 10, 100, 500, 1000 or more cells.

The primary cell or progeny thereof is preferably a mammalian cell, such as a mouse, rat or human cell or an avian cell, more preferably a mammalian cell. In a particularly preferred embodiment the cell is a human cell. The cell can be any type of cell derived from a mammal or bird. Examples are a cardiac cell, cardiomyocyte, cardiac or other fibroblast, cardiac or other myofibroblast, cardiac valve or other interstitial cell, cardiac Purkinje cell, vascular cell, smooth muscle cell, pericyte, endothelial cell, epicardial mesothelial or interstitial cell, pericardial mesothelial or interstitial cell, pleural mesothelial or interstitial cell, peritoneal mesothelial or interstitial cell, basal cell, apical cell, brain cell, neuron, glial cell, astrocyte, satellite cell, oligodendrocyte, Schwann cell, microglial cell, ependymal cell, liver/hepatic cell, hepatocyte, hepatic stellate/Ito cell, Kupffer cell, ductal cell, blood/hematopoietic cell, bone marrow cell, blast cell, leukocyte, granulocyte, macrophage, monocyte, megakaryocyte, lymphocyte, reticulocyte, natural killer cell, mast cell/mastocyte, thymocyte, kidney/renal cell, tubular cell, podocyte, mesangial cell, mesenchymal cell, cementocyte, chondrocyte, dental pulp cell, vitreous cell/hyalocyte, odontocyte, osteocyte, synoviocyte, tenocyte, pancreatic cell, gland(ular) cell, serous cell, mucous cell, acinar cell, α cell, β cell, γ/PP cell, δ cell, ε cell, skeletal muscle cell, adipose tissue cell, fat cell/adipocyte, skin/epidermal cell, keratinocyte, melanocyte, Langerhans/dendritic cell, epithelial cell, myoepithelial cell, thyroid cell, follicular cell, lymph node cell, eye/ocular cell, retinal cell, photoreceptor cell, cone cell, rod cell, ear/acoustic cell, hair cell, cell of Boettcher, cell of Claudius, cell of Deiters, cell of Hensen, nose/olfactory cell, urether cell, bladder cell, urethra cell, spleen cell/splenocyte, intestinal cell, pharyngeal cell, esophageal cell, stomach cell, jejunal cell, duodenal cell, small intestine/ileal cell, large intestine/colonal cell, rectal cell, anal cell, enterocyte, Paneth cell, tuft cell, lung cell, pneumocyte, ciliated cell, Clara cell, goblet cell, neuroendocrine cell, oncocyte, germ cell, gonocyte, nurse cell, ovarian cell, uterus cell, cervical cell, vaginal cell, seminal gland cell, prostate cell, epididymal cell, testicular cell, Leydig cell and Sertoli cell. In a preferred embodiment, a cell according to the present disclosure, preferably a primary cell or progeny thereof, is a cardiac cell, such as a cardiac fibroblast, a cardiomyocyte or an epicardial mesothelial cell. It is further preferred that the cell is a cardiac cell and the promoter is a differentiated cardiac cell-specific promoter. A “differentiated cardiac cell-specific promoter” refers to a promoter that is specific for a differentiated form of a cardiac cell.

In one embodiment, the cell or cell line according to the disclosure is a proliferative cell or cell line. The cell or cell line is, for instance, cultured in the presence of the ligand of the inducible regulator operably linked to the promoter, which ligand induces transcription of the immortalization gene in the cell or in the cells of the cell line. Alternative the cell or cell line is cultured in the absence of a ligand of the inducible regulator operably linked to the promoter, which ligand inhibits transcription of the immortalization gene in the cell or in the cells of the cell line. In yet another embodiment, the cell or cell line according to the disclosure is a differentiated or redifferentiated cell or cell line. This is, for instance, achieved by removing a ligand of the inducible regulator operably linked to the promoter. This ligand induces transcription of the immortalization gene in the cell or in the cells of the cell line from a cell culture comprising the cell or cell line, or by adding a ligand of the inducible regulator operably linked to the promoter, which ligand inhibits transcription of the immortalization gene in the cell or in the cells of the cell line to a cell culture comprising the cell or cell line. The term “redifferentiated” in this context refers to the differentiation of a cell or cells originally derived from a differentiated cell, e.g., a primary cell or a progeny cell thereof that has been immortalized with a method of the disclosure.

Also provided is a kit of parts comprising a cell or cell line according to the disclosure and an agent capable of activating or repressing the inducible promoter. The specific agent present in the kit of parts depends on the inducible system that is used in the vector provided to the cell or cell line. Inducible systems and the respective agents that activate or inhibit the inducible promoter thereby initiating or inhibiting transcription of the target nucleic acid sequence(s), i.e., the one or more immortalization genes in the disclosure, are known in the arts and examples have been described above. As described above, in a preferred embodiment, a Tc-inducible promoter system that can be a Tet-On system or a Tet-Off system is used in the vector, cell and methods of the disclosure. Hence, in a preferred embodiment, the cell has been provided with a vector wherein the inducible regulator is a Tc-responsive transcriptional repressor or activator, and the response element is a TRE, and the agent capable of activating or repressing the inducible promoter is a Tc class antibiotic. A used herein the term “Tc class antibiotic” refers to Tc or a compound that has the same effect of Tc in a Tet expression system, such as Dox. Preferred Tc class antibiotics are Tc and Dox.

The kit of parts may further comprise one or more components facilitating proliferation, survival and differentiation of the cell or cell line according to the disclosure. The specific components may depend on the type of cell or cell line, e.g., whether these are cardiac cells, neural cells, vascular cells, dermal cells, intestinal cells, hepatic cells, renal cells, and so forth. In a preferred embodiment, the one or more components are selected from the group consisting of natural or synthetic extracellular matrix components (e.g., collagen, fibronectin, gelatin, laminin, proteoglycans, vitronectin and derivatives thereof), anti-oxidants and vitamins (e.g., ascorbic acid [2-phosphate]/vitamin C, γ-tocopherol/vitamin E, glutathione, N-acetylcysteine, Qter, quercetin, resveratrol, retinoic acid/vitamin A, vitamin B1-7, B9 and B12, catalase, superoxide dismutase and glutathione peroxidase), trace elements (e.g., iron, selenium and zinc), lipids (e.g., cholesterol, phospholipids, sphingolipids and [un]saturated fatty acids), reducing agents (e.g., 2-mercaptoethanol, dithiothreitol, dithioerythritol and tris-[2-carboxyethyl]-phosphine hydrochloride), histone deacetylase inhibitors (butyric acid, [suberoylanilide]hydroxamic acid, trichostatin A and valproic acid), DNA methyltransferase inhibitors (e.g., 5-azacytidine, decitabine, hydralazine and zebularine), caspase inhibitors (emricasan, Q-VD-OPh and Z-VAD-FMK), natural and synthetic hormones (e.g., angiotensin, dexamethasone, endothelin, growth hormone, insulin, phenylephrine, prostaglandins and [para]thyroid hormone), agonists and antagonists of the Akt, AMPK, apoptosis, ErbB/HER, estrogen, HGF/c-Met, Hippo/YAP, insulin, JAK/STAT, MAPK/ERK, mTOR, NF-κB, Notch, p53, SCF/c-Kit, TGF-β/BMP, TLR, VEGF and Wnt signaling pathways (e.g., activin, adipokine, bone morphogenetic protein, chemokine, colony-stimulating factor, cytokine, epidermal growth factor, fibroblast growth factor, insulin-like growth factor, interferon, interleukin, myokine, nerve growth factor, neuregulin, platelet-derived growth factor, tumor necrosis factor, vascular endothelial growth factor, wnt, Dkk and sFRP family members, CHIR99021, dorsomorphin, famotidine, IWP3, IWR1, noggin, SB208, SB203580, SB431542 and XAV939), L-carnitine, (phospho)creatine, polyamines and (phosphoenol)pyruvate. Therefore, in a preferred embodiment the kit of parts comprises a human cardiac cell or cell line and the kit further comprises one or more components selected from the group consisting of natural or synthetic extracellular matrix components (e.g., collagen, fibronectin, gelatin, laminin, proteoglycans, vitronectin and derivatives thereof), anti-oxidants and vitamins (e.g., ascorbic acid [2-phosphate]/vitamin C, γ-tocopherol/vitamin E, glutathione, N-acetylcysteine, Qter, quercetin, resveratrol, retinoic acid/vitamin A, vitamin B1-7, B9 and B12, catalase, superoxide dismutase and glutathione peroxidase), trace elements (e.g., iron, selenium and zinc), lipids (e.g., cholesterol, phospholipids, sphingolipids and [un]saturated fatty acids), reducing agents (e.g., 2-mercaptoethanol, dithiothreitol and dithioerythritol), histone deacetylase inhibitors (butyric acid, [suberoylanilide] hydroxamic acid, trichostatin A and valproic acid), DNA methyltransferase inhibitors (e.g., 5-azacytidine, decitabine, hydralazine and zebularine), caspase inhibitors (emricasan, Q-VD-OPh and Z-VAD-FMK), natural and synthetic hormones (e.g., angiotensin, dexamethasone, endothelin, growth hormone, insulin, phenylephrine, prostaglandins and [para]thyroid hormone), agonists and antagonists of the Akt, AMPK, apoptosis, ErbB/HER, estrogen, HGF/c-Met, Hippo/YAP, insulin, JAK/STAT, MAPK/ERK, mTOR, NF-κB, Notch, p53, SCF/c-Kit, TGF-3/BMP, TLR, VEGF and Wnt signaling pathways (e.g., activin, adipokine, bone morphogenetic protein, chemokine, colony-stimulating factor, cytokine, epidermal growth factor, fibroblast growth factor, insulin-like growth factor, interferon, interleukin, myokine, platelet-derived growth factor, tumor necrosis factor, vascular endothelial growth factor, wnt, Dkk and sFRP family members, CHIR99021, dorsomorphin, famotidine, IWP3, IWR1, noggin, SB208, SB203580, SB431542 and XAV939), L-carnitine, creatinine, polyamines, pyruvate and combinations thereof.

Also provided is a method for generating one or more conditionally immortalized cells, the method comprising introducing one or more vectors according to the disclosure into one or more eukaryotic cells resulting in one or more genetically modified eukaryotic cells. “Provided with a vector” as used herein preferably refers to transfection or transduction of the one or more primary cells with one or more vectors. In a preferred embodiment the vector according to the disclosure is a viral vector, more preferably a lentiviral or other retroviral vector, and the one or more primary cells are transduced with the vector. Preferred vectors used in the methods of the disclosure are preferred vectors described herein above. Preferably the promoter is a cell type- or tissue-specific promoter and/or a promoter that is active in a differentiated form of the cell, more preferably a cell type-specific promoter. It is further preferred that at least one immortalization gene encodes SV40 LT or a variant thereof, preferably a temperature-sensitive variant, preferably temperature-sensitive mutant tsA58. The eukaryotic cell that is provided with the vector is preferably a primary cell or progeny thereof, preferably from a mammal or a bird. It is further preferred that the cell, preferably a primary cell or progeny thereof, is a cardiac cell, such as a cardiomyocyte, cardiac fibroblast or pericardial mesothelial cell. In a particularly, preferred embodiment, the cell is a cardiac cell and the promoter is a differentiated cardiac cell-specific promoter, more preferably the chimeric striated muscle-specific MHCK7 promoter.

Once the conditionally immortalized cell or cells have been obtained, immortalization and/or proliferation of the cell or cells can be obtained by inducing expression of the one or more immortalization genes. Hence, also provided is a method of the disclosure further comprising culturing the one or more conditionally immortalized cells under conditions whereby transcription of the one or more immortalization genes is induced. In one embodiment, the method further comprises culturing the primary cells provided with the one or more vectors in the absence of an agent that inhibits activity of the promoter. For instance, when using a Tet-Off system, a method of the disclosure further comprises culturing primary cells provided with the one or more vectors in the absence of a Tc class antibiotic thereby activating the promoter and allowing transcription of the at least one immortalization gene. In another embodiment, the method further comprises culturing the primary cells provided with the one or more vectors in the presence of an agent that induces activity of the promoter resulting in transcription of the at least one immortalization gene. For instance, when using a Tet-On system, a method of the disclosure further comprises culturing primary cells provided with the one or more vectors in the presence of a Tc class antibiotic that induces release of the inducible repressor from the response element thereby activating the promoter and allowing transcription of the at least one immortalization gene.

The methods of the disclosure may further comprise stimulating differentiation of the conditionally immortalized cell or cells. As shown in the Examples, the use of a vector as described herein for conditionally immortalization of cells allows, following shutdown of the immortalization, differentiation of the cells to cells that very closely resembled the primary cells from which they were derived without the need for differentiation factors. Hence, a method according to the disclosure may further comprise culturing the one or more conditionally immortalized cells under conditions whereby transcription of the one or more immortalization genes is inhibited. In one embodiment, the method further comprises culturing the primary cells provided with the one or more vectors in the presence of an agent that inhibits activity of the promoter. For instance, when using a Tet-Off system, a method of the disclosure further comprises culturing primary cells provided with the one or more vectors in the presence of a Tc class antibiotic thereby inhibiting the activity of the promoter and the transcription of the at least one immortalization gene. In another embodiment, the method further comprises culturing the primary cells provided with the one or more vectors in the absence of an agent that induces activity of the promoter. For instance, when using a Tet-On system, a method of the disclosure further comprises culturing primary cells provided with the one or more vectors in the absence of a Tc class antibiotic thereby inhibiting the activity of the promoter and the transcription of the at least one immortalization gene.

Inducing differentiation, proliferation and/or survival by inhibition of transcription of the immortalization gene may be accelerated or improved by the addition of one or more components that facilitate such differentiation, proliferation and/or survival of the cell or cell line according to the disclosure. The specific components may depend on the type of cell or cell line, e.g., whether these are cardiac cells, neural cells, vascular cells, dermal cells, intestinal cells, hepatic cells, renal cells, and so forth. Hence, in one embodiment, a method according to the disclosure further comprises culturing the one or more conditionally immortalized cells under conditions whereby transcription of the one or more immortalization genes is inhibited and in the presence of one or more components facilitating survival and/or differentiation of the cells. Stimulation of proliferation is not desired following shut down of immortalization gene expression. In the proliferative state it may be attractive to add components that promote cell proliferation and survival while during cell differentiation it may be useful to add components that enhance cell differentiation and survival.

The one or more components are preferably selected from the group consisting of natural or synthetic extracellular matrix components (e.g., collagen, fibronectin, gelatin, laminin, proteoglycans, vitronectin and derivatives thereof), anti-oxidants and vitamins (e.g., ascorbic acid [2-phosphate]/vitamin C, γ-tocopherol/vitamin E, glutathione, N-acetylcysteine, Qter, quercetin, resveratrol, retinoic acid/vitamin A, vitamin B₁₋₇, B₉ and B₁₂, catalase, superoxide dismutase and glutathione peroxidase), trace elements (e.g., iron, selenium and zinc), lipids (e.g., cholesterol, phospholipids, sphingolipids and [un]saturated fatty acids), reducing agents (e.g., 2-mercaptoethanol, dithiothreitol, dithioerythritol and tris-[2-carboxyethyl]-phosphine hydrochloride), histone deacetylase inhibitors (butyric acid, [suberoylanilide] hydroxamic acid, trichostatin A and valproic acid), DNA methyltransferase inhibitors (e.g., 5-azacytidine, decitabine, hydralazine and zebularine), caspase inhibitors (emricasan, Q-VD-OPh and Z-VAD-FMK), natural and synthetic hormones (e.g., angiotensin, dexamethasone, endothelin, growth hormone, insulin, phenylephrine, prostaglandins and [para]thyroid hormone), agonists and antagonists of the Akt, AMPK, apoptosis, ErbB/HER, estrogen, HGF/c-Met, Hippo/YAP, insulin, JAK/STAT, MAPK/ERK, mTOR, NF-κB, Notch, p53, SCF/c-Kit, TGF-3/BMP, TLR, VEGF and Wnt signaling pathways (e.g., activin, adipokine, bone morphogenetic protein, chemokine, colony-stimulating factor, cytokine, epidermal growth factor, fibroblast growth factor, insulin-like growth factor, interferon, interleukin, myokine, nerve growth factor, neuregulin, platelet-derived growth factor, tumor necrosis factor, vascular endothelial growth factor, wnt, Dkk and sFRP family members, CHIR99021, dorsomorphin, famotidine, IWP3, IWR1, noggin, SB208, SB203580, SB431542 and XAV939), L-camitine, (phospho)creatine, polyamines and (phosphoenol)pyruvate. Therefore, in a preferred embodiment the kit of parts comprises a human cardiac cell or cell line and the kit further comprises one or more components selected from the group consisting of natural or synthetic extracellular matrix components (e.g., collagen, fibronectin, gelatin, laminin, proteoglycans, vitronectin and derivatives thereof), anti-oxidants and vitamins (e.g., ascorbic acid [2-phosphate]/vitamin C, γ-tocopherol/vitamin E, glutathione, N-acetylcysteine, Qter, quercetin, resveratrol, retinoic acid/vitamin A, vitamin B₁₋₇, B₉ and B₁₂, catalase, superoxide dismutase and glutathione peroxidase), trace elements (e.g., iron, selenium and zinc), lipids (e.g., cholesterol, phospholipids, sphingolipids and [un]saturated fatty acids), reducing agents (e.g., 2-mercaptoethanol, dithiothreitol and dithioerythritol), histone deacetylase inhibitors (butyric acid, [suberoylanilide] hydroxamic acid, trichostatin A and valproic acid), DNA methyltransferase inhibitors (e.g., 5-azacytidine, decitabine, hydralazine and zebularine), caspase inhibitors (emricasan, Q-VD-OPh and Z-VAD-FMK), natural and synthetic hormones (e.g., angiotensin, dexamethasone, endothelin, growth hormone, insulin, phenylephrine, prostaglandins and [para]thyroid hormone), agonists and antagonists of the Akt, AMPK, apoptosis, ErbB/HER, estrogen, HGF/c-Met, Hippo/YAP, insulin, JAK/STAT, MAPK/ERK, mTOR, NF-κB, Notch, p53, SCF/c-Kit, TGF-3/BMP, TLR, VEGF and Wnt signaling pathways (e.g., activin, adipokine, bone morphogenetic protein, chemokine, colony-stimulating factor, cytokine, epidermal growth factor, fibroblast growth factor, insulin-like growth factor, interferon, interleukin, myokine, platelet-derived growth factor, tumor necrosis factor, vascular endothelial growth factor, wnt, Dkk and sFRP family members, CHIR99021, dorsomorphin, famotidine, IWP3, IWR1, noggin, SB208, SB203580, SB431542 and XAV939), L-carnitine, creatinine, polyamines, pyruvate or combinations thereof.

Conditionally immortalized cells using the disclosure have a wide variety of application. They are, for instance, of great interest as model systems for, optionally high-throughput, screening of chemical libraries and evaluation of experimental drugs. Also, experiments with cells reversibly immortalized using the disclosure may provide an excellent alternative to the use of animals in (bio)pharmaceutical research, which is meeting with increasing public resistance and restrictive legislation. Cells and cell lines according to the disclosure or prepared in accordance with the disclosure are further suitable as disease model and/or for target identification, e.g., in disease models, for fundamental research, cell therapy and/or tissue engineering.

Features may be described herein as part of the same or separate aspects or embodiments of the disclosure for the purpose of clarity and a concise description. It will be appreciated by the skilled person that the scope of the disclosure may include embodiments having combinations of all or some of the features described herein as part of the same or separate embodiments.

### Examples

### Materials and Methods

### Construction of Plasmids

DNA constructions were carried out with enzymes from Fermentas (Thermo Fisher Scientific, Breda, The Netherlands) or from New England Biolabs (Bioke, Leiden, The Netherlands) and with oligodeoxyribonucleotides from Eurofins MWG Operon (Ebersberg, Germany) using established procedures or following the instructions provided with specific reagents.

To generate a thermosensitive version of the oncogenic SV40 LT antigen (mutant tsA58 also known as tsA438A-V; Loeber et al., 1989), plasmid pAT153.SV40ori(−) (Dinsart et al., 1984) was incubated with PvuII and the 4.3-kb digestion product was recircularized with bacteriophage T4 DNA ligase to generate pAT153.SV40ori(−).dPvuII. pAT153.SV40ori(−).dPvuII was subsequently cut with HindIII and PvuII and the large digestion product of 4.3 kb was combined with the hybridization product of oligodeoxyribonucleotides 5′ CTGTGCTTCTAGAATTATGTGGGGGGAA 3′ (SEQ ID NO:2) and 5′ AGCTTTCCCCCCACATAATTCTAGAAGCACAG 3′ (the mutant codon and its complement are underlined (SEQ ID NO:3)) producing pAT153.SV40ori(−).dPvuII.LT-tsA58. Next, pAT153.SV40ori(−)dPvuII.LT-tsA58 was linearized with PvuII and the 4.3-kb digestion product was ligated to the 2.0-kb PvuII fragment of pAT153.SV40ori(−) to generate pAT153.SV40ori(−).LT-tsA58. pAT153.SV40ori(−)LT-tsA58 was used as template in a PCR with VELOCITY DNA polymerase (GC Biotech, Alphen aan den Rijn, The Netherlands) and primers 5′ AGGTTTAAACTACGGGATCCGTGCACCATGGATAAAGTTTTAAACAGAGAGGA 3′ (SEQ ID NO:4) and 5′ CCGAATTCTTTATGTTTCAGGTTCAGGG 3′ (the initiation codon and the complement of the termination codon are underlined (SEQ ID NO:5)) to amplify the LT-coding sequence. The resulting PCR fragment was inserted into plasmid pJET1.2/blunt using the CloneJET PCR cloning kit (Thermo Fisher Scientific) to generate pJet1.2.LT-tsA58. pJet1.2.LT-tsA58 was incubated with MssI and EcoRI and the 2.5-kb digestion product was combined with the 11.0-kb MssI×EcoRI fragment of lentiviral vector shuttle plasmid pLV.iMHCK7 to generate pLV.iMHCK7.LT-tsA58. pLV.iMHCK7 is a derivative of pLVET-tTR-KRAB (Szulc et al., 2006; Addgene, Cambridge, Mass.; plasmid number: 116444) in which the human EEF1A1 gene promoter and the Aequorea victoria eGFP-coding sequence were replaced by the striated muscle-specific MHCK7 promoter (Salva et al., 2007) and a small polylinker containing unique SpeI, MssI, PstI and EcoRI restriction enzyme recognition sequences.

Mammalian expression plasmid pU.CAG.eGFP, in which eGFP expression is driven by the CAG promoter (Niwa et al., 1991), was generated by replacing the human TBX5-encoding SalI×NotI fragment of pU.CAG.hTBX5 (van Tuyn et al., 2007) with the eGFP-encoding SalI×NotI fragment of Clonetech's pEGFP construct (Takara Bio Europe, Saint-Germain-en-Laye, France).

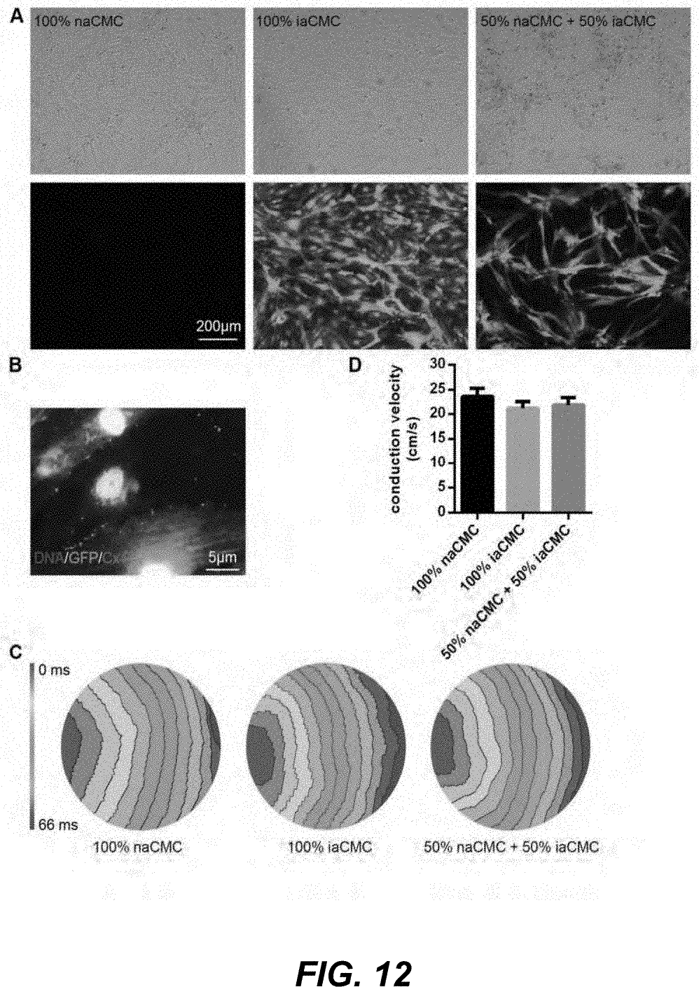

Lentiviral vector shuttle plasmid pLV.MHCK7.eGFP (FIG. 15) is a derivative of construct pLV.MHCK7.eYFP.WHVPRE (Bingen et al., 2014), in which the coding sequence for the Aequorea victoria enhanced yellow fluorescent protein and WHVPRE are replaced by the eGFP open reading frame and a mutated shortened version of the WHVPRE designated WHVoPRE (Schambach et al., 2006).

### Lentiviral Vector Production

To generate vesicular stomatitis virus G protein-pseudotyped particles of LV.iMHCK7.LT-tsA58, subconfluent monolayers of 293T cells were transfected with lentiviral vector shuttle construct pLV.iMHCK7.LT-tsA58 and the packaging plasmids psPAX2 (Addgene; plasmid number: 12260) and pLP/VSVG (Thermo Fisher Scientific) at a molar ratio of 2:1:1. The 293T cells were cultured in high-glucose Dulbecco's modified Eagle's medium (DMEM; Life Technologies Europe, Bleiswijk, The Netherlands; catalogue number: 41966) with 10% fetal bovine serum (FBS; Life Technologies Europe). The transfection mixture that consisted of 35 μg of plasmid DNA and 105 μg of polyethyleneimine (Polysciences Europe, Eppelheim, Germany) in 2 ml of 150 mM NaCl per 175-cm² cell culture flask (Greiner Bio-One, Alphen aan den Rijn, The Netherlands), was directly added to the culture medium. The next morning, the transfection medium was replaced by 15 ml of fresh high-glucose DMEM supplemented with 5% FBS and 25 mM HEPES-NaOH (pH 7.4). At ±48 hour after the start of the transfection procedure, the culture supernatants were harvested and cleared from cellular debris by centrifugation at room temperature (RT) for 10 minutes at 3,750×g and subsequent filtration through 0.45-μm pore-sized, 33-mm diameter polyethersulfone Millex-HP syringe filters (Millipore, Amsterdam, The Netherlands). To concentrate and purify the lentiviral vector particles, 30 ml of vector suspension in a 38.5-ml polypropylene ultracentrifuge tube (Beckman Coulter Nederland, Woerden, The Netherlands) was underlayed with 5 ml of 20% (wt/vol) sucrose in phosphate-buffered saline (PBS) and centrifuged for 120 minutes at 4° C. with slow acceleration and without braking at 15,000 revolutions per minutes in an SW32 rotor (Beckman Coulter Nederland). Next, the supernatants were discarded and the pellets were suspended in PBS-1% bovine serum albumin (BSA; Sigma-Aldrich, St. Louis, Mo.) by overnight incubation with gentle shaking at 4° C. The concentrated vector suspension was divided on ice in 50 μl aliquots for storage at −80° C. LV.MHCK7.eGFP particles were generated using the same procedure except for the use of pLV.MHCK7.eGFP instead of LV.iMHCK7.LT-tsA58 as lentiviral vector shuttle plasmid.

### Animal Experiments

All animal experiments were approved by the Animal Experiments Committee of the Leiden University Medical Center and conformed to the Guide for the Care and Use of Laboratory Animals as stated by the US National Institutes of Health.

### Isolation and Culture of Cardiomyocytes

naCMCs were isolated from neonatal Wistar rat hearts and cultured as detailed elsewhere (Feola et al., 2016). Briefly, rats were anaesthetized with 4-5% isoflurane inhalation. After adequate anesthesia had been confirmed by the absence of pain reflexes, hearts were excised. Atria were separated from the hearts, minced and dissociated by 2 subsequent 30-minute treatments with collagenase type I (Worthington Biochemical, Lakewood, N.J.) and DNase I (Sigma-Aldrich). Cells were pelleted by centrifugation for 10 minutes at RT and 160×g and suspended in Ham's F10 medium (Life Technologies Europe; catalogue number: 11550) supplemented with 100 units/mL of penicillin and 100 μg/mL of streptomycin (Life Technologies Europe) and with 10% heat-inactivated FBS and 10% heat-inactivated horse serum (HS; Life Technologies Europe). Next, cell suspensions were transferred to Primaria culture dishes (Corning Life Sciences, Amsterdam, The Netherlands) and incubated for 120 minutes at 37° C. in a humidified atmosphere of 5% CO₂ to allow preferential attachment of non-cardiomyocytes. The unattached cells (mainly naCMCs) were collected, passed through a cell strainer (70-μm mesh pore size; BD Biosciences, Breda, The Netherlands) and seeded at a density of 2×10³/cm² in a 6-well cell culture plate (Corning Life Sciences) coated with fibronectin from bovine plasma (Sigma-Aldrich) for immortalization purposes or on bovine fibronectin-coated glass coverslips in 24-well cell culture plates (Greiner Bio-One) at a density of 8×10⁵ cells/well for comparative studies. LV.iMHCK7.LT-tsA58-transduced cells and naCMCs were subsequently maintained in a 1:1 mixture of low-glucose DMEM (Life Technologies Europe; catalogue number: 22320) and Ham's F10 medium (DMEM/F10) supplemented with 5% heat-inactivated HS, 1×penicillin-streptomycin, 2% BSA and sodium ascorbate to a final concentration of 0.4 mM and in DMEM/F10 with 10% heat-inactivated FBS, 1× penicillin-streptomycin and 100 ng/ml Dox or no Dox as indicated, respectively. Culture medium was replaced daily (naCMCs) or every 2-3 days (iaCMCs).

For the isolation and culture of nvCMCs the same method was used as for naCMCs except that ventricular instead of atrial tissue of newborn rats served as starting material.

HL-1 cells were cultured in 25-cm² cell culture flasks (Corning Life Sciences) for regular passaging and on round glass coverslips (15 mm diameter) for optical mapping purposes essentially as described in the MEA Application Note: HL-1 Cardiac Cell Line of Multi Channel Systems (Reutlingen, Germany; http://www.multichannelsystems.com/sites/multichannelsystems.com/files/documents/applications/MEA-Application %20Note_HL-1.pdf).

Transduction of naCMCs and Generation of iaCMC Clones

At day 1 of culture, naCMCs were transduced with LV.iMHCK7.LT-tsA58. The next day, the inoculum was replaced by culture medium containing 100 ng/ml Dox to induce LT expression. The cells were given fresh culture medium every other day. After 1 week of culture, the transduced cells were trypsinized and plated at a low density of 10-20 cells/cm² in a 100-mm diameter cell culture dish (Greiner Bio-One) to generate single-cell clones. After 2-3 weeks, individual cell colonies were picked and expanded in the presence of Dox. To investigate their spontaneous cardiomyogenic differentiation ability, iaCMCs were cultured in the absence of Dox for 3, 6, 9 and 12 days and characterized by a combination of molecular biological, cell biological, immunological and electrophysiological techniques.

To facilitate their identification in co-cultures with naCMCs and after injection into atrial tissue, LV.iMHCK7.LT-tsA58-transduced iaCMCs were eGFP-labeled using the lentiviral vector designated LV.MHCK7.eGFP and passaged multiple times in the presence of Dox before use as donor cells to avoid carry-over of functional LV.MHCK7.eGFP particles (Ramkisoensing et al., 2012).

### Western Blotting

Western blotting was carried out as detailed before (Majumder et al., 2016). After blocking, the membranes were incubated overnight at 4° C. with primary mouse monoclonal antibodies directed against LT (1:2,000; Santa Cruz Biotechnology, Dallas, Tex., catalogue number: sc-147) or GAPDH (loading control; 1:100,000; Millipore, catalogue number: MAB374) and then probed with goat anti-mouse IgG secondary antibodies linked to horseradish peroxidase (1:15,000; Santa Cruz Biotechnology) for 1 hour at RT. Chemiluminescence was produced using the SuperSignal West Femto maximum sensitivity substrate (Thermo Fisher Scientific), captured by a ChemiDoc XRS imaging system (Bio-Rad Laboratories, Veenendaal, The Netherlands) and analyzed by Quantity One software (Bio-Rad Laboratories) using the GAPDH signals for normalization purposes.

### Immunocytology

Immunostaining of cultured cells was performed as previously described (Yu et al., 2016) using the following primary antibodies at a dilution of 1:200 in PBS containing 0.1% normal donkey serum (NDS; Sigma-Aldrich): mouse anti-LT (Santa Cruz Biotechnology, catalogue number: sc-147), rabbit anti-Ki-67 (Abcam, catalogue number: ab15580), mouse anti-α-actinin (Sigma-Aldrich, catalogue number: A7811), rabbit anti-MLC2a (Kubalak et al., 1994), rabbit anti-Cx43 (Sigma-Aldrich, catalogue number: C6219) and rabbit anti-ERG1/Kᵥ11.1 (Millipore, catalogue number: AB5930). Bound antigens were detected using Alexa 488-conjugated donkey anti-rabbit IgG (H+L) (Thermo Fisher Scientific, catalogue number: A21206) and Alexa 568-coupled donkey anti-mouse IgG (H+L) (Thermo Fisher Scientific, catalogue number: A10037). These secondary antibodies were diluted 1:400 in PBS. Hoechst 33342 (10 μg/μl; Thermo Fisher Scientific) was used to counterstain nuclei. Stained cells were visualized using a digital color camera-equipped fluorescence microscope (Nikon Eclipse 80i; Nikon Instruments Europe, Amstelveen, The Netherlands).

### Analysis of Cell Proliferation and Programmed Cell Death

To assess their proliferation rate, iaCMCs were cultured at low density in medium with or without 100 ng/ml Dox. At different days after culture initiation, cells were collected in PBS, mixed at a ratio of 1:1 with 0.4% Trypan Blue (VWR International, Amsterdam, The Netherlands) in PBS and briefly incubated at RT, after which viable cells were counted using a hemocytometer. To distinguish proliferating from non-proliferating cells, monolayer cultures of naCMCs were immunostained with Ki-67-specific antibodies.

To study apoptosis, cells were incubated with Alexa Fluor-568-conjugated annexin V (Thermo Fisher Scientific, catalogue number: A13202) as described before (Yu et al., 2016). nvCMCs treated for 24 hour with 1 μM of the chemotherapeutic agent doxorubicin (Sigma-Aldrich) served as positive control for the apoptosis assay.

### RT-qPCR

RT-qPCR was performed as previously reported (Yu et al., 2016). Briefly, iaCMCs were seeded at a density of 8×10⁵ cells/well in 24-well cell culture plates and cultured for 1 day in medium containing 100 ng/ml Dox. The next day, cells were either lysed in TRIzol Reagent (Thermo Fisher Scientific) and total RNA was isolated using the RNeasy Mini kit (QIAGEN Benelux, Venlo, The Netherlands) or kept for 3, 6, 9 or 12 days in culture medium without Dox prior to cell lysis and RNA extraction. The RNA was reverse transcribed with the iScript cDNA synthesis kit (Bio-Rad Laboratories) and gene expression levels were determined by PCR using the Bioline SensiFAST SYBR No-ROX kit (GC biotech) and intron-spanning primer pairs (Table 1). PCR amplifications were performed in a CFX96 Touch Real-Time PCR Detection System (Bio-Rad Rad Laboratories). Per target gene 3 independent experiments were performed consisting of 3 samples per experiment. Quantitative analyses were based on the 2^{-ΔΔCT} method using CFX Manager software (Bio-Rad Rad Laboratories). PCR amplifications of the rat 18S rRNA gene (Rn18s) were included for normalization purposes.

TABLE 1

| Gene | Forward primer | Reverse primer |
|---|---|---|
| Actn2 | GGCTATGAGGAATGGCTATTGA | AAGTAGGGCTCGAACTTCC |
| | (SEQ ID NO: 7) | (SEQ ID NO: 8) |
| Atp2a2 | TGACCCACGAGCTGTTAATC | GGTGTTCTCTCCTGTTCTGT |
| | (SEQ ID NO: 9) | (SEQ ID NO: 10) |
| Cacna1c | CAAGGTGGTACACGAAGCTCA | ACAGTGCTGCCCCTGGAGTA |
| | (SEQ ID NO: 11) | (SEQ ID NO: 12) |
| Gata4 | GGCTCTCTGGGAAACTGG | GAGGTGCCTAGTCCTTGC |
| | (SEQ ID NO: 13) | (SEQ ID NO: 14) |
| Kcnd3 | CAACTTTAGCAGGATCTACCATC | AGAGCTTCATTGAGGAGTCCA |
| | (SEQ ID NO: 15) | (SEQ ID NO: 16) |
| Kcnh2 | TAGCCTCCTCAACATCCC | CCATGTCTGCACTTAGCC |
| | (SEQ ID NO: 17) | (SEQ ID NO: 18) |
| Kcnj2 | GGATCTTACATGCTTCTGTAACC | CAGAGAACTTGTCCTGTTGC |
| | (SEQ ID NO: 19) | (SEQ ID NO: 20) |
| Kcnj3 | ACAGCCACATGGTCTCC | CCAGTTCAAGTTGGTCAAGG |
| | (SEQ ID NO: 21) | (SEQ ID NO: 22) |
| Kcnj5 | GGTTGTGGTCATACTAGAAGGG | GCAGTCATATCTGCCTTGGG |
| | (SEQ ID NO: 23) | (SEQ ID NO: 24) |
| Kncj11 | GGAAGACCCGGAGGTAATAAG | CTCCACTCAGCTATCCTTCAC |
| | (SEQ ID NO: 25) | (SEQ ID NO: 26) |
| Mef2c | GATTCAGATCACGAGGATTATGG | GTGCTGTTGAAGATGATCAGG |
| | (SEQ ID NO: 27) | (SEQ ID NO: 28) |
| Myh6 | CTACCAGATCCTGTCCAACAA | GCACATCAAAGGCGCTATC |
| | (SEQ ID NO: 29) | (SEQ ID NO: 30) |
| Myh7 | CACCAAGAGGGTCATCCAATA | CCAAATCGGGAGGAGTTATCA |
| | (SEQ ID NO: 31) | (SEQ ID NO: 32) |
| Myl7 | TCAATGTTCGAGCAAGCC | CTGACTTGCAGATGATTCCG |
| | (SEQ ID NO: 33) | (SEQ ID NO: 34) |
| Nkx2.5 | CTCCCACCTTTAGGAGAAGG | CGAGGCATCAGGTTAGGTC |
| | (SEQ ID NO: 35) | (SEQ ID NO: 36) |
| Nppa | CCAAGGGCTTCTTCCTCTTC | CTCATCTTCTACCGGCATCTTC |
| | (SEQ ID NO: 37) | (SEQ ID NO: 38) |
| Rn18s | GTAACCCGTTGAACCCCATT | CCATCCAATCGGTAGTAGCG |
| | (SEQ ID NO: 39) | (SEQ ID NO: 40) |
| Ryr2 | AACTGGCCTTTGATGTTGGC | AATATGAGGTCGTCTCCAACCC |
| | (SEQ ID NO: 41) | (SEQ ID NO: 42) |
| Scn5a | CCATTGTGCCCTGACGAACCTTA | GCTGCCTCCAGCTTCCACACA |
| | (SEQ ID NO: 43) | (SEQ ID NO: 44) |

### Patch Clamping

Patch-clamp recordings were conducted at 20-23° C. in naCMCs of culture day 2 to 4 and in iaCMCs that had been maintained for 3, 6, 9 and 12 days in Dox-free culture medium. In brief, cells were transferred from culture medium to a bath solution containing (in mM): 126 NaCl, 11 glucose, 10 HEPES, 5.4 KCl, 1 MgCl₂ and 1.8 CaCl₂ (adjusted to pH 7.40 with NaOH). Within 15-60 minutes after exposure to bath solution, whole-cell patch-clamp recordings were made with the aid of an inverted Zeiss Axiovert 35 microscope (Carl Zeiss, Oberkochen, Germany), a MPC-200 multi-micromanipulator system (Sutter Instrument, Novato, Calif.) and a conventional patch-clamp rig consisting of a CV-7B head stage, MultiClamp 700B amplifier and a Digidata 1440A A/D converter (Axon CNS, Molecular Devices, Sunnyvale, Calif.). The patch pipettes were fabricated from borosilicate glass capillaries with an inner and outer diameter of 1.17 and 1.5 mm, respectively (Harvard Apparatus, Kent, United Kingdom), using a vertical puller (P-30; Sutter Instrument). Pipettes had typical electrical resistances of 2-3 MΩ in bath solution when filled with a solution containing (in mM): 80 potassium DL-aspartate, 40 KCl, 8 NaCl, 5.5 glucose, 5 HEPES, 5 EGTA, 1 MgCl₂, 4 Mg-ATP and 0.1 Na₃-GTP (adjusted to pH 7.20 with KOH). The GΩ seal was formed by applying 10-ms voltage steps from 0 to +5 mV at 10 Hz. After reaching GΩ seal, the holding potential was set to −50 mV, and the patch membrane was ruptured by gentle suction. Whole-cell capacitance was calculated from capacitive transient currents evoked by raising the transmembrane potential in 5-mV steps starting from a holding potential of −50 mV and these currents were cancelled with the amplifier. To minimize voltage errors and hence optimize the accuracy of the voltage clamp, pipette series resistance was electrically compensated by >75%.

In the actual experiments to assess the excitability and some basic electrophysiological properties of the iaCMCs, the voltage and current commands were delivered to the MultiClamp 700B amplifier via the Digidata 1440A A/D converter. The latter instrument was controlled by MultiClamp 700B commander and pCLAMP 10 software (Axon CNS, Molecular Devices). Throughout the experiments, the current and voltage outputs of the amplifier were continuously sampled at intervals of 100 s and recorded onto a personal computer after low-pass filtering at 4 kHz with a four-pole Bessel filter. For data analysis, the estimated liquid junction potential of 11 mV was corrected.

### Optical Voltage Mapping

For studying AP propagation in confluent monolayer cultures, cells were seeded at a density of 8×10⁵ cells/well in 24-well cell culture plates onto bovine fibronectin-coated round glass coverslips (15 mm diameter) and subjected to optical voltage mapping using di-4-ANEPPS (Thermo Fisher Scientific) as fluorescent voltage indicator as described previously (Bingen et al., 2014). The measurements were carried out at 37° C. on 9-day-old naCMC and nvCMC cultures and on iaCMCs that had been maintained for 0, 3, 6, 9 and 12 days in Dox-free medium. Optical signals were captured using a MiCAM ULTIMA-L imaging system (SciMedia, Costa Mesa, Calif.). Optical traces were analyzed using Brain Vision Analyzer 1208 software (Brainvision, Tokyo, Japan). To minimize noise artifacts, calculations were based on the average of the signals at a selected pixel and its eight nearest neighbors. CV and APD at different percentages of repolarization were determined using cardiomyocyte cultures showing uniform AP propagation and 1:1 capture after 1-Hz local electrical stimulation with an epoxy-coated bipolar platinum electrode delivering square 10-ms, 8-V suprathreshold electrical impulses via a STG 2004 stimulus generator and MC Stimulus II software (both from Multi Channel Systems). Burst pacing with a cycle length of 20 to 100 ms was used to induce reentry. The effect of tertiapin (100 nM, Alomone Labs, Jerusalem, Israel) and astemizole (100 nM, a gift of Zhiyi Yu) was investigated by pipetting them directly into the medium and dispersing them by gentle shaking, followed by optical mapping. To facilitate comparison of the APD, the amplitude of the optical signal traces was normalized for all samples within a given experiment.

Atrial Tissue Culture and iaCMC Injection in Cultured Atrial Tissue

Atrial tissue from neonatal rats was used as graft recipient of iaCMCs. Two-day-old Wistar rats were anaesthetized by 4-5% isoflurane inhalation and adequate anesthesia was confirmed by the absence of reflexes. After the chest was opened and the inferior vena cava was cut off with scissors, the right atrium was punctured with a 19-gauge needle. The heart was perfused through this needle with pre-warmed and oxygenated PBS containing 10 IU/ml heparin to flush out the blood. Two and a half μl of growth factor-reduced Matrigel (BD Biosciences) was mixed with 2.5 μl cell suspension containing 5×10⁴ iaCMCs and injected into the bilateral atrial free walls using a 25-μl syringe equipped with a 33-gauge needle (Hamilton Robotics, Reno, Nev.).

Subsequently, the atrial tissue including the injected area was excised and cultured as described by Brandenburger et al. (2012) with the following modifications. The atrial tissue was placed with the endocardial surface down onto the semi-porous polytetrafluoroethylene membrane (0.4 μm pore size) of a PICMORG50 cell culture insert (Millipore) in a 35-mm diameter Petri dish containing 1 ml sterilized Tyrode's solution (TS; 136 mM NaCl, 5.4 mM KCl, 10 mM MgH₂PO₄, 1 mM MgH₂PO₄, 10 mM glucose, 0.9 mM CaCl₂) of RT and gradually warmed to 37° C. Next, the TS was replaced with 1 ml pre-warmed low-glucose DMEM supplemented with 1% heat-inactivated FBS, 1× penicillin-streptomycin, 1× B-27 supplement minus antioxidants (Life Technologies Europe) and 5 μM Z-Asp-2,6-dichlorobenzoyloxymethylketone (Santa Cruz Biotechnology) and the tissue was kept at 37° C. in humidified 95% air-5% CO₂ (culture conditions) until histological examination. Culture medium was replaced every 2 days.

### Whole Tissue Staining and Confocal Microscopy

Neonatal rat atrial tissue was quickly washed 3 times with PBS and fixed by incubation for 30 minutes at 4° C. in buffered 4% formaldehyde (Added Pharma, Oss, The Netherlands) followed by 3 quick washes with PBS and a 5-minute wash with PBST (0.1% Triton X-100 [Sigma-Aldrich] in PBS). Next, aspecific binding sites were blocked by incubation for 1 hour at RT with 10% NDS in PBST. After two 10-minute washes with PBST, samples were incubated overnight at 4° C. with primary antibodies directed against sarcomeric α-actinin to detect cardiomyocytes (mouse IgG1, clone EA-53; Sigma-Aldrich) or against eGFP to detect iaCMCs (rabbit IgG, polyclonal; Thermo Fisher Scientific, catalogue number: A11122) and diluted 1:200 in PBS-1% FBS. After 3 quick and four 15-minute washes with PBS, the tissues were incubated with appropriate Alexa Fluor 488/568-conjugated secondary antibodies of donkey origin (1:200 dilution in PBS containing 1% FBS) for 4 hour at 4° C. followed by another sequence of 3 short and 4 long washes with PBS. Nuclear counterstaining was performed by incubating the cells for 10 minutes at RT with 10 μg/ml Hoechst 33342 followed by two quick and one 5-minute wash with PBS. Coverslips were mounted in Vectashield mounting medium (Vector Laboratories, Burlingame, Calif.). Images were acquired with a Leica TCS SP8 confocal laser scanning microscope (Leica Microsystems, Rijswijk, The Netherlands). For Z plane reconstruction, confocal stacks with a step size of 0.3 μm were acquired and the resulting images were analyzed using Leica Application Suite AF Lite software (Leica Microsystems).

Plasmid DNA Transfection of iaCMCs.

For monitoring the transfection efficiency of iaCMCs with plasmid DNA, the eGFP-encoding mammalian expression plasmid pU.CAG.eGFP was used. One day before transfection, iaCMCs were seeded at a density of 4.8×10⁵ cells per well in a 24-well cell culture plate and maintained in iaCMC culture medium with Dox. The next day, the cells were transfected with Lipofectamine 3000 reagent (Thermo Fisher Scientific) according to the manufacturer's instructions. To this end, 50 μl of plasmid DNA-lipid complexes containing 500 ng of pU.CAG.eGFP DNA, 1 μl of P3000 reagent and 1.5 μl of Lipofectamine 3000 reagent diluted in Opti-MEM I reduced serum medium (Thermo Fisher Scientific) were added per well to 500 μl of iaCMC culture medium without Dox. At the indicated times after the start of the transfection procedure, the cells were processed for flow cytometry using a BD Accuri flow cytometer (BD Biosciences), (immuno)fluorescence microscopy and optical voltage mapping. For the cells that were analyzed at day 9 post-transfection, the transfection medium was replaced with regular iaCMC culture medium without Dox 2 days after the addition of the lipoplexes.

### Statistical Analysis

Data were derived from >3 biological replicates. Whenever appropriate, data were expressed as mean±SD for a specified number (N) of observations and SDs were represented by error bars. Data were analyzed with the aid of GraphPad Prism version 6 software (Graphpad Software, La Jolla, Calif.) using the Mann-Whitney-Wilcoxon test for all analyses. Results were considered statistically significant at P values <0.05. Statistical significance was expressed as follows: *: P<0.05, **: P<0.01, ***: P<0.001, ****: P<0.0001 and NS: not significant.

### Results

Conditional Immortalization of naCMCs

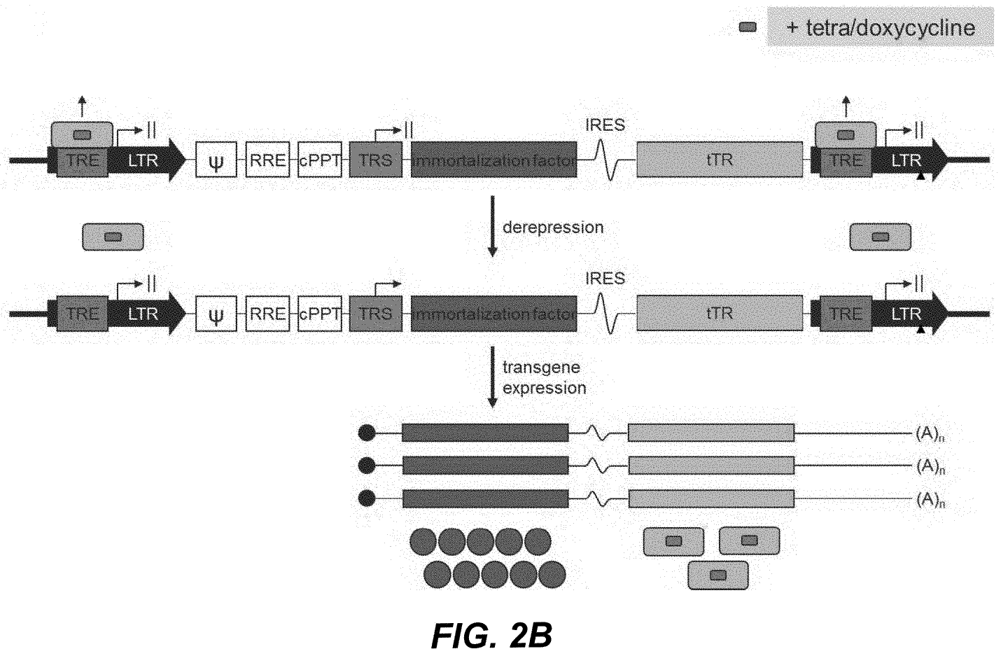

As starting material for the conditional immortalization of naCMCs, low-density cardiomyocyte-enriched cultures of primary cells isolated from the atria of 2-day-old Wistar rats was used. These cultures were transduced with lentiviral vector particles containing a Dox-inducible LT expression unit based on the monopartite lentiviral Tet-on system developed by Szulc et al. (2006). LT expression in this lentiviral vector system was driven by the strong hybrid striated muscle-specific MHCK7 promoter (Salva et al., 2007; FIG. 3, Panel A) to minimize the likelihood of inadvertently immortalizing the ±12% non-cardiomocytes present in the primary atrial cell cultures (data not shown; Bingen et al., 2013).

In the presence of Dox, cells started to divide forming clearly discernable cell clones at 1-3 weeks post-transduction (FIG. 3, Panel B). Nine of >80 clones were randomly selected for testing the Dox dependency of their proliferation ability. Cells of all 9 clones were actively dividing in the presence of Dox and stopped doing so in its absence (data not shown). To investigate whether these clones contained excitable cells, confluent monolayers were established, maintained in Dox-free culture medium for 9 days and examined by optical voltage mapping. Following 1-Hz electrical stimulation, 7 of the 9 clones produced excitatory waves spreading from the pacing electrode to the opposite site of the culture dishes. Of these 7 clones, clone #5 showed the shortest APD and highest CV in this preliminary screen and was therefore picked for further investigation. Western blot analysis and immunofluorescence microscopy showed expression of LT in this clone to be tightly and effectively controlled by Dox (FIG. 3, Panels C-E).

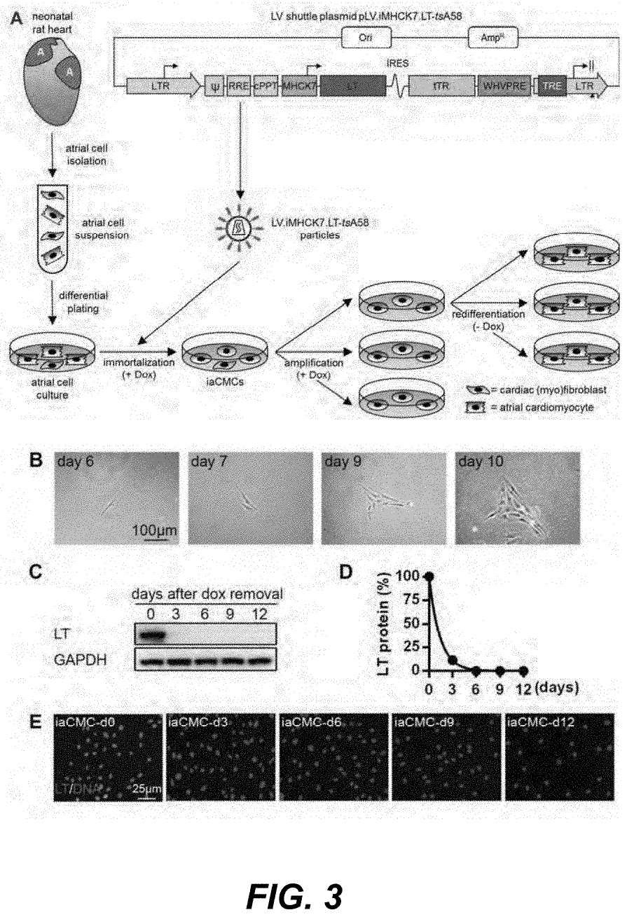

Comparison of the light microscopic morphology of the iaCMCs with those of naCMCs revealed that proliferating iaCMCs (iaCMC-d0) were marginally smaller and less adherent than naCMCs at culture day 1, whereas in near-confluent monolayers iaCMCs that were kept for 9 days in Dox-free medium very closely resembled naCMCs at culture day 9 in size and appearance (FIG. 4, Panel A).

In the presence of Dox, 72.2±8.1% of iaCMCs stained positive for the proliferation marker Ki-67, which percentage dramatically decreased upon Dox removal (FIG. 4, Panels B and C). Consistently, in the presence of Dox, iaCMCs proliferated with an average doubling time of 38 hour, whereas under differentiation conditions (i.e., in the absence of Dox), the cell number slightly decreased with time (FIG. 4, Panel D). As expected, in primary atrial cell cultures, the percentage of α-actinin⁺ cells expressing Ki-67 was very low indicating that the large majority of proliferating, Ki-67⁺ cells in these cultures are non-cardiomyocytes (FIG. 4, Panels C and E). Probing iaCMC cultures with fluorescently labeled annexin V for the presence of surface-exposed phosphatidylserine revealed only a low percentage of apoptotic cells in proliferating cultures, which slightly increased under differentiation conditions (FIG. 4, Panel F) but did not noticeably differ from the percentage of apoptotic cells in 9-day-old naCMC cultures (FIG. 4, Panel G).

Cardiomyogenic Differentiation Potential of iaCMCs

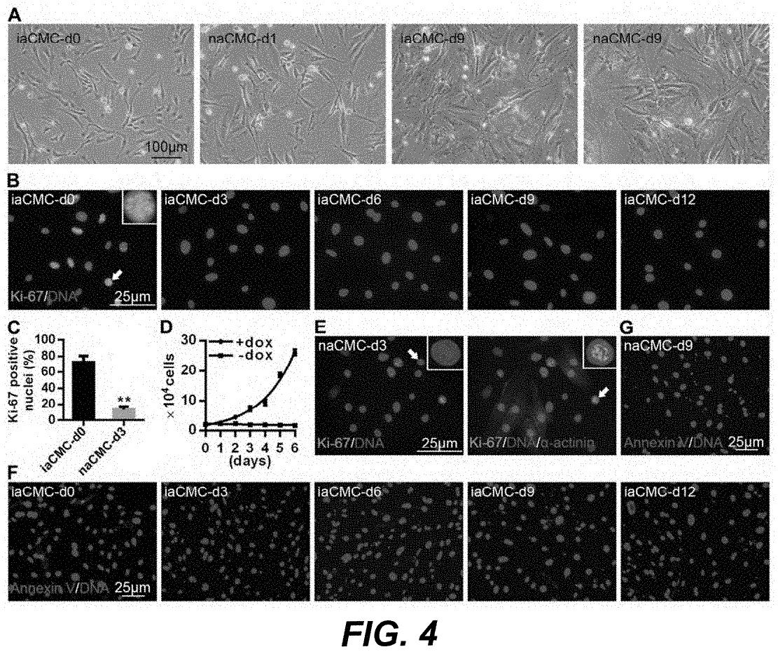

To investigate the cardiomyogenic differentiation potential of iaCMCs, the expression of cardiac marker genes in proliferating and differentiating iaCMC cultures was assessed by RT-qPCR analysis and compared with that of naCMCs and of native nvCMCs at culture day 9. Expression of the Nkx2.5, Gata4 and Mef2c genes that encode early cardiac transcriptional regulators, was higher in proliferating (i.e., day 0) iaCMCs than in the naCMCs and nvCMCs and rapidly increased soon after Dox removal (FIG. 5A). Expression of the sarcomeric protein-encoding Actn2 and Myh6 genes, on the other hand, was lower in day 0 iaCMCs than in the naCMCs and nvCMCs. Transcription of these genes also showed a rapid increase under differentiation conditions to very similar levels as in naCMCs and nvCMCs (FIG. 5A).

Next, to study myofibrillogenesis, cells were immunostained for sarcomeric α-actinin (FIG. 5B). At the immortalized stage, α-actinin expression in iaCMCs was low and in most cells the protein was diffusely spread throughout the cytoplasm. However, in a few cells, α-actinin showed a punctuate staining pattern with the protein clustering in spherical or short elongated structures. At day 3 of differentiation, α-actinin formed longer structures with a dot-like appearance reminiscent of immature myofibrils. Myofibril assembly nearly reached completion at day 6 after Dox removal, with most of the α-actinin displaying the Z-line staining pattern typical of cardiomyocytes. At the later stages of differentiation (i.e., at days 9 and 12 after Dox removal), myofibrils underwent further maturation resulting in cells with highly organized sarcomeres.

After Dox removal, mRNA levels of the atrial natriuretic peptide-encoding Nppa gene and the atrial myosin light chain 2 (Mlc2a)-encoding Myl7 gene were strongly up-regulated in iaCMCs (FIG. 5C). Moreover, neither in the absence nor in the presence of Dox, iaCMCs expressed detectable amounts of β-myosin heavy chain-encoding Myh7 transcripts indicating that iaCMCs reacquire properties of atrial rather than ventricular cardiomyocytes when cultured in Dox-free medium (FIG. 5C). Mlc2a immunostaining confirmed these findings and showed gradual incorporation of this protein into sarcomeres indiscernible from those in naCMCs that had been cultured for 9 days (FIG. 5D). The functionality of these sarcomeres was demonstrated by the observation that iaCMCs started to exhibit spontaneous contractions from day 6 of differentiation. The immunostainings for α-actinin and Mlc2a also revealed that the spontaneous differentiation of the iaCMCs in the absence of Dox is a highly synchronous event occurring in essentially all cells at approximately the same time.

Electrophysiological Properties of iaCMCs

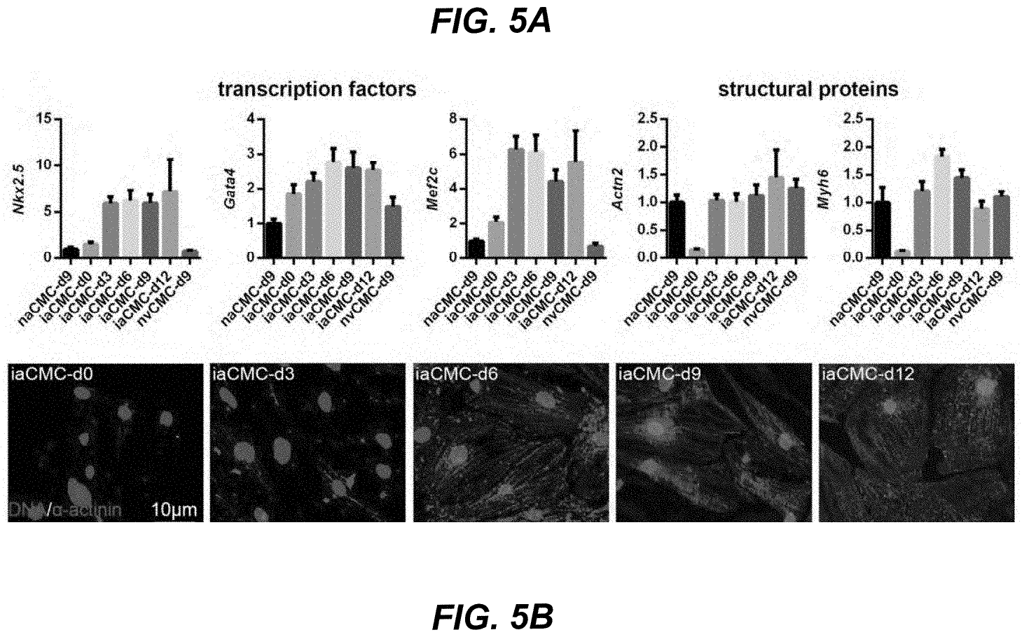

RT-qPCRs targeting genes encoding cardiac ion channels showed an increase in the mRNA levels of Scn5a/Nav1.5, Cacna1c/Cav1.2, Kcnj3/Kir3.1, Kcnj5/Kir3.4 and Kcnj11/Kir6.2 during the early stages of iaCMC differentiation (iaCMC-d3 and iaCMC-d6), which, later in the differentiation process (iaCMC-d9 and iaCMC-d12), dropped to approximately equal levels as in naCMCs at culture day 9 (FIG. 6, Panels A-E, respectively). A similar differentiation-dependent trend in expression levels was observed for Kcnd3/Kᵥ4.3 and Kcnh2/Kᵥ11.1 but in these cases transcript levels at the late stages of iaCMC differentiation (iaCMC-d9 and iaCMC-d12) were higher than those in day 9 naCMCs (FIG. 6, Panels F and G, respectively). The iaCMCs did not express detectable amounts of Kcnj2/Kir2.1 at any stage in the differentiation process in contrast to naCMCs that had been cultured for 9 days (FIG. 6, Panel H). Furthermore, Kcnj2 expression in naCMCs was much lower than in nvCMCs at culture day 9 and is consistent with previous findings (Zhang et al., 2013). RT-qPCR analysis of Ryr2 and Atp2a2/Serca2 transcripts showed an increase in the expression of these Ca²⁺-handling protein genes early during iaCMC differentiation. From day 3 of differentiation onwards, Ryr2 and Atp2a2 mRNA levels in iaCMCs remained constant and were very similar (Ryr2) or 2- to 3-fold higher (Atp2a2) than in naCMCs at culture day 9 (FIG. 6, Panels I and J).

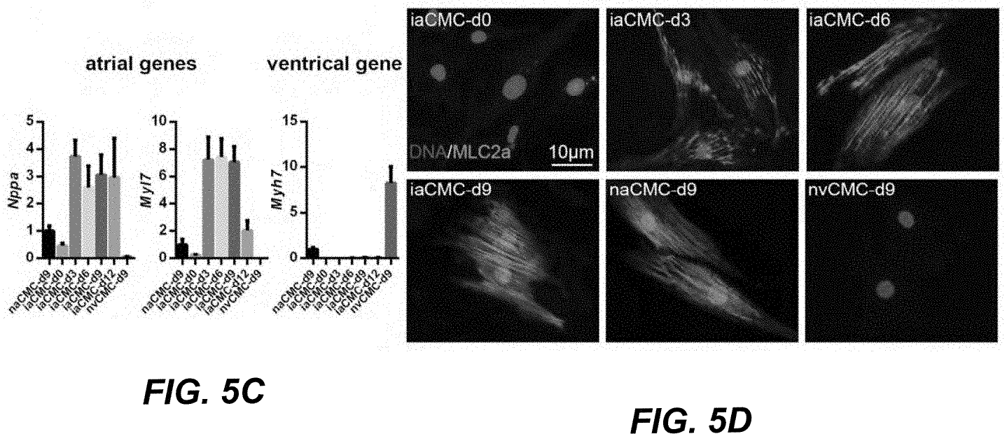

Solitary naCMCs at culture day 2-4 and iaCMCs maintained in the absence of Dox for different time periods were patch-clamped to study electrophysiological function. Short depolarizing current-clamp stimulations (10-20 ms, 100-300 pA) were sufficient to evoke APs in naCMCs and in a subpopulation of the iaCMCs (excitable iaCMCs) as shown in FIGS. 7A (Panel b) and 7B (Panel b). However, as depicted in FIG. 7C (Panel b), these current pulses failed to initiate APs in another subpopulation of iaCMCs (inexcitable iaCMCs), but rather exhibited largely passive decay of the input current. The fraction of excitable iaCMCs gradually increased from ±35% at day 3 of culture in the absence of Dox to ±90% at day 9 and 12 after the initiation of the differentiation process by Dox removal (FIG. 7D).

To investigate the ionic basic of the APs generated by iaCMCs, total membrane current was recorded using voltage steps applied from a holding potential of −90 mV to test potentials ranging from −130 through +70 mV. In naCMCs (FIG. 7A (Panel a)), the total current evoked by hyperpolarizing potentials was inward and stationary and had the properties of an inward rectifier current. The magnitude of this current gradually declined to zero moving from −130 to −70 mV. Weak depolarization beyond the putative resting membrane potential of −70 mV elicited a strong total inward current with characteristics typical of a fast Na⁺ current. Upon further depolarization, the total inward current became smaller and gradually gave way to a tiny outward current which, based on its characteristics and the RT-qPCR data, likely represents an outward rectifier K⁺ current. As shown in FIG. 7B (Panel a), excitable iaCMCs displayed a similar total inward current as naCMCs but a much stronger total outward current and little evidence of inwardly rectifying K⁺ current, consistent with the absence of detectable Kcnj2 expression in iaCMCs. The inexcitable iaCMCs did not produce a significant total inward current but they did show a large sustained total outward current (FIG. 7C (Panel a)). The average cell membrane capacitance of iaCMCs that had been cultured at low density for 3, 6, 9 and 12 days in the absence of Dox was 83.0±17.3, 108.9±38.5, 219.2±60.6 and 166.8±98.9 pF, respectively, as compared to 33.8±8.3 pF for naCMCs in 2- to 4-day old low density cultures (FIG. 7E). The larger membrane capacitance and therefore cell surface area of iaCMCs may relate to their more flattened shape and explain why depolarization of these cells was slower than that of naCMCs (compare FIG. 7A (Panel b) with FIG. 7B (Panel b)). Another possible contributor to the relatively low upstroke velocity of excitable iaCMCs might be the strong outward currents produced by these cells, counteracting the inward currents.

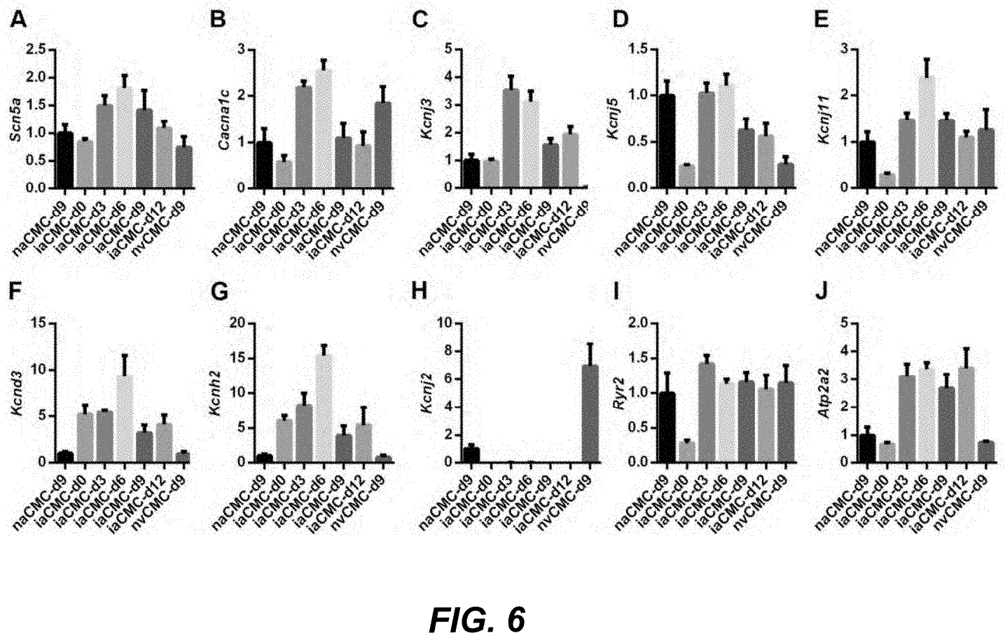

Since spreading of APs though cardiac tissue critically depends on the presence of intercellular conduits, iaCMC cultures at different stages of differentiation were immunostained for the gap junction protein connexin 43 (Cx43). As shown in FIG. 7F, Cx43 was first observed in iaCMCs at 3 days after Dox removal and its amounts gradually increased as the cells further differentiated. Initially, Cx43 was mainly detected in the perinuclear region (i.e., at the site of biosynthesis) but at days 9 and 12 of differentiation, most Cx43 was found in punctuate patterns at the interfaces between iaCMCs. To investigate the functionality of these gap junctional plaques, confluent monolayers of differentiated iaCMCs were subjected to 1-Hz electrical point stimulation that caused the cells to contract at the pacing frequency. This result was corroborated by optical voltage mapping that showed uniform radial spreading of excitatory waves from the site of electrical point stimulation in iaCMC cultures at day 9 of differentiation. Additional optical voltage mapping experiments revealed a gradual increase in CV (FIGS. 7G and 7I) and decrease in APD (FIGS. 7H, 7J and 7K) with ongoing iaCMC differentiation. As a matter of fact, CV APD₃₀ and APD₈₀ did not significantly differ between iaCMC cultures at differentiation days 9 and 12 (CV=20.8±1.9 cm/s; APD₃₀=16.8±1.3 ms; APD₈₀=54.3±5.0 ms) and 9-day-old naCMC cultures (CV=22.4±1.9 cm/s; APD₃₀=16.9±1.0 ms; APD₈₀=54.0±2.8 ms). These data suggest that upon Dox removal, iaCMCs differentiate into atrial myocytes with electrophysiological properties strongly resembling those of naCMCs. Optical voltage mapping of other clones of excitable iaCMCs yielded very similar results although not all of them reached the same maximum CV and minimum APD as clone #5 (e.g., clone #1 and #6; FIG. 8, Panels A-C and D-F, respectively).

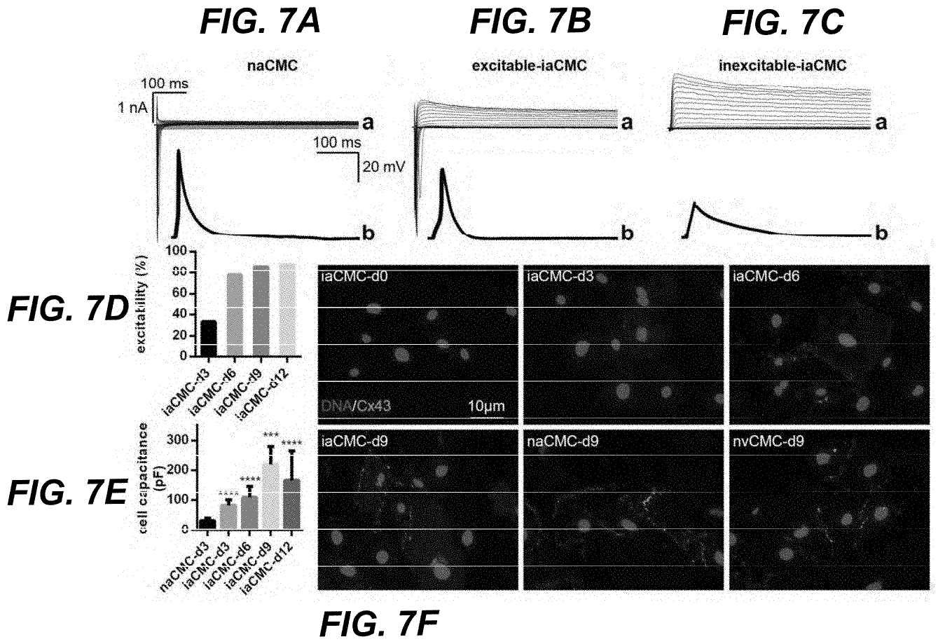

Finally, a comparison was made between the electrophysiological properties of iaCMC clone #5 and those of the widely used HL-1 cell line that was derived from the AT-1 mouse atrial cardiomyocyte tumor lineage (Claycomb et al., 1998). To this end, confluent monolayers of iaCMCs at day 9 of differentiation and optimally maintained confluent cultures of HL-1 cells were subjected to optical voltage mapping following 1-Hz pacing. The CV in the HL-1 cultures was ±20-fold lower than in the iaCMC cultures (FIG. 9, Panels A and B) and HL-1 cells displayed greatly prolonged APs as compared to differentiated iaCMCs (FIG. 9, Panel C) characterized by a very slow upstroke. These data clearly demonstrate that differentiated iaCMCs possess electrophysiological properties far superior to those of HL-1 cells.

iaCMCs as In Vitro Model of AF

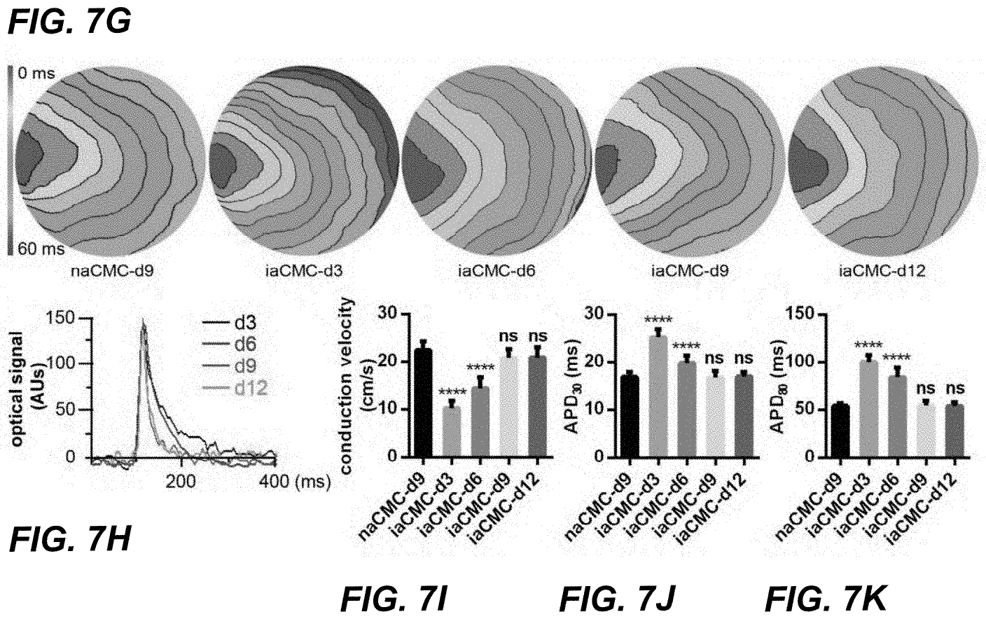

Our research group previously developed an in vitro model of AF based on high-frequency electrical stimulation of confluent naCMC cultures. The resulting reentrant circuits, or so-called rotors, could be terminated by prolonging APD of the naCMCs using the Kir3.x-specific inhibitor tertiapin (Bingen et al., 2013). To determine whether confluent cultures of iaCMCs at different stages of differentiation would display similar behavior, they were burst paced in the absence or presence of 100 nM tertiapin. While rotors could be readily induced in iaCMC cultures from differentiation day 6 onwards (FIG. 10 Panels A-C), reentry could not be established in iaCMC cultures at day 3 of differentiation (FIG. 10, Panel C). Reentry inducibility, the number of rotors per culture and rotor frequency increased with the time after Dox removal (FIG. 10, Panels C-E). After treatment with tertiapin, iaCMCs that had been cultured for 9 days without Dox showed a strong increase in APD (FIG. 10, Panels F-H). Moreover, in the presence of tertiapin it was no longer possible to induce reentry in confluent monolayers of iaCMCs at day 6, 9 or 12 of differentiation by burst pacing (FIG. 10, Panels C-E and F). The fact that tertiapin treatment only slightly reduced the CV in confluent monolayers of iAMs at day 9 of differentiation (from 20.5 to 19.8 cm/s) suggests that Kir3.x channel activity is not the major determinant of iAM excitability.

iaCMCs as In Vitro Model for Drug Screening

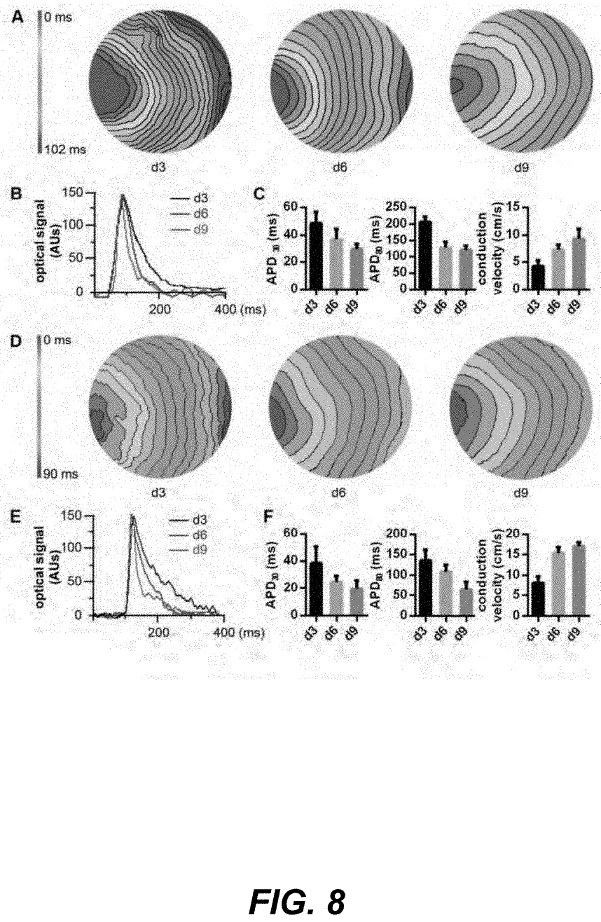

Drug-induced ventricular arrhythmias due to inadvertent blockade of the Kᵥ1.1 channel that is encoded by the Kcnh2 gene, represents a major safety concern in drug development (Kannankeril et al., 2010). RT-qPCR analysis showed that the Kcnh2 gene is highly expressed in iaCMCs peaking at 6 days after the initiation of differentiation. At this time, Kcnh2 transcripts were 16-fold more abundant in iaCMCs than in naCMCs at culture day 9 (N=3, P<0.01; FIG. 6, Panel G). Double immunostaining of these cells for Kᵥ11.1 and sarcomeric α-actinin showed that both cell types expressed the Kᵥ11.1 protein and that it was concentrated around the nucleus and at the sarcolemma (FIG. 11, Panel A). To test whether iaCMCs could be used to identify unintended Kᵥ11.1 blockers, the effect of exposing iaCMCs at day 6 of differentiation to 100 nM of the antihistamine astemizole, which was previously shown to efficiently block the Kᵥ11.1 channel, but no other cardiac ion channels (Delpón et al., 1999), was studied by optical voltage mapping. Astemizole significantly increased APD at 40 and 90% repolarization (APD₄₀ and APD₉₀, respectively) from 21.5±1.0 and 88.7±5.4 ms in vehicle-treated cultures to 30.2±2.1 and 110.9±4.2 ms in cultures containing 100 nM astemizole (N=6, P<0.01; FIG. 11, Panels B and C) without affecting CV (14.8±2.8 cm/s vs 14.5±1.1 cm/s in control cultures; N=6, P=0.57; FIG. 11, Panels D and E). These data demonstrate the presence of a detectable I_{Kr} in differentiating iaCMCs and show the utility of naCMCs cultures as in vitro model to investigate the AP-prolonging effects of Kᵥ11.1 blockers like astemizole.

Engraftment of iaCMCs into Viable Neonatal Rat Atrium

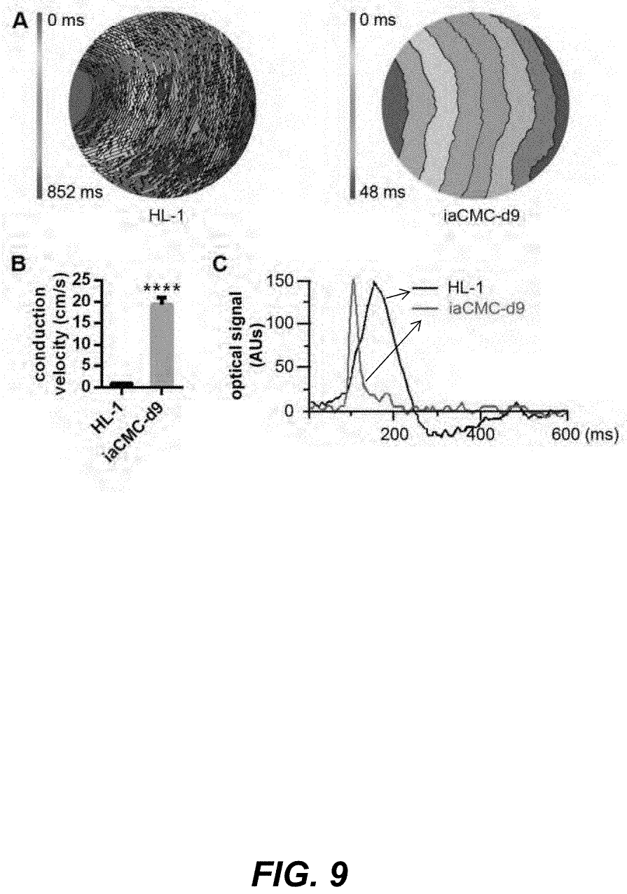

To investigate whether they were able to form a functional cardiac syncytium with naCMCs, iaCMCs were labeled with eGFP by lentiviral transduction. Next, 1:1 co-cultures of naCMCs treated at day 1 of culture with mitomycin C and eGFP-labeled iaCMCs were established and subsequently kept for 8 days in Dox-free culture medium. The co-cultures were compared with iaCMC monocultures at day 9 of differentiation and with 9-day-old, mitomycin C-treated naCMC cultures. Light microscopic analysis showed the eGFP-labeled iaCMCs were evenly distributed throughout the co-cultures and formed a tight monolayer with the surrounding naCMCs (FIG. 12, Panel A). Cx43 immunostaining of the co-cultures revealed the presence of gap junctions between iaCMCs and naCMCs (FIG. 12, Panel B). Optical voltage mapping showed uniform radial spreading of APs from the pacing electrode at roughly the same speed in all 3 culture types (FIG. 12, Panels C and D), suggesting proper electrical integration of differentiated iaCMCs in co-cultures with naCMCs.

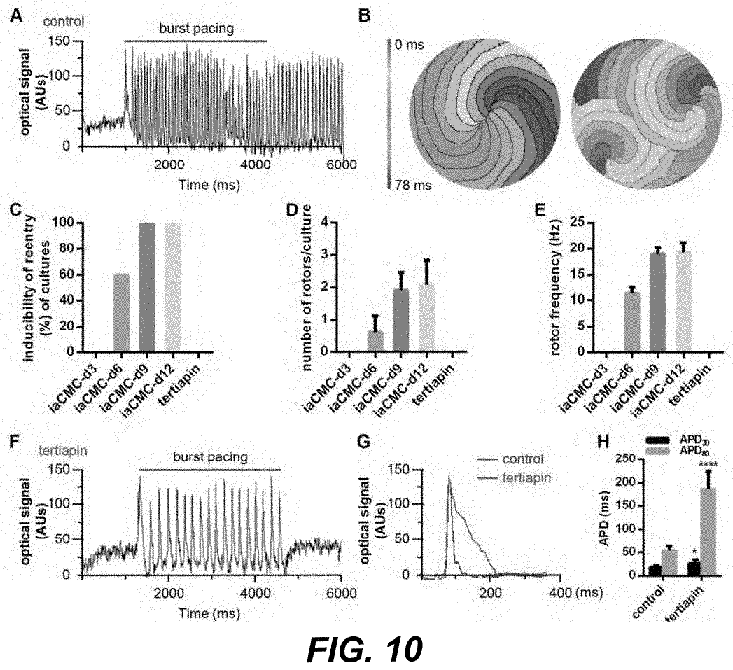

Based on this result, a transplantation model was developed to study the engraftment of iaCMCs in neonatal rat atrium (FIG. 13, Panel A). eGFP-labeled iaCMCs were cultured for 2 days in Dox-free medium to initiate cardiomyogenic differentiation and subsequently injected into the atrium. Following iaCMC injection, the atrium was excised from the heart and kept in culture for 6 days. The presence of spontaneous contractions at the end of the culture period demonstrated that the atrial tissue was still viable after 6 days of culture. Confocal laser scanning microscopy of the tissue after fixation at the end of the culture period revealed the presence of eGFP⁺ cells throughout the atrium (FIG. 13, Panel B). Immunostaining for α-actinin showed that many of the eGFP⁺ cells possessed highly organized sarcomeres (FIG. 13, Panel B), whose length (i.e., distance between 2 Z-lines) did not significantly differ from that of naCMCs (FIG. 13, Panel C). Collectively, these data provide evidence for the cardiomyogenic differentiation and structural integration of transplanted iaCMCs into atrial tissue.

Plasmid DNA Transfection of iaCMCs

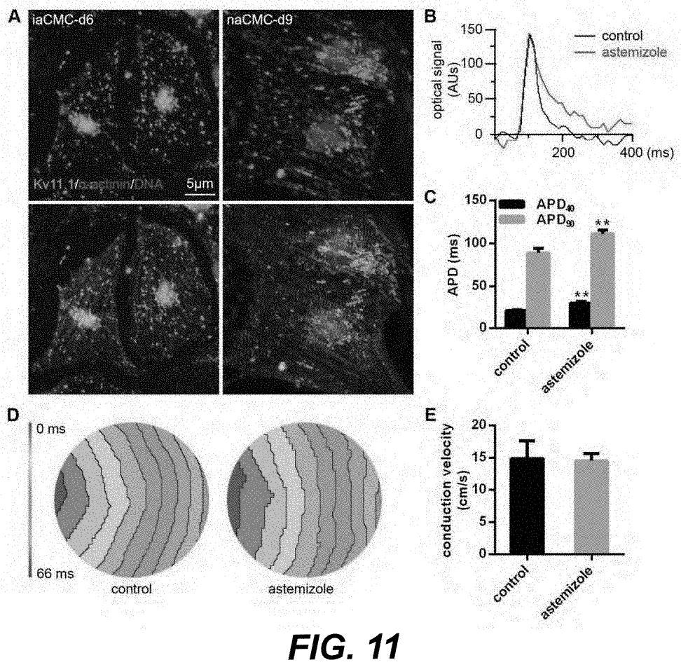

The practical applicability of iaCMCs would benefit greatly from them being well transfectable with plasmid DNA, especially since most viral gene transfer vectors (e.g., lentiviral and adeno-associated virus vectors) can accommodate only a limited amount of foreign genetic information. It was then tested whether iaCMCs would be amenable to plasmid DNA transfection with Lipofectamine 3000. Near-confluent cultures of proliferating iaCMCs were lipofected with the eGFP-encoding mammalian expression plasmid pU.CAG.eGFP in Dox-free medium and transgene expression was assessed by direct fluorescence microscopy and flow cytometry at 2 and 9 days after Dox removal (FIG. 14, Panel A and B). At 2 days post-transfection, approximately 50% of the iaCMCs were eGFP⁺, which percentage did not decrease following incubation of the cells in Dox-free medium for 7 more days. Importantly, transfected cells differentiated equally well into functional cardiomyocytes as their non-transduced counterparts judged from the results of α-actinin immunostaining (FIG. 14, Panel C) and optical voltage mapping (FIG. 14, Panel D) of the iaCMC cultures at 9 day after transfection.

### Summary and Conclusion

Until now, all attempts to generate lines of cardiomyocytes that can give rise to cells with electrical and contractile properties approaching those of the starting material have failed. Indeed, all cardiomyocyte lines reported to date display large structural and functional deficits compared to the primary cardiomyocytes from which they were derived (see e.g., Claycomb et al., 1998; Davidson et al., 2005; Zhang et al., 2009; Dias et al., 2014). In this study, it is described that the generation of monoclonal lines of conditionally immortalized neonatal rat atrial myocytes with preserved cardiomyogenic differentiation capacity. This was accomplished by transducing naCMCs with a lentiviral vector directing myocyte-selective and Dox-dependent expression of the SV40 LT antigen. Culturing of the resulting cells in medium with Dox caused their rapid expansion, while in Dox-free culture medium, the amplified cells spontaneously and synchronously differentiated into fully functional (i.e., excitable and contractile) naCMCs that were successfully used to develop a monolayer model of AF and for studying the APD-prolonging effects of drugs (astemizole) and toxins (tertiapin). To provide the iaCMCs with a unique and easy-to-remember identifier, the progeny of iaCMC clone #5 was dubbed the iAM-One cell line, which stands for the first line of reversibly immortalized neonatal rat atrial myocytes.

Of all cardiomyocyte lines generated to date, the murine atrial myocyte line HL-1 has the most differentiated phenotype and is therefore most widely used. Side-by-side comparison of the electrophysiological properties of confluent monolayers of differentiated iaCMCs and HL-1 cells by optical voltage mapping showed an average CV for the HL-1 cultures of 1 cm/s, which is about 20-fold lower than that of iaCMC cultures and in line with previous reports (Dias et al., 2014). Since Ma et al reported a resting membrane potential of −77.7±0.4 mV for HL-1 cells at an extracellular K⁺ concentration of 4 mM, the low CV in confluent HL-1 cell monolayers does not seem to result from a high percentage of cardiac fast sodium channels being in an inactivated state. The difference in CV between both cell types thus conceivably relates to the fact that confluent HL-1 monolayers are a blend of proliferating cells without cardiomyocyte functionality are electrically coupled to cells that phenotypically resemble embryonic atrial myocytes (Claycomb et al., 1998). Conversely, in the iaCMC cultures at 9 day of differentiation virtually all cells are in a similar advanced stage of differentiation forming a highly conductive cardiac syncytium (FIGS. 5A-5D and 7A-7K). Gap junctional coupling between undifferentiated and differentiated cells may also explain the apparent discrepancy between the broad optical voltage traces of HL-1 cells in monolayer cultures (FIG. 9, Panel C) versus the short APDs recorded for single HL-1 cells following whole-cell current clamping (Dias et al., 2014).

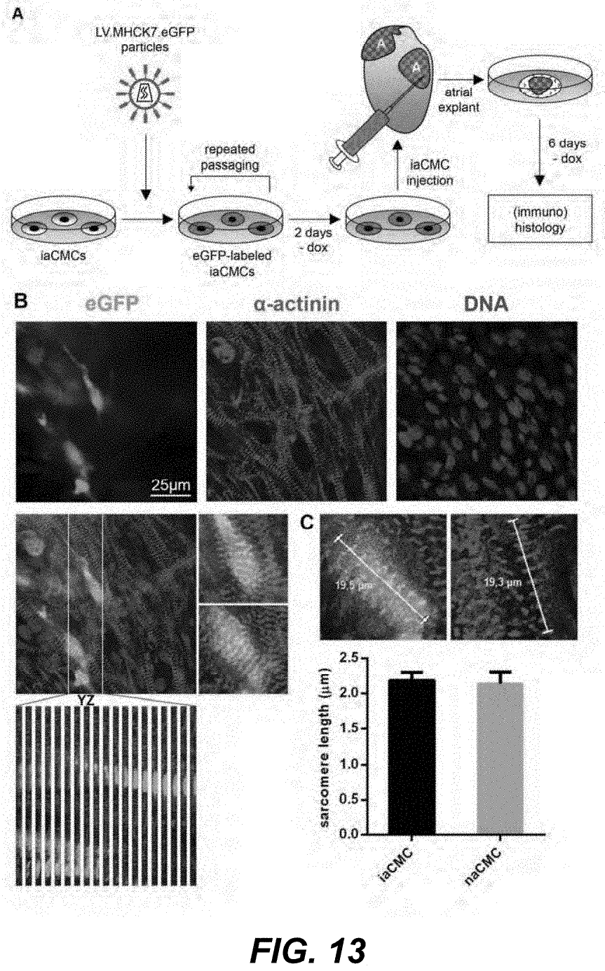

Long-term culture of iaCMCs did not significantly reduce their proliferation rate in the presence of Dox and the sarcomeric organization and contractile behavior of iaCMCs from different passages at day 9 of differentiation were indistinguishable (data not shown). Moreover, no progressive cell flattening, nuclear enlargement, development of senescence-associated heterochromatin foci or other signs of senescence were observed during the first 55 population doublings of the cells. Assessment of the electrophysiological properties of confluent monolayers of differentiated iaCMCs by optical voltage mapping after 25, 40 and 55 population doublings revealed no significant differences in APD or CV (FIG. 16). Also, differentiated iaCMCs resume cell division following addition of Dox to the culture medium and redifferentiate again into atrial myocytes following subsequent Dox removal (data not shown). iaCMCs therefore provide a virtually unlimited supply of cardiomyocytes for diverse applications including: (1) development of acquired disease models, (2) identification of new therapeutic targets, (3) screening of drugs and toxins, (4) testing of new treatment modalities (e.g., gene and cell therapy), (5) production of biopharmaceuticals in bioreactors or encapsulated cell/tissue grafts, (6) improvement of tissue engineering approaches and (7) fundamental research. The recent rapid progress in genome editing technologies will further expand the applicability of iaCMCs allowing them to be used as models for studying key aspects of inherited cardiomyopathies.

A highly attractive feature of the iaCMCs is that they spontaneously undergo cardiomyogenic differentiation in a synchronous and near quantitative manner in standard culture medium without Dox. Contrarily, generation of cardiomyocytes from embryonic or induced PSCs requires their properly timed treatment with various cocktails of growth factors and small molecules (Spater et al., 2014). This typically yields phenotypically heterogeneous populations of mostly immature cardiomyocytes contaminated with variable percentages of non-cardiomyocytes. Consequently, to obtain relatively pure populations of PSC-derived cardiomyocytes various different enrichment procedures have been developed. Production of more or less homogeneous populations of cardiomyocytes from PSCs thus is a laborious, time-consuming and costly endeavor as compared to the straightforward generation of atrial myocytes from iaCMCs. This may explain why there are very few reports on the use of PSC-CMCs to investigate cardiac arrhythmias in monolayer cultures by optical mapping (Herron, 2016), while such experiments are easily performed with differentiated iaCMC monolayers as evinced by the results presented in FIGS. 7A-7K through 12, 14, 16 and 17. Also, in contrast to PSC-CMCs, iAMs offer the possibility to study the molecular mechanisms involved in the transition of a differentiated cardiomyocyte that is no longer able to undergo cytokinesis into an actively dividing cell. This is particularly relevant given the growing interest in regeneration of mammalian hearts from within by stimulating cardiomyocytes surrounding the site(s) of cardiac injury to multiply themselves through cell division. Current findings regarding the molecular changes accompanying the reactivation of cardiomyocyte proliferation should be interpreted with caution due to the presence of other cell types (e.g., cardiac fibroblasts and inflammatory cells) in most test materials. iAMs do not suffer from this drawback making them a potentially highly useful model system to study the mechanisms underlying cell cycle reentry and progression of cardiomyocytes.

Although single-cell patch-clamp analysis can yield a wealth of information about the electrophysiological behavior of single cells and has been widely used to estimate the risk of drug-induced proarrhythmia, the technique does not directly assess proarrhythmic potential and is of limited use for studying arrhythmia mechanisms. Optical mapping, on the other hand, allows investigation of normal and disturbed cardiac electrical impulse propagation in a direct manner with high spatial and temporal resolution. As demonstrated by the experiments described in FIGS. 7A-7K through 12, 14, 16 and 17, confluent iaCMC cultures are well-suited for optical mapping studies. In combination with their large proliferation capacity, this opens perspectives for developing high-throughput analyses based on the iaCMC lines and/or future lines of other cardiomyocytes generated with the conditional immortalization method. Also in this case, the ability to genetically modify the iaCMCs will further increase their applicability.

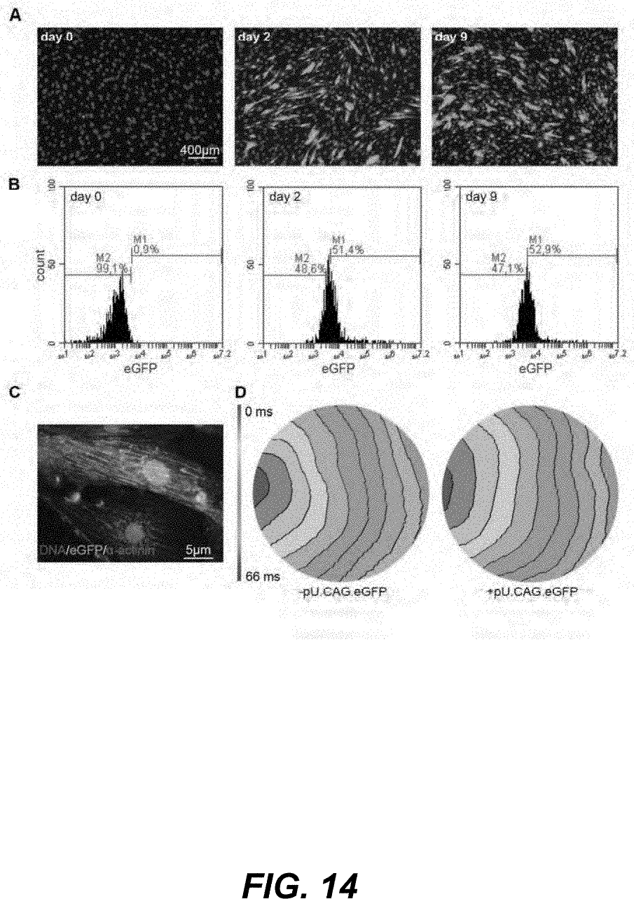

Following their differentiation in the absence of Dox, iaCMCs acquire properties of atrial rather than ventricular myocytes as evinced by the high-level expression in differentiated iaCMCs of “atrial” genes including Nppa, Myl7, Kcnj3 and Kcnj5 and the lack of expression of the “ventricular” Myh7 gene. Moreover, following 1-Hz pacing, differentiated iaCMCs display rapid contractions that strongly resemble those of naCMCs but are quite different from the slower contractions of nvCMCs (data not shown). Consistently, the average APD₃₀ and APD₈₀ of differentiated iaCMCs do not significantly differ from those of naCMCs (FIGS. 7J and 7K, respectively) but are much shorter than the average APD₃₀ and APD₈₀ of nvCMCs (FIG. 17). Collectively, these findings indicate that although LT expression results in the dedifferentiation and proliferation of naCMCs, the cells retain some kind of epigenetic memory causing them to spontaneously differentiate into atrial myocytes upon Dox removal.

RT-qPCR analysis of iaCMCs in different stages of differentiation (FIGS. 5A-5D and 6) shows a rapid increase in mRNA levels of cardiac transcription factors, ion channels, Ca²⁺ handling proteins and sarcomeric proteins shortly after Dox removal. At later stages of differentiation, these mRNA levels either stay fairly constant (as for the genes encoding cardiac transcription factors, sarcomeric and Ca²⁺ handling proteins) or decline again to the approximate mRNA levels of naCMCs (Scn5a, Cacna1c, Kcnj3, Kcnj11) and/or proliferating iaCMCs (Scn5a, Cacna1c, Kcnd3, Kcnh2, Kcnj3). These findings are consistent with the notion that upon Dox removal iaCMCs rapidly engage in a cardiomyogenic differentiation program with the concomitant upregulation of genes needed to generate a functional cardiomyocyte.

Comparison of the cell capacitance of iaCMCs at different days after Dox withdrawal with that of naCMCs at culture day 2-4 indicated that differentiated iaCMCs are considerably larger than naCMCs (FIG. 7E). This may relate to the different lengths of the culture periods and to the fact that the iaCMCs were cultured in the presence of 10% heat-inactivated FBS, whereas naCMCs were maintained in medium containing 5% heat-inactivating HS and therefore a much lower concentration of pro-hypertrophic growth factors. Interestingly, the difference in cell size between naCMCs and differentiated iaCMCs was only evident in very low-density cultures (data not shown).

In conclusion, by employing a monopartite lentiviral vector system allowing inducible expression of the SV40 LT antigen in cardiomyocytes, lines were generated of iaCMCs with (functional) properties vastly superior to those of all existing cardiomyocyte lines, none of which yields cells that even remotely approach the structural and functional maturity of Dox-deprived iaCMCs. The broad applicability of the iaCMCs was illustrated by their use for identifying drugs/toxins with cardiac ion channel-modulatory activity, as model of AF and for cardiac cell transplantation studies. Since iaCMCs undergo a highly coordinated and precisely timed cardiomyogenic differentiation process, they might also be ideally suited to study specific aspects of cardiac muscle cell formation and maturation. Finally, due to the fact that differentiation of iaCMCs into electrically and mechanically active cardiomyocytes occurs spontaneously without the need for complex treatment regimens with expensive (bio)chemicals, iaCMCs may provide an easy and cost-effective alternative for PSC-derived cardiomyocytes, especially when the conditional immortalization strategy can be expanded to other types of cardiomyocytes besides atrial myocytes and to human cardiac muscle cells.

### REFERENCES

Bingen B. O. et al. Cardiovasc. Res. 2014; 104:194-205.
Bingen B. O. et al. Circulation 2013; 128:2732-2744.
Bonger K. M. et al. Nat. Chem. Biol. 2011; 7:531-537.
Brandenburger M. et al. Cardiovasc. Res. 2012; 93:50-59.
Claycomb W. C. et al. Proc. Natl. Acad. Sci. USA 1998; 95:2979-2984.
Davidson M. M. et al. J. Mol. Cell. Cardiol. 2005; 39:133-147.
Delpón E. et al. Drug Saf. 1999; 21 Suppl 1:11-18.
Dias P. et al. PLoS One 2014; 9:e90266.
Dinsart C. et al. Mol. Cell. Biol. 1984; 4:324-328.
Feola I. et al. Methods Mol. Biol. 2016; 1408:319-331.
Goldman B. I. et al. In Vitro Cell. Dev. Biol. Anim. 2006; 42:324-331.
Herron T. J. Cell Calcium 2016; 59:84-90.
Holland A. J. et al. Proc. Natl. Acad. Sci. USA 2012; 109:E3350-E3357.
Jahn L. et al. J. Cell. Sci. 1996; 109:397-407.
Kannankeril P. et al. Pharmacol. Rev. 2010; 62:760-781.
Kubalak S. W. et al. J. Biol. Chem. 1994; 269:16961-16970.
Loeber G. et al. J. Virol. 1989; 63:4426-4430.
Lufino M. M. et al. Mol. Ther. 2008; 16:1525-1538.
Ma L., Zhang X., Chen H. Sci. Signal. 2011; 4: ra37 Majumder R. et al. Sci. Rep. 2016 Apr. 13; 6:24334. Epub ahead of print.
Navarro R. et al. ACS Chem. Biol. 2016; 11:2101-2104.
Niwa H. et al. Gene 1991; 108:193-199.
Ramkisoensing A. A. et al. Stem Cells 2012; 30:2830-2834.
Rybkin I I et al. J. Biol. Chem. 2003; 278:15927-15934.
Salva M. Z. et al. Mol. Ther. 2007; 15:320-329.
Schambach A. et al. Gene Ther. 2006; 13:641-645.
Schott J. W. et al. Mol. Ther. 2016; 24:1513-1527.
Spater D. et al. Development 2014; 141:4418-4431.
Steinhelper M. E. et al. Am. J. Physiol. 1990; 259:H1826-H1834.
Szulc J. et al. Nat. Methods 2006; 3:109-116. van Tuyn J. et al. FASEB J. 2007; 21:3369-3379.
Weber W., Fussenegger M. J. Gene Med. 2006; 8:535-56.
White S. M. et al. Am. J. Physiol. Heart. Circ. Physiol. 2004; 286:H823-H829.
Woodard L. E., Calos M. P. (2015). Chapter 26, in Gene and Cell Therapy Therapeutic Mechanisms and Strategies, Fourth Edition, ed. Templeton N S, CRC Press, pp. 675-700. et al.
Yu Z. et al. Circ. Arrhythm. Electrophysiol. 2016 April; 9:e003439; Epub ahead of print.
Zhang Y. et al. Hum. Gene Ther. 2009; 20:1687-1696.
Zhang L. et al. Sci. China Life Sci. 2013; 56:788-796.

1. A genetically modified eukaryotic cell provided with a vector, the vector comprising:
a promoter;
at least one immortalization gene operably linked to the promoter;
a gene coding for an inducible regulator operably linked to the promoter; and
a response element for the inducible regulator.

2. The cell according to claim 1 wherein the promoter is a cell type- or tissue-specific promoter and/or a promoter that is active in a differentiated form of the cell.

3. The cell according to claim 1, wherein the cell is a primary cell or progeny thereof.

4. The cell according to of claim 1, wherein the at least one immortalization gene comprises a simian virus 40 large T antigen (LT) gene or a variant thereof.

5. The cell of claim 1, wherein the inducible regulator is a tetracycline (Tc)-responsive transcriptional repressor, and the response element is a Tc response element (TRE).

6. The cell of claim 1, wherein the cell is a cardiac cell and the promoter is a differentiated cardiac cell-specific promoter.

7. The cell of claim 1 wherein said cell is a cardiac cell.

8. The cell claim 1, wherein the promoter is the chimeric striated muscle-specific MHCK7 promoter.

9. A cell line comprising a plurality of cells according to claim 1.

10. A kit of parts comprising:
the cell of claim 1 or cell line thereof, and
an agent capable of activating or repressing the in promoter.

11. The kit of parts according to claim 10 wherein the promoter is a Tc-inducible promoter and the agent is a Tc class antibiotic.

12. A method for generating one or more conditionally immortalized cells, the method comprising:
introducing at least one vector comprising:
a promoter
at least one immortalization gene operably linked to the promoter,
a gene coding for an inducible regulator operably linked to the promoter, and
a response element for the inducible regulator into one or more eukaryotic cells resulting in one or more genetically modified eukaryotic cells.

13. The method according to claim 12, further comprising culturing the one or more conditionally immortalized cells under conditions whereby transcription of the one or more immortalization genes is induced.

14. The method according to claim 13, comprising culturing primary cells provided with the one or more vectors in the presence of an agent that induces release of an inducible repressor from the response element thereby activating the promoter and allowing transcription of the at least one immortalization gene.

15. The method according to claim 12, further comprising:
culturing the one or more conditionally immortalized cells under conditions whereby transcription of the one or more immortalization genes is inhibited.

16. The cell of claim 4, wherein the LT gene encodes a temperature-sensitive variant.

17. The cell of claim 16, wherein the LT gene encodes temperature-sensitive mutant tsA58.

18. The cell of claim 7, wherein the cardiac cell is a cardiomyocyte, cardiac fibroblast, or pericardial mesothelial cell.
